(19) United States
(12) Patent Application Publication
(10) Pub. No.: US 2023/0227545 A1
(43) Pub. Date: Jul. 20, 2023

(54) MATERIALS AND METHODS OF IL-1BETA BINDING PROTEINS

(71) Applicant: JOHNSON & JOHNSON ENTERPRISE INNOVATION INC., New Brunswick, NJ (US)

(72) Inventors: Gabriel PASCUAL, San Diego, CA (US); Sagit HINDI, Wayne, PA (US); Suresh Kumar SWAMINATHAN, Lansdale, PA (US)

(73) Assignee: JOHNSON & JOHNSON ENTERPRISE INNOVATION INC., New Brunswick, NJ (US)

(21) Appl. No.: 18/151,305

(22) Filed: Jan. 6, 2023

Related U.S. Application Data
(60) Provisional application No. 63/297,436, filed on Jan. 7, 2022.

(51) Int. Cl. C07K 16/24

(52) U.S. Cl. CPC C07K 16/245; C07K 2317/31; C07K 2317/76; C07K 2317/33; C07K 2317/92

(57) ABSTRACT

Provided herein, in certain aspects, are antibodies that bind to IL-1β and compositions comprising the antibodies. Methods of making and using the antibodies are also provided.

### CROSS-REFERENCE TO RELATED APPLICATIONS

This application claims benefit of U.S. Provisional Application No. 63/297,436, filed Jan. 7, 2022, the disclosure of which is incorporated by reference herein in its entirety.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14620-563-999_SEQ_LISTING.xml”, was created on Dec. 1, 2022, and is 123,349 bytes in size.

### 1. FIELD

This disclosure relates to anti-IL-1β antibodies, nucleic acids and expression vectors encoding the antibodies, recombinant cells containing the vectors, and compositions comprising the antibodies. Methods of making the antibodies and methods of using the antibodies to treat diseases including cancer are also provided.

### 2. BACKGROUND

IL-1β is a pleiotropic cytokine with numerous roles in both physiological and pathological states. In cancer, IL-1β facilitates a tumor-supportive microenvironment through a variety of mechanisms. For example, IL-1β has been suggested to promote the production of mutagenic reactive oxygen species that can lead to tumor development. (Taniguchi K et al, Nat Rev Immunol. 2018; 18(5):309-324.) In addition, the IL-1 pathway promotes the expression of vascular endothelial growth factor (VEGF) and fibroblast growth factor (FGF), two key proangiogenic factors that lead to neo-formation of capillaries, a hallmark of tumor progression and essential for tumor invasiveness and metastasis. (Voronov E et al, Proc Natl Acad Sci USA. 2003; 100(5):2645-2650; Voronov E et al, Front Physiol. 2014; 5:114.) IL-1β also promotes epithelial to mesenchymal transition (EMT) in vitro, which is a critical step in the early phase of the metastatic cascade. (Li R et al, Sci Rep. 2020; 10(1):377.) Within the TME, IL-1β can recruit and reprogram multiple cell types; for example, IL-1β has been shown to promote macrophage and neutrophil infiltration, mobilize immunosuppressive myeloid populations (e.g., MDSCs, TAMs, and TANs), and dampen anti-tumor T cell infiltration and activation (Bunt S K et al, J Immunol. 2006; 176(1):284-290.)

Furthermore, clinical data provided evidence for an important role of the IL-1 pathway in cancer. (Ridker P M et al, Lancet. 2017; 390(10105):1833-1842.) Thus, there is a need in the art for a high-affinity and potent anti-IL-1β antibody molecules capable of neutralizing the IL-1β signaling pathway for cancer treatment.

### 3. SUMMARY

In one aspect, provided herein is an antibody that binds IL-1β comprising:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 8;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 9; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 10; or
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 11; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 12.

In some embodiments, (i) the VH CDR1, VH CDR2, VH CDR3, VL CDR1, VL CDR2, and VL CDR3 amino acid sequences are according to the Kabat numbering system; (ii) the VH CDR1, VH CDR2, VH CDR3, VL CDR1, VL CDR2, and VL CDR3 amino acid sequences are according to the Chothia numbering system; (iii) the VH CDR1, VH CDR2, VH CDR3, VL CDR1, VL CDR2, and VL CDR3 amino acid sequences are according to the AbM numbering system; (iv) the VH CDR1, VH CDR2, VH CDR3, VL CDR1, VL CDR2, and VL CDR3 amino acid sequences are according to the Contact numbering system; and/or (v) the VH CDR1, VH CDR2, VH CDR3, VL CDR1, VL CDR2, and VL CDR3 amino acid sequences are according to the IMGT numbering system.

In another aspect, provided herein is an antibody that binds IL-1β comprising:
(1) (i) a VH comprising a VH CDR1 having an amino acid sequence selected from SEQ ID NO: 13, SEQ ID NO: 31, SEQ ID NO: 49, SEQ ID NO: 67, and SEQ ID NO: 85; a VH CDR2 having an amino acid sequence selected from SEQ ID NO: 14, SEQ ID NO: 32, SEQ ID NO: 50, SEQ ID NO: 68, and SEQ ID NO: 86; a VH CDR3 having an amino acid sequence selected from SEQ ID NO: 15, SEQ ID NO: 33, SEQ ID NO: 51, SEQ ID NO: 69, and SEQ ID NO: 87; and
   (ii) a VL comprising a VL CDR1 having an amino acid sequence selected from SEQ ID NO: 16, SEQ ID NO: 34, SEQ ID NO: 52, SEQ ID NO: 70, and SEQ ID NO: 88; a VL CDR2 having an amino acid sequence selected from SEQ ID NO: 17, SEQ ID NO: 35, SEQ ID NO: 53, SEQ ID NO: 71, and SEQ ID NO: 89; a VL CDR3 having an amino acid sequence selected from SEQ ID NO: 18, SEQ ID NO: 36, SEQ ID NO: 54, SEQ ID NO: 72, and SEQ ID NO: 90;
(2) (i) a VH comprising a VH CDR1 having an amino acid sequence selected from SEQ ID NO: 19, SEQ ID NO: 37, SEQ ID NO: 55, SEQ ID NO: 73, and SEQ ID NO: 91; a VH CDR2 having an amino acid sequence selected from SEQ ID NO: 20, SEQ ID NO: 38, SEQ ID NO: 56, SEQ ID NO: 74, and SEQ ID NO: 92; a VH CDR3 having an amino acid sequence selected from SEQ ID NO: 21, SEQ ID NO: 39, SEQ ID NO: 57, SEQ ID NO: 75, and SEQ ID NO: 93; and
   (ii) a VL comprising a VL CDR1 having an amino acid sequence selected from SEQ ID NO: 22, SEQ ID NO: 40, SEQ ID NO: 58, SEQ ID NO: 76, and SEQ ID NO: 94; a VL CDR2 having an amino acid sequence selected from SEQ ID NO: 23, SEQ ID NO: 41, SEQ ID NO: 59, SEQ ID NO: 77, and SEQ ID NO: 95; a VL CDR3 having an amino acid sequence selected from SEQ ID NO: 24, SEQ ID NO: 42, SEQ ID NO: 60, SEQ ID NO: 78, and SEQ ID NO: 96; or
(3) (i) a VH comprising a VH CDR1 having an amino acid sequence selected from SEQ ID NO: 25, SEQ ID NO: 43, SEQ ID NO: 61, SEQ ID NO: 79, and SEQ ID NO: 97; a VH CDR2 having an amino acid sequence selected from SEQ ID NO: 26, SEQ ID NO: 44, SEQ ID NO: 62, SEQ ID NO: 80, and SEQ ID NO: 98; a VH CDR3 having an amino acid sequence selected from SEQ ID NO: 27, SEQ ID NO: 45, SEQ ID NO: 63, SEQ ID NO: 81, and SEQ ID NO: 99; and
   (ii) a VL comprising a VL CDR1 having an amino acid sequence selected from SEQ ID NO: 28, SEQ ID NO: 46, SEQ ID NO: 64, SEQ ID NO: 82, and SEQ ID NO: 100; a VL CDR2 having an amino acid sequence selected from SEQ ID NO: 29, SEQ ID NO: 47, SEQ ID NO: 65, SEQ ID NO: 83, and SEQ ID NO: 101; a VL CDR3 having an amino acid sequence selected from SEQ ID NO: 30, SEQ ID NO: 48, SEQ ID NO: 66, SEQ ID NO: 84, and SEQ ID NO: 102.

In another aspect, provided herein is an antibody that binds IL-1β comprising:
(1) (i) a VH comprising a VH CDR1 having an amino acid sequence of SEQ ID NO: 13; a VH CDR2 having an amino acid sequence of SEQ ID NO: 14; a VH CDR3 having an amino acid sequence of SEQ ID NO: 15; and
   (ii) a VL comprising a VL CDR1 having an amino acid sequence of SEQ ID NO: 16; a VL CDR2 having an amino acid sequence selected of SEQ ID NO: 17; a VL CDR3 having an amino acid sequence of SEQ ID NO: 18;
(2) (i) a VH comprising a VH CDR1 having an amino acid sequence of SEQ ID NO: 19; a VH CDR2 having an amino acid sequence of SEQ ID NO: 20; a VH CDR3 having an amino acid sequence of SEQ ID NO: 21; and
   (ii) a VL comprising a VL CDR1 having an amino acid sequence of SEQ ID NO: 22; a VL CDR2 having an amino acid sequence selected of SEQ ID NO: 23; a VL CDR3 having an amino acid sequence of SEQ ID NO: 24;
(3) (i) a VH comprising a VH CDR1 having an amino acid sequence of SEQ ID NO: 25; a VH CDR2 having an amino acid sequence of SEQ ID NO: 26; a VH CDR3 having an amino acid sequence of SEQ ID NO: 27; and
   (ii) a VL comprising a VL CDR1 having an amino acid sequence of SEQ ID NO: 28; a VL CDR2 having an amino acid sequence selected of SEQ ID NO: 29; a VL CDR3 having an amino acid sequence of SEQ ID NO: 30;
(4) (i) a VH comprising a VH CDR1 having an amino acid sequence of SEQ ID NO: 31; a VH CDR2 having an amino acid sequence of SEQ ID NO: 32; a VH CDR3 having an amino acid sequence of SEQ ID NO: 33; and
   (ii) a VL comprising a VL CDR1 having an amino acid sequence of SEQ ID NO: 34; a VL CDR2 having an amino acid sequence selected of SEQ ID NO: 35; a VL CDR3 having an amino acid sequence of SEQ ID NO: 36;
(5) (i) a VH comprising a VH CDR1 having an amino acid sequence of SEQ ID NO: 37; a VH CDR2 having an amino acid sequence of SEQ ID NO: 38; a VH CDR3 having an amino acid sequence of SEQ ID NO: 39; and
   (ii) a VL comprising a VL CDR1 having an amino acid sequence of SEQ ID NO: 40; a VL CDR2 having an amino acid sequence selected of SEQ ID NO: 41; a VL CDR3 having an amino acid sequence of SEQ ID NO: 42;
(6) (i) a VH comprising a VH CDR1 having an amino acid sequence of SEQ ID NO: 43; a VH CDR2 having an amino acid sequence of SEQ ID NO: 44; a VH CDR3 having an amino acid sequence of SEQ ID NO: 45; and
   (ii) a VL comprising a VL CDR1 having an amino acid sequence of SEQ ID NO: 46; a VL CDR2 having an amino acid sequence selected of SEQ ID NO: 47; a VL CDR3 having an amino acid sequence of SEQ ID NO: 48;
(7) (i) a VH comprising a VH CDR1 having an amino acid sequence of SEQ ID NO: 49; a VH CDR2 having an amino acid sequence of SEQ ID NO: 50; a VH CDR3 having an amino acid sequence of SEQ ID NO: 51; and
   (ii) a VL comprising a VL CDR1 having an amino acid sequence of SEQ ID NO: 52; a VL CDR2 having an amino acid sequence selected of SEQ ID NO: 53; a VL CDR3 having an amino acid sequence of SEQ ID NO: 54;
(8) (i) a VH comprising a VH CDR1 having an amino acid sequence of SEQ ID NO: 55; a VH CDR2 having an amino acid sequence of SEQ ID NO: 56; a VH CDR3 having an amino acid sequence of SEQ ID NO: 57; and
   (ii) a VL comprising a VL CDR1 having an amino acid sequence of SEQ ID NO: 58; a VL CDR2 having an amino acid sequence selected of SEQ ID NO: 59; a VL CDR3 having an amino acid sequence of SEQ ID NO: 60;
(9) (i) a VH comprising a VH CDR1 having an amino acid sequence of SEQ ID NO: 61; a VH CDR2 having an amino acid sequence of SEQ ID NO: 62; a VH CDR3 having an amino acid sequence of SEQ ID NO: 63; and
   (ii) a VL comprising a VL CDR1 having an amino acid sequence of SEQ ID NO: 64; a VL CDR2 having an amino acid sequence selected of SEQ ID NO: 65; a VL CDR3 having an amino acid sequence of SEQ ID NO: 66;
(10) (i) a VH comprising a VH CDR1 having an amino acid sequence of SEQ ID NO: 67; a VH CDR2 having an amino acid sequence of SEQ ID NO: 68; a VH CDR3 having an amino acid sequence of SEQ ID NO: 69; and
   (ii) a VL comprising a VL CDR1 having an amino acid sequence of SEQ ID NO: 70; a VL CDR2 having an amino acid sequence selected of SEQ ID NO: 71; a VL CDR3 having an amino acid sequence of SEQ ID NO: 72;
(11) (i) a VH comprising a VH CDR1 having an amino acid sequence of SEQ ID NO: 73; a VH CDR2 having an amino acid sequence of SEQ ID NO: 74; a VH CDR3 having an amino acid sequence of SEQ ID NO: 75; and
   (ii) a VL comprising a VL CDR1 having an amino acid sequence of SEQ ID NO: 76; a VL CDR2 having an amino acid sequence selected of SEQ ID NO: 77; a VL CDR3 having an amino acid sequence of SEQ ID NO: 78;
(12) (i) a VH comprising a VH CDR1 having an amino acid sequence of SEQ ID NO: 79; a VH CDR2 having an amino acid sequence of SEQ ID NO: 80; a VH CDR3 having an amino acid sequence of SEQ ID NO: 81; and
   (ii) a VL comprising a VL CDR1 having an amino acid sequence of SEQ ID NO: 82; a VL CDR2 having an amino acid sequence selected of SEQ ID NO: 83; a VL CDR3 having an amino acid sequence of SEQ ID NO: 84;
(13) (i) a VH comprising a VH CDR1 having an amino acid sequence of SEQ ID NO: 85; a VH CDR2 having an amino acid sequence of SEQ ID NO: 86; a VH CDR3 having an amino acid sequence of SEQ ID NO: 87; and
   (ii) a VL comprising a VL CDR1 having an amino acid sequence of SEQ ID NO: 88; a VL CDR2 having an amino acid sequence selected of SEQ ID NO: 89; a VL CDR3 having an amino acid sequence of SEQ ID NO: 90;
(14) (i) a VH comprising a VH CDR1 having an amino acid sequence of SEQ ID NO: 91; a VH CDR2 having an amino acid sequence of SEQ ID NO: 92; a VH CDR3 having an amino acid sequence of SEQ ID NO: 93; and
   (ii) a VL comprising a VL CDR1 having an amino acid sequence of SEQ ID NO: 94; a VL CDR2 having an amino acid sequence selected of SEQ ID NO: 95; a VL CDR3 having an amino acid sequence of SEQ ID NO: 96; or
(15) (i) a VH comprising a VH CDR1 having an amino acid sequence of SEQ ID NO: 97; a VH CDR2 having an amino acid sequence of SEQ ID NO: 98; a VH CDR3 having an amino acid sequence of SEQ ID NO: 99; and
   (ii) a VL comprising a VL CDR1 having an amino acid sequence of SEQ ID NO: 100; a VL CDR2 having an amino acid sequence selected of SEQ ID NO: 101; a VL CDR3 having an amino acid sequence of SEQ ID NO: 102.

In some embodiments, the antibody provided herein further comprises one or more framework regions as set forth in SEQ ID NO: 7, SEQ ID NO: 8, SEQ ID NO: 9, SEQ ID NO: 10, SEQ ID NO: 11, and/or SEQ ID NO: 12.

In some embodiments, (i) the antibody comprises a VH having an amino acid sequence of SEQ ID NO: 7, and a VL having an amino acid sequence of SEQ ID NO: 8; (ii) the antibody comprises a VH having an amino acid sequence of SEQ ID NO: 9, and a VL having an amino acid sequence of SEQ ID NO: 10; or (iii) the antibody comprises a VH having an amino acid sequence of SEQ ID NO: 11, and a VL having an amino acid sequence of SEQ ID NO: 12.

In some embodiments, the antibody provided herein is a humanized antibody.

In some embodiments, the antibody provided herein is an IgG antibody. In some embodiments, the IgG antibody is an IgG1, IgG2, IgG3, or IgG4 antibody.

In some embodiments, the antibody provided herein comprises a kappa light chain.

In some embodiments, the antibody provided herein comprises a lambda light chain.

In some embodiments, the antibody provided herein comprises a mutant Fc region. In some embodiments, the mutant Fc region comprises M252Y/S254T/T256E (YTE) mutations.

In some embodiments, the antibody provided herein is a monoclonal antibody.

In some embodiments, the antibody provided herein binds an IL-1β antigen.

In some embodiments, the antibody provided herein binds an IL-1β epitope.

In some embodiments, the antibody provided herein specifically binds to IL-1β.

In some embodiments, the VH CDR1, VH CDR2, VH CDR3, VL CDR1, VL CDR2 and VL CDR3 form a binding site for an antigen of the IL-1β.

In some embodiments, the VH CDR1, VH CDR2, VH CDR3, VL CDR1, VL CDR2 and VL CDR3 form a binding site for an epitope of the IL-1β.

In some embodiments, the antibody provided herein is multispecific. In some embodiments, the antibody provided herein is capable of binding at least two antigens. In some embodiments, the antibody provided herein is capable of binding at least three antigens. In some embodiments, the antibody provided herein is capable of binding at least four antigens. In some embodiments, the antibody provided herein is capable of binding at least five antigens.

In another aspect, provided herein is a binding molecule comprising the antibody provided herein. In some embodiments, the antibody is genetically fused or chemically conjugated to an agent.

In another aspect, provided herein is a nucleic acid encoding the antibody provided herein.

In another aspect, provided herein is a vector comprising the nucleic acid provided herein.

In another aspect, provided herein is a host cell comprising the vector provided herein.

In another aspect, provided herein is a kit comprising the vector provided herein and packaging for the same.

In another aspect, provided herein is a kit comprising the antibody provided herein and packaging for the same.

In another aspect, provided herein is a pharmaceutical composition comprising the antibody provided herein, and one or more pharmaceutically acceptable excipients.

In another aspect, provided herein is a method of producing the pharmaceutical composition provided herein, comprising combining the antibody with one or more pharmaceutically acceptable excipients to obtain the pharmaceutical composition.

In another aspect, provided herein is a method of inhibiting IL-1β or IL-1β mediated signaling in a cell, comprising the contacting the cell with the antibody provided herein.

In another aspect, provided herein is a method of inhibiting IL-1β induced production of IL-6, ENA-78 (CXCL5) and/or G-CSF in a cell, comprising the contacting the cell with the antibody provided herein.

In another aspect, provided herein is a method of decreasing the production of IL-6, ENA-78 (CXCL5) and/or G-CSF in a cell, comprising the contacting the cell with the antibody provided herein.

In another aspect, provided herein is a method inhibiting growth or proliferation of IL-1β expressing cells, comprising contacting the cells with the antibody provided herein.

In some embodiments, the cell or the cells are in a subject having a disease or disorder.

In another aspect, provided herein is a method of inhibiting IL-1β in a subject, comprising administering to the subject the antibody provided herein.

In another aspect, provided herein is a method for treating a disease or disorder in a subject, comprising administering to the subject the antibody provided herein.

In some embodiments, the disease or disorder is an IL-1β associated disease or disorder. In some embodiments, the IL-1β associated disease or disorder is an inflammatory disease or disorder. In some embodiments, the IL-1β associated disease or disorder is cancer. In some embodiments, the cancer is lung cancer. In some embodiments, the lung cancer is non-small cell lung cancer. In some embodiments, the non-small cell lung cancer has reached stage 0, stage 1, stage 2, stage 3, or stage 4. In some embodiments, the cancer is kidney cancer. In some embodiments, the kidney cancer is renal cell cancer. In some embodiments, the renal cell cancer has reached stage 1, stage 2, or stage 3.

### 4. BRIEF DESCRIPTION OF THE FIGURES

FIG. 1. The epitope mapping of select antibodies on IL-1β using the hydrogen-deuterium exchange-based LC-MS. Top, the sequence shown is the fragment of SEQ ID NO: 109, residues 117-269 corresponding to the sequence of the mature IL-1β protein (SEQ ID NO:110). Double underline indicates strong epitope (ΔΔG upon binding ≤−2 kcal/mol) and single underline indicates weak epitope (−2<ΔΔG upon binding ≤−1 kcal/mol). Bottom, the epitopes overlaid on X-ray crystal structure of IL-1β (PDB ID 1I1B). Black indicates strong epitope and gray indicates weak epitope.

FIG. 2: Potency of 05H21A, 08F17A, and 15N14A (all with YTE mutation in the Fc region) in an NF-kB/AP-1 reporter system. Neutralizing activity of lead anti-IL-1β antibody panel was assessed in HEK-Blue reporter cells. Dose-response curves and IC50 values of lead anti-IL-1β mAb panel are shown.

FIG. 3: The inhibitory activity of anti-IL-1β lead antibody panel was assessed in MRCS human lung fibroblast cells. Figure shows dose-response curves of lead anti-IL-1β mAb panel and corresponding IC50 determinations.

FIGS. 4A and 4B: Potency of 05H21A, 08F17A, and 15N14A in human lung fibroblasts. Neutralizing activity of lead anti-IL-1β antibody panel was assessed in normal human lung fibroblasts (NHLF donors 34325 and 35234). Figures show dose-response curves from donor 34325 based on IL-6 (FIG. 4A) and CXCL5 (FIG. 4B) release measurements with corresponding IC50 determinations reported.

FIGS. 5A and 5B: Potency of 05H21A, 08F17A, and 15N14A in human donor PBMC samples. Neutralizing activity of lead anti-IL-1β antibody panel was assessed in one healthy human donor PBMCs (donor TS235). (FIG. 5A) and (FIG. 5B) Dose-response curves measuring IL-6 release of lead panel and calculated IC50 values.

FIG. 6: Potency of 05H21A, 08F17A, and 15N14A in human blood assay. Neutralizing activity of lead anti-IL-1β antibody panel was assessed in human whole blood samples (donors tested: CC00448, M3767, M5988, M7286, and M7370). Plotted are IC50 values in nM based on IL-6, CXCL-5 and G-CSF release measurements by MSD.

FIGS. 7A and 7B: Potency of 05H21A, 08F17A, and 15N14A in cynomolgus macaque fibroblast samples. Neutralizing activity of lead anti-IL-1β antibody panel was assessed in primary cynomolgus dermal and lung fibroblasts (CDF and CLF, respectively). (FIG. 7A) and (FIG. 7B) Dose-response curves measuring IL-6 release of lead panel in CDF and CLF, respectively. Shown are calculated IC50 values for anti-IL-1β antibody panel.

### 5. DETAILED DESCRIPTION

The present disclosure is based in part on the novel antibodies that bind to IL-1β and superior properties thereof.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, antibodies including from Camelidae species (e.g., llama or alpaca) or their humanized variants, intrabodies, anti-idiotypic (anti-Id) antibodies, and functional fragments (e.g., antigen binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (MA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or MA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise an antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Bruggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MEW) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105--L117 |
| | | | L89--L97 | | |
| CDR H1 | H31--H35B | H26--H35B | H26-- | H30--H35B | H27--H38 |
| | (Kabat | | H32 . . . 34 | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein for a particular epitope of an antigen. Natural antibodies, for example, are monospecific. The term “multispecific” as used herein denotes that an antigen binding protein has two or more antigen-binding sites of which at least two bind different antigens. “Bispecific” as used herein denotes that an antigen binding protein has two different antigen-binding specificities. The term “monospecific” antibody as used herein denotes an antigen binding protein that has one or more binding sites each of which bind the same antigen.

The term “valent” as used herein denotes the presence of a specified number of binding sites in an antigen binding protein. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding an antibody described herein is a nucleic acid molecule that is identified and separated from at least one contaminant nucleic acid molecule with which it is ordinarily associated in the environment in which it was produced.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants; low molecular weight (e.g., fewer than about 10 amino acid residues) polypeptide; proteins; hydrophilic polymers; amino acids; monosaccharides, disaccharides, and other carbohydrates; chelating agents; sugar alcohols; salt-forming counterions; and/or nonionic surfactants. Non-limiting examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin. Water is an exemplary excipient when a composition (e.g., a pharmaceutical composition) is administered intravenously. Saline solutions and aqueous dextrose and glycerol solutions can also be employed as liquid excipients, particularly for injectable solutions. The composition, if desired, can also contain minor amounts of wetting or emulsifying agents, or pH buffering agents. Compositions can take the form of solutions, suspensions, emulsions, tablets, pills, capsules, powders, and the like.

Compositions, including pharmaceutical compositions, may contain a binding molecule (e.g., an antibody as described herein), for example, in isolated or purified form, together with a suitable amount of excipients.

The term “effective amount” or “therapeutically effective amount” as used herein refers to the amount of an antibody or a therapeutic molecule comprising an agent and the antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

The terms “intercept,” “intercepting,” and “interception” refer to administering a therapy early in a disease process (e.g., pre-symptomatic and/or premalignant disease) to prevent, inhibit or delay disease progression (e.g., to prevent, inhibit or delay progression to late-stage cancer, such as malignant cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

“IL-1β associated disease or disorder” as used herein refers to a disease or disorder that comprises a cell or tissue in which IL-1β is expressed or overexpressed. In some embodiments, IL-1β associated disease or disorder comprises a cell on which IL-1β is abnormally expressed. In other embodiments, IL-1β associated disease or disorder comprises a cell in or on which IL-1β is deficient in at least one of its activities.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. IL-1β Binding Molecules

5.2.1. Antibodies that Bind to IL-1β

In one aspect, provided herein are antibodies capable of binding to IL-1β. IL-1β is a pleiotropic cytokine with numerous roles in both physiological and pathological states. In cancer, IL-1β facilitates a tumor-supportive microenvironment through a variety of mechanisms. In addition, the IL-1 pathway promotes the expression of vascular endothelial growth factor (VEGF) and fibroblast growth factor (FGF), two key proangiogenic factors that lead to neo-formation of capillaries, a hallmark of tumor progression and essential for tumor invasiveness and metastasis. IL-1β also promotes epithelial to mesenchymal transition (EMT) in vitro, which is a critical step in the early phase of the metastatic cascade. IL-1β can recruit and reprogram multiple cell types; for example, IL-1β has been shown to promote macrophage and neutrophil infiltration, mobilize immunosuppressive myeloid populations (e.g., MDSCs), enhance neutrophil infiltration, and dampen T cell infiltration and activation. Nucleic acid and amino acid sequences of IL-1β are known (see GCID: GC02M112829, HGNC: 5992, NCBI Entrez Gene: 3553, Ensembl: ENSG00000125538, OMIM®: 147720, and UniProtKB/Swiss-Prot: P01584). In some embodiments, the antibodies provided herein bind to human IL-1β. In some embodiments, the anti-IL-1β antibody provided herein modulates one or more IL-1β activities. In some embodiments, the anti-IL-1β antibody provided herein is an antagonist antibody.

In one embodiment, the antibodies according to the disclosure are IL-1β antagonists. In another embodiment, the antibody or functional fragment comprising an antigen-binding portion binds the target protein IL-1β and decreases the binding of IL-1β to interleukin type 1 receptor (IL-1RI) to a basal level. In one aspect of this embodiment, the antibody or functional fragment reduces the amount of IL-1β that binds to IL-1RI. In a further aspect of this embodiment, the antibody or functional fragment completely prevents IL-1β from binding to IL-1RI. In a further embodiment, the antibody or functional fragment inhibits IL-1 signaling activation. An antibody that inhibits one or more of these IL-1β functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (e.g., or when a control antibody of irrelevant specificity is present). In some embodiments, an antibody that inhibits IL-1β activity effects such a statistically significant decrease by at least 10% of the measured parameter, by at least 50%, 80% or 90%, and in certain embodiments an antibody of the disclosure may inhibit greater than 95%, 98% or 99% of IL-1β functional activity.

In some embodiments, the anti-IL-1β antibody provided herein binds to IL-1β (e.g., human IL-1β) with a dissociation constant (K_{D}) of ≤1 μM, ≤200 nM, ≤100 nM, ≤50 nM, ≤20 nM, ≤10 nM, ≤1 nM, ≤0.1 nM, ≤0.05 nM, ≤0.02 nM, ≤0.01 nM, ≤0.001 nM (e.g. 20 pM, 22 pM, 64 pM, 70 pM, 180 pM, or 1.7 nM, e.g. 10⁻⁸ M or less, e.g. from 10⁻⁸M to 10⁻¹³ M, e.g., from 10⁻⁹M to 10⁻¹³M, e.g., from 10⁻¹⁰M to 10⁻¹³M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-IL-1β antibodies provide herein are those described in Section 7 below. Thus, in some embodiments, the antibody provided herein comprises one or more CDR sequences of any one of SEQ ID NOs: 13-102. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-IL-1β antibody is humanized. In some embodiments, the anti-IL-1β antibody comprises an acceptor human framework, e.g., a human immunoglobulin framework or a human consensus framework.

In some embodiments, the anti-IL-1β antibody provided herein comprises HCDR1, HCDR2, and HCDR3 as set forth in SEQ ID NO: 7. In some embodiments, the anti-IL-1β antibody provided herein comprises HCDR1, HCDR2, and HCDR3 as set forth in SEQ ID NO: 9. In some embodiments, the anti-IL-1β antibody provided herein comprises HCDR1, HCDR2, and HCDR3 as set forth in SEQ ID NO: 11. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the anti-IL-1β antibody provided herein comprises LCDR1, LCDR2, and LCDR3 as set forth in SEQ ID NO: 8. In some embodiments, the anti-IL-1β antibody provided herein comprises LCDR1, LCDR2, and LCDR3 as set forth in SEQ ID NO: 10. In some embodiments, the anti-IL-1β antibody provided herein comprises LCDR1, LCDR2, and LCDR3 as set forth in SEQ ID NO: 12. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the antibody or antigen binding fragment provided herein comprises an HCDR1, an HCDR2, and an HCDR3 as set forth in SEQ ID NO: 7, and a LCDR1, a LCDR2, and a LCDR3 as set forth in SEQ ID NO: 8. In some embodiments, the antibody or antigen binding fragment provided herein comprises an HCDR1, an HCDR2, and an HCDR3 as set forth in SEQ ID NO: 9, and a LCDR1, a LCDR2, and a LCDR3 as set forth in SEQ ID NO: 10. In some embodiments, the antibody or antigen binding fragment provided herein comprises an HCDR1, an HCDR2, and an HCDR3 as set forth in SEQ ID NO: 11, and a LCDR1, a LCDR2, and a LCDR3 as set forth in SEQ ID NO: 12. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to IL-1β comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 13, 19, 25, 31, 37, 43, 49, 55, 61, 67, 73, 79, 85, 91, and 97; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 14, 20, 26, 32, 38, 44, 50, 56, 62, 68, 74, 80, 86, 92, and 98, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 15, 21, 27, 33, 39, 45, 51, 57, 63, 69, 75, 81, 87, 93, and 99; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 16, 22, 28, 34, 40, 46, 52, 58, 64, 70, 76, 82, 88, 94, and 100; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 17, 23, 29, 35, 41, 47, 53, 59, 65, 71, 77, 83, 89, 95, and 101; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 18, 24, 30, 36, 42, 48, 54, 60, 66, 72, 78, 84, 90, 96, and 102. In some embodiments, the anti-IL-1β antibody is humanized. In some embodiments, the anti-IL-1β antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 13, the HCDR2 comprises the amino acid sequence of SEQ ID NO: 14, the HCDR3 comprises the amino acid sequence of SEQ ID NO: 15, the LCDR1 comprises the amino acid sequence of SEQ ID NO: 16, the LCDR2 comprises the amino acid sequence of SEQ ID NO: 17, and the LCDR3 comprises the amino acid sequence of SEQ ID NO: 18. In some specific embodiments, in the antibody or antigen binding fragment provided herein, the HCDR1 comprises the amino acid sequence of SEQ ID NO: 31, the HCDR2 comprises the amino acid sequence of SEQ ID NO: 32, the HCDR3 comprises the amino acid sequence of SEQ ID NO: 33, the LCDR1 comprises the amino acid sequence of SEQ ID NO: 34, the LCDR2 comprises the amino acid sequence of SEQ ID NO: 35, and the LCDR3 comprises the amino acid sequence of SEQ ID NO: 36. In some specific embodiments, in the antibody or antigen binding fragment provided herein, the HCDR1 comprises the amino acid sequence of SEQ ID NO: 49, the HCDR2 comprises the amino acid sequence of SEQ ID NO: 50, the HCDR3 comprises the amino acid sequence of SEQ ID NO: 51, the LCDR1 comprises the amino acid sequence of SEQ ID NO: 52, the LCDR2 comprises the amino acid sequence of SEQ ID NO: 53, and the LCDR3 comprises the amino acid sequence of SEQ ID NO: 54. In some specific embodiments, in the antibody or antigen binding fragment provided herein, the HCDR1 comprises the amino acid sequence of SEQ ID NO: 67, the HCDR2 comprises the amino acid sequence of SEQ ID NO: 68, the HCDR3 comprises the amino acid sequence of SEQ ID NO: 69, the LCDR1 comprises the amino acid sequence of SEQ ID NO: 70, the LCDR2 comprises the amino acid sequence of SEQ ID NO: 71, and the LCDR3 comprises the amino acid sequence of SEQ ID NO: 72. In some specific embodiments, in the antibody or antigen binding fragment provided herein, the HCDR1 comprises the amino acid sequence of SEQ ID NO: 85, the HCDR2 comprises the amino acid sequence of SEQ ID NO: 86, the HCDR3 comprises the amino acid sequence of SEQ ID NO: 87, the LCDR1 comprises the amino acid sequence of SEQ ID NO: 88, the LCDR2 comprises the amino acid sequence of SEQ ID NO: 89, and the LCDR3 comprises the amino acid sequence of SEQ ID NO: 90.

In some specific embodiments, in the antibody or antigen binding fragment provided herein, the HCDR1 comprises the amino acid sequence of SEQ ID NO: 19, the HCDR2 comprises the amino acid sequence of SEQ ID NO: 20, the HCDR3 comprises the amino acid sequence of SEQ ID NO: 21, the LCDR1 comprises the amino acid sequence of SEQ ID NO: 22, the LCDR2 comprises the amino acid sequence of SEQ ID NO: 23 and the LCDR3 comprises the amino acid sequence of SEQ ID NO: 24. In some specific embodiments, in the antibody or antigen binding fragment provided herein, the HCDR1 comprises the amino acid sequence of SEQ ID NO: 37, the HCDR2 comprises the amino acid sequence of SEQ ID NO: 38, the HCDR3 comprises the amino acid sequence of SEQ ID NO: 39, the LCDR1 comprises the amino acid sequence of SEQ ID NO: 40, the LCDR2 comprises the amino acid sequence of SEQ ID NO: 41 and the LCDR3 comprises the amino acid sequence of SEQ ID NO: 42. In some specific embodiments, in the antibody or antigen binding fragment provided herein, the HCDR1 comprises the amino acid sequence of SEQ ID NO: 55, the HCDR2 comprises the amino acid sequence of SEQ ID NO: 56, the HCDR3 comprises the amino acid sequence of SEQ ID NO: 57, the LCDR1 comprises the amino acid sequence of SEQ ID NO: 58, the LCDR2 comprises the amino acid sequence of SEQ ID NO: 59 and the LCDR3 comprises the amino acid sequence of SEQ ID NO: 60. In some specific embodiments, in the antibody or antigen binding fragment provided herein, the HCDR1 comprises the amino acid sequence of SEQ ID NO: 73, the HCDR2 comprises the amino acid sequence of SEQ ID NO: 74, the HCDR3 comprises the amino acid sequence of SEQ ID NO: 75, the LCDR1 comprises the amino acid sequence of SEQ ID NO: 76, the LCDR2 comprises the amino acid sequence of SEQ ID NO: 77 and the LCDR3 comprises the amino acid sequence of SEQ ID NO: 78. In some specific embodiments, in the antibody or antigen binding fragment provided herein, the HCDR1 comprises the amino acid sequence of SEQ ID NO: 91, the HCDR2 comprises the amino acid sequence of SEQ ID NO: 92, the HCDR3 comprises the amino acid sequence of SEQ ID NO: 93, the LCDR1 comprises the amino acid sequence of SEQ ID NO: 94, the LCDR2 comprises the amino acid sequence of SEQ ID NO: 95 and the LCDR3 comprises the amino acid sequence of SEQ ID NO: 96.

In some specific embodiments, in the antibody or antigen binding fragment provided herein, the HCDR1 comprises the amino acid sequence of SEQ ID NO: 25, the HCDR2 comprises the amino acid sequence of SEQ ID NO: 26, the HCDR3 comprises the amino acid sequence of SEQ ID NO: 27, the LCDR1 comprises the amino acid sequence of SEQ ID NO: 28, the LCDR2 comprises the amino acid sequence of SEQ ID NO: 29, and the LCDR3 comprises the amino acid sequence of SEQ ID NO: 30. In some specific embodiments, in the antibody or antigen binding fragment provided herein, the HCDR1 comprises the amino acid sequence of SEQ ID NO: 43, the HCDR2 comprises the amino acid sequence of SEQ ID NO: 44, the HCDR3 comprises the amino acid sequence of SEQ ID NO: 45, the LCDR1 comprises the amino acid sequence of SEQ ID NO: 46, the LCDR2 comprises the amino acid sequence of SEQ ID NO: 47, and the LCDR3 comprises the amino acid sequence of SEQ ID NO: 48. In some specific embodiments, in the antibody or antigen binding fragment provided herein, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some specific embodiments, in the antibody or antigen binding fragment provided herein, the HCDR1 comprises the amino acid sequence of SEQ ID NO: 79, the HCDR2 comprises the amino acid sequence of SEQ ID NO: 80, the HCDR3 comprises the amino acid sequence of SEQ ID NO: 81, the LCDR1 comprises the amino acid sequence of SEQ ID NO: 82, the LCDR2 comprises the amino acid sequence of SEQ ID NO: 83, and the LCDR3 comprises the amino acid sequence of SEQ ID NO: 84. In some specific embodiments, in the antibody or antigen binding fragment provided herein, the HCDR1 comprises the amino acid sequence of SEQ ID NO: 97, the HCDR2 comprises the amino acid sequence of SEQ ID NO: 98, the HCDR3 comprises the amino acid sequence of SEQ ID NO: 99, the LCDR1 comprises the amino acid sequence of SEQ ID NO: 100, the LCDR2 comprises the amino acid sequence of SEQ ID NO: 101, and the LCDR3 comprises the amino acid sequence of SEQ ID NO: 102.

In some embodiments, the antibody further comprises one or more framework regions of SEQ ID NOs: 13-102. In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody or antigen binding fragment provided herein comprises a VH comprising the amino acid sequence of SEQ ID NO: 7, and a VL comprising the amino acid sequence of SEQ ID NO: 8. In some embodiments, the antibody or antigen binding fragment provided herein comprises a VH comprising the amino acid sequence of SEQ ID NO: 9, and a VL comprising the amino acid sequence of SEQ ID NO: 10. In some embodiments, the antibody or antigen binding fragment provided herein comprises a VH comprising the amino acid sequence of SEQ ID NO: 11, and a VL comprising the amino acid sequence of SEQ ID NO: 12.

In certain embodiments, an antibody described herein or an antigen binding fragment thereof comprises amino acid sequences with certain percent identity relative to any antibody provided herein, for example, those described in Section 6 below.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and)(BLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of)(BLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the antibody provide herein contains substitutions (e.g., conservative substitutions), insertions, or deletions relative to the reference sequence, but the anti-IL-1β antibody comprising that sequence retains the ability to bind to IL-1β. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). In some embodiments, the anti-IL-1β antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 7, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 8. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 9, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 10. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 11, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 12. In all the embodiments described above, the antibodies bind to IL-1β.

In some embodiments, functional epitopes can be mapped, e.g., by combinatorial alanine scanning, to identify amino acids in the IL-1β protein that are necessary for interaction with anti-IL-1β antibodies provided herein. In some embodiments, conformational and crystal structure of anti-IL-1β antibody bound to IL-1β may be employed to identify the epitopes. In some embodiments, the present disclosure provides an antibody that specifically binds to the same epitope as any of the anti-IL-1β antibodies provided herein. For example, in some embodiments, the antibody or antigen binding fragment provided herein binds to the same epitope as an anti-IL-1β antibody comprising a VH comprising the amino acid sequence of SEQ ID NO: 7, and a VL comprising the amino acid sequence of SEQ ID NO: 8. In some embodiments, the antibody or antigen binding fragment provided herein binds to the same epitope as an anti-IL-1β antibody comprising a VH comprising the amino acid sequence of SEQ ID NO: 9, and a VL comprising the amino acid sequence of SEQ ID NO: 10. In some embodiments, the antibody or antigen binding fragment provided herein binds to the same epitope as an anti-IL-1β antibody comprising a VH comprising the amino acid sequence of SEQ ID NO: 11, and a VL comprising the amino acid sequence of SEQ ID NO: 12.

In some embodiments, provided herein is an anti-IL-1β antibody, or antigen binding fragment thereof, that specifically binds to IL-1β competitively with any one of the anti-IL-1β antibodies described herein. In some embodiments, the antibody or antigen binding fragment provided herein specifically binds to IL-1β competitively with an anti-IL-1β antibody comprising a VH comprising the amino acid sequence of SEQ ID NO: 7, and a VL comprising the amino acid sequence of SEQ ID NO: 8. In some embodiments, the antibody or antigen binding fragment provided herein specifically binds to IL-1β competitively with an anti-IL-1β antibody comprising a VH comprising the amino acid sequence of SEQ ID NO: 9, and a VL comprising the amino acid sequence of SEQ ID NO: 10. In some embodiments, the antibody or antigen binding fragment provided herein specifically binds to IL-1β competitively with an anti-IL-1β antibody comprising a VH comprising the amino acid sequence of SEQ ID NO: 11, and a VL comprising the amino acid sequence of SEQ ID NO: 12.

In some embodiments, provided herein is an IL-1β binding protein comprising any one of the anti-IL-1β antibodies described above. In some embodiments, the IL-1β binding protein is a monoclonal antibody, including a mouse, chimeric, humanized or human antibody. In some embodiments, the anti-IL-1β antibody is an antibody fragment, e.g., a scFv. In some embodiments, the IL-1β binding protein is a fusion protein comprising the anti-IL-1β antibody provided herein. In other embodiments, the IL-1β binding protein is a multispecific antibody comprising the anti-IL-1β antibody provided herein. Other exemplary IL-1β binding molecules are described in more detail in the following sections.

In some embodiments, the anti-IL-1β antibody or antigen binding protein according to any of the above embodiments may incorporate any of the features, singly or in combination, as described in Sections 5.2.2 to 5.2.7 below.

5.2.2. Antibody Fragments

As used herein, the term “antibody” also includes various antibody fragments thereof. Antibodies provided herein include, but are not limited to, immunoglobulin molecules and immunologically active portions of immunoglobulin molecules. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule. In some embodiments, the antibody is an IgG antibody. In some embodiments, the IgG antibody is an IgG1 antibody. In some embodiments, the IgG antibody is an IgG2, IgG3, or IgG4 antibody.

Variants and derivatives of antibodies include antibody functional fragments that retain the ability to bind to an antigen. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of, e.g., 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes); and a tetrabody (e.g., a tetramerized scFv, formed in a manner similar to a diabody, but in which four antigen-binding domains are created in a single complex; the four antigen binding domains may be directed towards the same or different epitopes).

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. For example, Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

5.2.3. Humanized Antibodies

The antibodies described herein include humanized antibodies. Humanized antibodies, such as the humanized antibodies disclosed herein can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology 28(4/5):489-498 (1991); Studnicka et al., Protein Engineering 7(6):805-814 (1994); and Roguska et al., PNAS 91:969-973 (1994)), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

In some embodiments, antibodies provided herein can be humanized antibodies that bind to IL-1β, including human IL-1β. For example, humanized antibodies of the present disclosure may comprise one or more CDRs set forth in SEQ ID NOs: 13-102. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., Nature 321:522-25 (1986); Riechmann et al., Nature 332:323-27 (1988); and Verhoeyen et al., Science 239:1534-36 (1988)), by substituting hypervariable region sequences for the corresponding sequences of a human antibody. In a specific embodiment, humanization of the antibody provided herein is performed as described in Section 6 below.

In some cases, the humanized antibodies are constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., FASEB J. 9:133-39 (1995)). In the technique of SDR grafting, only the SDR residues are grafted onto the human antibody framework (see, e.g., Kashmiri et al., Methods 36:25-34 (2005)).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., J. Immunol. 151:2296-308 (1993); and Chothia et al., J. Mol. Biol. 196:901-17 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA 89:4285-89 (1992); and Presta et al., J. Immunol. 151:2623-32 (1993)). In some cases, the framework is derived from the consensus sequences of the most abundant human subclasses, V_{L}6 subgroup I (V_{L}6I) and VH subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169:1119-25 (2002)).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-24 (2002)), Modeller (Sali and Blundell, J. Mol. Biol. 234:779-815 (1993)), and Swiss PDB Viewer (Guex and Peitsch, Electrophoresis 18:2714-23 (1997)). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol. 44:1986-98 (2007)).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol. 23:1105-16 (2005); Dufner et al., Trends Biotechnol. 24:523-29 (2006); Feldhaus et al., Nat. Biotechnol. 21:163-70 (2003); and Schlapschy et al., Protein Eng. Des. Sel. 17:847-60 (2004)).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol. 224:487-99 (1992)), or from the more limited set of target residues identified by Baca et al. J. Biol. Chem. 272:10678-84 (1997).

In FR shuffling, whole FRs are combined with the non-human CDRs instead of creating combinatorial libraries of selected residue variants (see, e.g., Dall'Acqua et al., Methods 36:43-60 (2005)). A one-step FR shuffling process may be used. Such a process has been shown to be efficient, as the resulting antibodies exhibited improved biochemical and physicochemical properties including enhanced expression, increased affinity, and thermal stability (see, e.g., Damschroder et al., Mol. Immunol. 44:3049-60 (2007)).

The “humaneering” method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The “human engineering” method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering 7:805-14 (1994); U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T-cell epitopes have been removed. Methods for making deimmunized antibodies have been described. See, e.g., Jones et al., Methods Mol Biol. 525:405-23 (2009), xiv, and De Groot et al., Cell. Immunol. 244:148-153(2006)). Deimmunized antibodies comprise T-cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

5.2.4. Antibody Variants

In some embodiments, amino acid sequence modification(s) of the antibodies that bind to IL-1β described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies that bind to IL-1β described herein, it is contemplated that variants of the antibodies that bind to IL-1β described herein can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody.

### Chemical Modifications

In some embodiments, the antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the antibody provided herein is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108; and WO 2004/056312, and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

The binding molecules comprising an antibody provided herein are further provided with bisected oligosaccharides, e.g., in which a biantennary oligosaccharide attached to the Fc region is bisected by GlcNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

In molecules that comprise the present antibody and an Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the present application contemplates variants that possesses some but not all effector functions, which make it a desirable candidate for applications in which the half-life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcγR binding (hence likely lacking ADCC activity) but retains FcRn binding ability. Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Intl. Immunol. 18(12):1759-1769 (2006)).

Binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, a variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Binding molecules with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

### Substitutions, Deletions, or Insertions

Variations may be a substitution, deletion, or insertion of one or more codons encoding the antibody or polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in Table 2 below.

TABLE 2
Amino Acid Substitutions

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. More detailed description regarding affinity maturation is provided in the section below.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant antibody sequences provided herein, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

### Fc Mutations

To facilitate the formation of a heterodimer between the two heavy chains, e.g., one with a fusion of the anti-IL-1β antibody or antigen-binding fragment thereof and one without, or one containing the Fc for the anti-IL-1β arm and one for the tissue target arm, heterodimeric mutations introduced into the Fc of the two heavy chains. Examples of such Fc mutations include, but are not limited to, the Zymework mutations (see, e.g., U.S. Pat. No. 10,457,742) and the “knob in hole” mutations (see, e.g., Ridgway et al., Protein Eng., 9(7): 617-621, 1996). Other heterodimer mutations can also be used in the present disclosure. In some embodiment, a modified CH3 as described herein is used to facilitate the formation of a heterodimer between the two heavy chains.

In a specific embodiment, each of the two heavy chains of the antibody comprises one or more heterodimeric mutation(s) or one or more knob and hole mutation(s). In a specific embodiment, the one or more heterodimeric mutation(s) is in the CH3 domain.

In certain embodiments, the Fc region of the antibody or antigen binding fragment thereof contains substitutions that alter the binding of the antibody or antigen binding fragment thereof to neonatal Fc receptor (FcRn). In certain embodiments, the Fc region of the antibody or antigen binding fragment thereof contains substitutions that enhance the binding of the antibody or antigen binding fragment thereof to neonatal Fc receptor (FcRn). In certain embodiments, the Fc region of the antibody or antigen binding fragment thereof contains substitutions that enhance the binding of the antibody or antigen binding fragment thereof to neonatal Fc receptor (FcRn) at pH˜6. In certain embodiments, the Fc region of the antibody or antigen binding fragment thereof contains substitutions that enhance the binding of the antibody or antigen binding fragment thereof to neonatal Fc receptor (FcRn) at pH˜6, thereby enhancing FcRn-mediated endosomal recycling. In certain embodiments, the Fc region of the antibody or antigen binding fragment thereof contains substitutions that enhance the binding of the antibody or antigen binding fragment thereof to neonatal Fc receptor (FcRn) at pH˜6, thereby leading to longer serum exposure. In certain embodiments, the Fc region of the antibody or antigen binding fragment thereof contains substitutions that enhance the binding at an acidic pH. In certain embodiments, the Fc region of the antibody or antigen binding fragment thereof has the M252Y/S254T/T256E (YTE) mutations, wherein the numbering of amino acid residues is according to the EU index.

5.2.5. In vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37 (2002); and Bradbury and Marks, J. Immunol. Methods 290:29-49 (2004).

In a yeast display system (see, e.g., Boder et al., Nat. Biotech. 15:553-57 (1997); and Chao et al., Nat. Protocols 1:755-68 (2006)), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single chain antibody (e.g., scFv). Expression has been shown to correlate with the stability of the displayed protein, and thus antibodies can be selected for improved stability as well as affinity (see, e.g., Shusta et al., J. Mol. Biol. 292:949-56 (1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., Gene 342:211-18 (2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., Nucleic Acids Res. 34:e127 (2006)). In mRNA display, a covalent bond between antibody and mRNA is established using puromycin as an adaptor molecule (Wilson et al., Proc. Natl. Acad. Sci. USA 98:3750-55 (2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003); U.S. Pat. Nos. 5,565,332 and 6,989,250). Alternative techniques target hypervariable loops extending into framework-region residues (see, e.g., Bond et al., J. Mol. Biol. 348:699-709 (2005)) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, antibodies can be immobilized onto solid supports, columns, pins, or cellulose/poly (vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, Nature Biotechnology 23:1105-16 (2005); Quiroz and Sinclair, Revista Ingeneria Biomedia 4:39-51 (2010); and references therein.

5.2.6. Modifications of Antibodies

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide as described above (see, e.g., Beck et al., Curr. Pharm. Biotechnol. 9:482-501 (2008); and Walsh, Drug Discov. Today 15:773-80 (2010)), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337. The antibody that binds to IL-1β of the disclosure may also be genetically fused or conjugated to one or more immunoglobulin constant regions or portions thereof (e.g., Fc) to extend half-life and/or to impart known Fc-mediated effector functions.

The antibody that binds to IL-1β of the present disclosure may also be modified to form chimeric molecules comprising the antibody that binds to IL-1β fused to another, heterologous polypeptide or amino acid sequence, for example, an epitope tag (see, e.g., Terpe, Appl. Microbiol. Biotechnol. 60:523-33 (2003)) or the Fc region of an IgG molecule (see, e.g., Aruffo, Antibody Fusion Proteins 221-42 (Chamow and Ashkenazi eds., 1999)).

Also provided herein are fusion proteins comprising the antibody that binds to IL-1β of the disclosure and a heterologous polypeptide. In some embodiments, the heterologous polypeptide to which the antibody is genetically fused or chemically conjugated is useful for targeting the antibody to cells having cell surface-expressed IL-1β.

Also provided herein are panels of antibodies that bind to an IL-1β antigen. In specific embodiments, the panels of antibodies have different association rates, different dissociation rates, different affinities for an IL-1β antigen, and/or different specificities for an IL-1β antigen. In some embodiments, the panels comprise or consist of about 10 to about 1000 antibodies or more. Panels of antibodies can be used, for example, in 96-well or 384-well plates, for assays such as ELISAs.

5.2.7. Other Binding Molecules Comprising the Antibodies

In another aspect, provided herein is a binding molecule comprising an anti-IL-1β antibody provided herein. In some embodiments, an antibody against IL-1β provided herein is part of other binding molecules. Exemplary binding molecules of the present disclosure are described herein.

### Fusion Protein

In various embodiments, the antibody provided herein can be genetically fused or chemically conjugated to another agent, for example, protein-based entities. The antibody may be chemically-conjugated to the agent, or otherwise non-covalently conjugated to the agent. The agent can be a peptide or antibody (or a fragment thereof).

Thus, in some embodiments, provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof. In particular, provided herein are fusion proteins comprising an antigen binding fragment of the antibody provided herein (e.g., CDR1, CDR2, and/or CDR3) and a heterologous protein, polypeptide, or peptide.

Moreover, antibodies provided herein can be fused to marker or “tag” sequences, such as a peptide, to facilitate purification. In specific embodiments, the marker or tag amino acid sequence is a hexa-histidine peptide, hemagglutinin (“HA”) tag, and “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., Immunol. Rev. 62:119-58 (1982); U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-39 (1991); Traunecker et al., Nature, 331:84-86 (1988); Zheng et al., J. Immunol. 154:5590-600 (1995); and Vil et al., Proc. Natl. Acad. Sci. USA 89:11337-41 (1992)).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and U.S. Pat. No. 5,837,458; Patten et al., Curr. Opinion Biotechnol. 8:724-33 (1997); Harayama, Trends Biotechnol. 16(2):76-82 (1998); Hansson et al., J. Mol. Biol. 287:265-76 (1999); and Lorenzo and Blasco, Biotechniques 24(2):308-13 (1998)). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

In some embodiments, an antibody provided herein is conjugated to a second antibody to form an antibody heteroconjugate.

In various embodiments, the antibody is genetically fused to the agent. Genetic fusion may be accomplished by placing a linker (e.g., a polypeptide) between the antibody and the agent. The linker may be a flexible linker.

In various embodiments, the antibody is genetically conjugated to a therapeutic molecule, with a hinge region linking the antibody to the therapeutic molecule.

Also provided herein are methods for making the various fusion proteins provided herein. The various methods described in Section 5.4 may also be utilized to make the fusion proteins provided herein.

In a specific embodiment, the fusion protein provided herein is recombinantly expressed. Recombinant expression of a fusion protein provided herein may require construction of an expression vector containing a polynucleotide that encodes the protein or a fragment thereof. Once a polynucleotide encoding a protein provided herein or a fragment thereof has been obtained, the vector for the production of the molecule may be produced by recombinant DNA technology using techniques well-known in the art. Thus, methods for preparing a protein by expressing a polynucleotide containing an encoding nucleotide sequence are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a fusion protein provided herein, or a fragment thereof, or a CDR, operably linked to a promoter.

The expression vector can be transferred to a host cell by conventional techniques and the transfected cells are then cultured by conventional techniques to produce a fusion protein provided herein. Thus, also provided herein are host cells containing a polynucleotide encoding a fusion protein provided herein or fragments thereof operably linked to a heterologous promoter.

A variety of host-expression vector systems may be utilized to express the fusion protein provided herein. Such host-expression systems represent vehicles by which the coding sequences of interest may be produced and subsequently purified, but also represent cells which may, when transformed or transfected with the appropriate nucleotide coding sequences, express a fusion protein provided herein in situ. These include but are not limited to microorganisms such as bacteria (e.g., E. coli and B. subtilis) transformed with recombinant bacteriophage DNA, plasmid DNA or cosmid DNA expression vectors containing coding sequences; yeast (e.g., Saccharomyces Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant fusion protein. For example, mammalian cells such as Chinese hamster ovary cells (CHO), in conjunction with a vector such as the major intermediate early gene promoter element from human cytomegalovirus is an effective expression system for antibodies or variants thereof. In a specific embodiment, the expression of nucleotide sequences encoding the fusion proteins provided herein is regulated by a constitutive promoter, inducible promoter or tissue specific promoter.

In bacterial systems, a number of expression vectors may be advantageously selected depending upon the use intended for the fusion protein being expressed. For example, when a large quantity of such a fusion protein is to be produced, for the generation of pharmaceutical compositions of a fusion protein, vectors which direct the expression of high levels of fusion protein products that are readily purified may be desirable. Such vectors include, but are not limited to, the E. coli expression vector pUR278 (Ruther et al., EMBO 12:1791 (1983)), in which the coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the fusion protein in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression can be utilized. For example, cell lines which stably express the fusion proteins may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion protein. Such engineered cell lines may be particularly useful in screening and evaluation of compositions that interact directly or indirectly with the binding molecule.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8-17 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., J. Mol. Biol. 150:1 (1981), which are incorporated by reference herein in their entireties.

The expression level of a fusion protein can be increased by vector amplification (for a review, see Bebbington and Hentschel) The use of vectors based on gene amplification for the expression of cloned genes in mammalian cells in DNA cloning, Vol. 3 (Academic Press, New York, 1987)). When a marker in the vector system expressing a fusion protein is amplifiable, increase in the level of inhibitor present in culture of host cell will increase the number of copies of the marker gene. Since the amplified region is associated with the fusion protein gene, production of the fusion protein will also increase (Crouse et al., Mol. Cell. Biol. 3:257 (1983)).

The host cell may be co-transfected with multiple expression vectors provided herein. The vectors may contain identical selectable markers which enable equal expression of respective encoding polypeptides. Alternatively, a single vector may be used which encodes, and is capable of expressing multiple polypeptides. The coding sequences may comprise cDNA or genomic DNA.

Once a fusion protein provided herein has been produced by recombinant expression, it may be purified by any method known in the art for purification of a polypeptide (e.g., an immunoglobulin molecule), for example, by chromatography (e.g., ion exchange, affinity, particularly by affinity for the specific antigen after Protein A, sizing column chromatography, and Kappa select affinity chromatography), centrifugation, differential solubility, or by any other standard technique for the purification of proteins. Further, the fusion protein molecules provided herein can be fused to heterologous polypeptide sequences described herein or otherwise known in the art to facilitate purification.

### Immunoconjugates

In some embodiments, the present disclosure also provides immunoconjugates comprising any of the anti-IL-1β antibodies described herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

The immunoconjugates or ADCs herein contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

In other embodiments, antibodies provided herein are conjugated or recombinantly fused, e.g., to a diagnostic molecule. Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, 225Acγ-emitting, Auger-emitting, β-emitting, an alpha-emitting or positron-emitting radioactive isotope.

### 5.3. Polynucleotides

In certain embodiments, the disclosure provides polynucleotides that encode the present antibodies that bind to IL-1β and fusion proteins comprising the antibodies that bind to IL-1β described herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the antibody that binds IL-1β of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the antibody that binds IL-1β of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five-point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In certain embodiments, a polynucleotide is isolated. In certain embodiments, a polynucleotide is substantially pure.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, Va., CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-K1SV (Lonza Biologics, Walkersville, Md.), CHO-K1 (ATCC CRL-61) or DG44.

### 5.4. Preparation of Antibodies and Method of Making

Methods of preparing antibodies have been described. See, e.g., Els Pardon et al, Nature Protocol, 9(3): 674 (2014). Antibodies (such as scFv fragments) may be obtained using methods known in the art such as by immunizing a Camelid species (such as camel or llama) and obtaining hybridomas therefrom, or by cloning a library of antibodies using molecular biology techniques known in the art and subsequent selection by ELISA with individual clones of unselected libraries or by using phage display.

Antibodies provided herein may be produced by culturing cells transformed or transfected with a vector containing an antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-IL-1β antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the anti-IL-1β antibody may be chemically synthesized separately and combined using chemical or enzymatic methods to produce the desired anti-IL-1β antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

### Polyclonal Antibodies

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and Ware independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

For example, the animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, an appropriate host animal is immunized to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Goding, Monoclonal Antibodies: Principles and Practice, Academic Press (1986), pp. 59-103. Immortalized cell lines are usually transformed mammalian cells. The hybridoma cells thus prepared are seeded and grown in a suitable culture medium that preferably contains one or more substances that inhibit the growth or survival of the unfused, parental myeloma cells. Preferred immortalized myeloma cells are those that fuse efficiently, support stable high-level production of antibody by the selected antibody-producing cells, and are sensitive to a medium such as HAT medium.

Culture medium in which hybridoma cells are growing is assayed for production of monoclonal antibodies directed against the antigen. The culture medium in which the hybridoma cells are cultured can be assayed for the presence of monoclonal antibodies directed against the desired antigen. Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991). Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence (U.S. Pat. No. 4,816,567; Morrison, et al., Proc. Natl Acad. Sci. USA, 81:6851 (1984)), or by covalently joining to the coding sequence all or part of the coding sequence for a non-immunoglobulin polypeptide. Such non-immunoglobulin polypeptides can be substituted to create a chimeric bivalent antibody comprising one antigen-combining site having specificity for an antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl mercaptobutyrimidate.

### Recombinant Production in Prokaryotic Cells

Polynucleic acid sequences encoding the antibodies of the present disclosure can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the present antibody by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the -galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20: 269 (1980)) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence can be substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the antibodies according to the present disclosure can occur in the cytoplasm of the host cell, and therefore does not require the presence of secretion signal sequences within each cistron. Certain host strains (e.g., the E. coli trxB⁻ strains) provide cytoplasm conditions that are favorable for disulfide bond formation, thereby permitting proper folding and assembly of expressed protein subunits.

Prokaryotic host cells suitable for expressing the antibodies of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures and pHs.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods 263:133-147 (2002)). A variety of other inducers may be used, according to the vector construct employed, as is known in the art.

The expressed antibodies of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. To improve the production yield and quality of the antibodies of the present disclosure, various fermentation conditions can be modified. For example, the chaperone proteins have been demonstrated to facilitate the proper folding and solubility of heterologous proteins produced in bacterial host cells. Chen et al. J Bio Chem 274:19601-19605 (1999); U.S. Pat. Nos. 6,083,715; 6,027,888; Bothmann and Pluckthun, J. Biol. Chem. 275:17100-17105 (2000); Ramm and Pluckthun, J. Biol. Chem. 275:17106-17113 (2000); Arie et al., Mol. Microbiol. 39:199-210 (2001).

To minimize proteolysis of expressed heterologous proteins (especially those that are proteolytically sensitive), certain host strains deficient for proteolytic enzymes can be used for the present invention, as described in, for example, U.S. Pat. Nos. 5,264,365; 5,508,192; Hara et al., Microbial Drug Resistance, 2:63-72 (1996). E. coli strains deficient for proteolytic enzymes and transformed with plasmids overexpressing one or more chaperone proteins may be used as host cells in the expression system encoding the antibodies of the present application.

The antibodies produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75. Protein A immobilized on a solid phase for example can be used in some embodiments for immunoaffinity purification of binding molecules of the present disclosure. The solid phase to which Protein A is immobilized is preferably a column comprising a glass or silica surface, more preferably a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally the antibodies of interest is recovered from the solid phase by elution.

### Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate host cell when wild-type DHFR is employed is the Chinese hamster ovary (CHO) cell line deficient in DHFR activity. Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the antibodies of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells can be transformed with the above-described expression or cloning vectors for antibodies production and cultured in conventional nutrient media modified as appropriate for inducing promoters, selecting transformants, or amplifying the genes encoding the desired sequences.

The host cells used to produce the antibodies of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibodies can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography. In some embodiment, the protein composition prepared from the cells can be purified using an AKTA chromatography system. The matrix to which the affinity ligand is attached is most often agarose, but other matrices are available. Mechanically stable matrices such as controlled pore glass or poly (styrene-divinyl) benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture comprising the antibody of interest and contaminants may be subjected to low pH hydrophobic interaction chromatography.

### 5.5. Pharmaceutical Compositions

In one aspect, the present disclosure further provides pharmaceutical compositions comprising at least one antibody or antigen binding fragment thereof of the present disclosure. In some embodiments, a pharmaceutical composition comprises therapeutically effective amount of an antibody or antigen binding fragment thereof provided herein and a pharmaceutically acceptable excipient.

Pharmaceutical compositions comprising an antibody or antigen binding fragment thereof are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody or antigen binding fragment thereof of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof as described herein, including, but not limited to, encapsulation in liposomes, microparticles, microcapsules, recombinant cells capable of expressing the antibody or antigen binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6 Methods of Using the Antibodies

In one aspect, provided herein is a method of attenuating an activity of IL-1β in a cell, comprising exposing the cell to an effective amount of an antibody provided herein.

In some embodiments, the antibody provided herein inhibits IL-1β signaling pathway. In some embodiments, the antibody provided herein inhibits IL-1β bioactivity. In some embodiments, the antibody provided herein inhibits IL-6 production. In some embodiments, the antibody provided herein inhibits CXCL5 production. In some embodiments, the antibody provided herein inhibits G-CSF production. In some embodiments, the antibody provided herein inhibits IL-6, CXCL5, and G-CSF production. In some embodiments, the antibody provided herein inhibits IL-1β bioactivity in human fibroblasts. In some embodiments, the antibody provided herein inhibits IL-6, CXCL5, and G-CSF production in human fibroblasts. In some embodiments, the antibody provided herein inhibits IL-1β bioactivity in human fibroblasts. In some embodiments, the antibody provided herein inhibits IL-1β bioactivity in human peripheral blood mononuclear cell (PBMC) samples. In some embodiments, the antibody provided herein inhibits IL-1β bioactivity in human whole blood samples.

In some embodiments, the antibody provided herein crossreact with cynomolgus IL-1β. In some embodiments, the antibody provided herein inhibits IL-1β bioactivity in cynomolgus monkey fibroblasts.

In some embodiments, the antibody provided herein attenuates an IL-1β activity by at least about 10%. In some embodiments, the antibody provided herein attenuates an IL-1β activity by at least about 20%. In some embodiments, the antibody provided herein attenuates an IL-1β activity by at least about 30%. In some embodiments, the antibody provided herein attenuates an IL-1β activity by at least about 40%. In some embodiments, the antibody provided herein attenuates an IL-1β activity by at least about 50%. In some embodiments, the antibody provided herein attenuates an IL-1β activity by at least about 60%. In some embodiments, the antibody provided herein attenuates an IL-1β activity by at least about 70%. In some embodiments, the antibody provided herein attenuates an IL-1β activity by at least about 80%. In some embodiments, the antibody provided herein attenuates an IL-1β activity by at least about 90%. In some embodiments, the antibody provided herein attenuates an IL-1β activity by at least about 95%. In some embodiments, the antibody provided herein attenuates an IL-1β activity by at least about 98%. In some embodiments, the antibody provided herein attenuates an IL-1β activity by about 100%. In certain embodiments, the antibody described herein can attenuate (e.g., partially attenuate) an IL-1β activity by at least about 15% to about 65%. In certain embodiments, the antibody described herein can attenuate (e.g., partially attenuate) an IL-1β activity by at least about 20% to about 65%. In certain embodiments, the antibody described herein can attenuate (e.g., partially attenuate) an IL-1β activity by at least about 30% to about 65%.

A non-limiting example of an IL-1β activity is IL-1β mediated signaling. Thus, in certain embodiments, provided herein is a method of attenuating (e.g., partially attenuating) IL-1β mediated signaling in a cell, comprising exposing the cell to an effective amount of an antibody or antigen binding fragment thereof provided herein.

In some embodiments, the antibody provided herein attenuates IL-1β mediated signaling by at least about 10%. In some embodiments, the antibody provided herein attenuates IL-1β mediated signaling by at least about 20%. In some embodiments, the antibody provided herein attenuates IL-1β mediated signaling by at least about 30%. In some embodiments, the antibody provided herein attenuates IL-1β mediated signaling by at least about 40%. In some embodiments, the antibody provided herein attenuates IL-1β mediated signaling by at least about 50%. In some embodiments, the antibody provided herein attenuates IL-1β mediated signaling by at least about 60%. In some embodiments, the antibody provided herein attenuates IL-1β mediated signaling by at least about 70%. In some embodiments, the antibody provided herein attenuates IL-1β mediated signaling by at least about 80%. In some embodiments, the antibody provided herein attenuates IL-1β mediated signaling by at least about 90%. In some embodiments, the antibody provided herein attenuates IL-1β mediated signaling by at least about 95%. In some embodiments, the antibody provided herein attenuates IL-1β mediated signaling by at least about 98%. In some embodiments, the antibody provided herein attenuates IL-1β mediated signaling by about 100%. In certain embodiments, the antibody described herein can attenuate (e.g., partially attenuate) IL-1β mediated signaling by at least about 15% to about 65%. In certain embodiments, the antibody described herein can attenuate (e.g., partially attenuate) IL-1β mediated signaling by at least about 20% to about 65%. In certain embodiments, the antibody described herein can attenuate (e.g., partially attenuate) IL-1β mediated signaling by at least about 30% to about 65%.

In some embodiments, the antibody provided herein attenuates IL-1β induced production of IL-6 by at least about 10%. In some embodiments, the antibody provided herein attenuates IL-1β induced production of IL-6 by at least about 20%. In some embodiments, the antibody provided herein attenuates IL-1β induced production of IL-6 by at least about 30%. In some embodiments, the antibody provided herein attenuates IL-1β induced production of IL-6 by at least about 40%. In some embodiments, the antibody provided herein attenuates IL-1β induced production of IL-6 by at least about 50%. In some embodiments, the antibody provided herein attenuates IL-1β induced production of IL-6 by at least about 60%. In some embodiments, the antibody provided herein attenuates IL-1β induced production of IL-6 by at least about 70%. In some embodiments, the antibody provided herein attenuates IL-1β induced production of IL-6 by at least about 80%. In some embodiments, the antibody provided herein attenuates IL-1β induced production of IL-6 by at least about 90%. In some embodiments, the antibody provided herein attenuates IL-1β induced production of IL-6 by at least about 95%. In some embodiments, the antibody provided herein attenuates IL-1β induced production of IL-6 by at least about 98%. In some embodiments, the antibody provided herein attenuates IL-1β induced production of IL-6 by about 100%. In certain embodiments, the antibody described herein can attenuate (e.g., partially attenuate) IL-1β induced production of IL-6 by at least about 15% to about 65%. In certain embodiments, the antibody described herein can attenuate (e.g., partially attenuate) IL-1β induced production of IL-6 by at least about 20% to about 65%. In certain embodiments, the antibody described herein can attenuate (e.g., partially attenuate) IL-1β induced production of IL-6 by at least about 30% to about 65%.

In some embodiments, the antibody provided herein attenuates IL-1β induced production of CXCL5 by at least about 10%. In some embodiments, the antibody provided herein attenuates IL-1β induced production of CXCL5 by at least about 20%. In some embodiments, the antibody provided herein attenuates IL-1β induced production of CXCL5 by at least about 30%. In some embodiments, the antibody provided herein attenuates IL-1β induced production of CXCL5 by at least about 40%. In some embodiments, the antibody provided herein attenuates IL-1β induced production of CXCL5 by at least about 50%. In some embodiments, the antibody provided herein attenuates IL-1β induced production of CXCL5 by at least about 60%. In some embodiments, the antibody provided herein attenuates IL-1β induced production of CXCL5 by at least about 70%. In some embodiments, the antibody provided herein attenuates IL-1β induced production of CXCL5 by at least about 80%. In some embodiments, the antibody provided herein attenuates IL-1β induced production of CXCL5 by at least about 90%. In some embodiments, the antibody provided herein attenuates IL-1β induced production of CXCL5 by at least about 95%. In some embodiments, the antibody provided herein attenuates IL-1β induced production of CXCL5 by at least about 98%. In some embodiments, the antibody provided herein attenuates IL-1β induced production of CXCL5 by about 100%. In certain embodiments, the antibody described herein can attenuate (e.g., partially attenuate) IL-1β induced production of CXCL5 by at least about 15% to about 65%. In certain embodiments, the antibody described herein can attenuate (e.g., partially attenuate) IL-1β induced production of CXCL5 by at least about 20% to about 65%. In certain embodiments, the antibody described herein can attenuate (e.g., partially attenuate) IL-1β induced production of CXCL5 by at least about 30% to about 65%.

In some embodiments, the antibody provided herein attenuates IL-1β induced production of G-CSF by at least about 10%. In some embodiments, the antibody provided herein attenuates IL-1β induced production of G-CSF by at least about 20%. In some embodiments, the antibody provided herein attenuates IL-1β induced production of G-CSF by at least about 30%. In some embodiments, the antibody provided herein attenuates IL-1β induced production of G-CSF by at least about 40%. In some embodiments, the antibody provided herein attenuates IL-1β induced production of G-CSF by at least about 50%. In some embodiments, the antibody provided herein attenuates IL-1β induced production of G-CSF by at least about 60%. In some embodiments, the antibody provided herein attenuates IL-1β induced production of G-CSF by at least about 70%. In some embodiments, the antibody provided herein attenuates IL-1β induced production of G-CSF by at least about 80%. In some embodiments, the antibody provided herein attenuates IL-1β induced production of G-CSF by at least about 90%. In some embodiments, the antibody provided herein attenuates IL-1β induced production of G-CSF by at least about 95%. In some embodiments, the antibody provided herein attenuates IL-1β induced production of G-CSF by at least about 98%. In some embodiments, the antibody provided herein attenuates IL-1β induced production of G-CSF by about 100%. In certain embodiments, the antibody described herein can attenuate (e.g., partially attenuate) IL-1β induced production of G-CSF by at least about 15% to about 65%. In certain embodiments, the antibody described herein can attenuate (e.g., partially attenuate) IL-1β induced production of G-CSF by at least about 20% to about 65%. In certain embodiments, the antibody described herein can attenuate (e.g., partially attenuate) IL-1β induced production of G-CSF by at least about 30% to about 65%.

In some embodiments, the antibodies provided herein attenuate IL-1β binding to its at least one of its receptors.

Another non-limiting example of an IL-1β activity is binding to IL-1R1. Thus, in certain embodiments, provided herein is a method of attenuating (e.g., partially attenuating) the binding of IL-1β to IL-1R1, comprising exposing a cell to an effective amount of an antibody or antigen binding fragment thereof provided herein.

In some embodiments, the antibody provided herein attenuates the binding of IL-1β to IL-1R1 by at least about 10%. In some embodiments, the antibody provided herein attenuates the binding of IL-1β to IL-1R1 by at least about 20%. In some embodiments, the antibody provided herein attenuates the binding of IL-1β to IL-1R1 by at least about 30%. In some embodiments, the antibody provided herein attenuates the binding of IL-1β to IL-1R1 by at least about 40%. In some embodiments, the antibody provided herein attenuates the binding of IL-1β to IL-1R1 by at least about 50%. In some embodiments, the antibody provided herein attenuates the binding of IL-1β to IL-1R1 by at least about 60%. In some embodiments, the antibody provided herein attenuates the binding of IL-1β to IL-1R1 by at least about 70%. In some embodiments, the antibody provided herein attenuates the binding of IL-1β to IL-1R1 by at least about 80%. In some embodiments, the antibody provided herein attenuates the binding of IL-1β to IL-1R1 by at least about 90%. In some embodiments, the antibody provided herein attenuates the binding of IL-1β to IL-1R1 by at least about 95%. In some embodiments, the antibody provided herein attenuates the binding of IL-1β to IL-1R1 by at least about 98%. In some embodiments, the antibody provided herein attenuates the binding of IL-1β to IL-1R1 by about 100%. In certain embodiments, the antibody described herein can attenuate (e.g., partially attenuate) the binding of IL-1β to IL-1R1 by at least about 15% to about 65%. In certain embodiments, the antibody described herein can attenuate (e.g., partially attenuate) the binding of IL-1β to IL-1R1 by at least about 20% to about 65%. In certain embodiments, the antibody described herein can attenuate (e.g., partially attenuate) the binding of IL-1β to IL-1R1 by at least about 30% to about 65%.

Yet another non-limiting example of an IL-1β activity is signaling mediated by IL-1R1. Thus, in certain embodiments, provided herein is a method of attenuating (e.g., partially attenuating) IL-1R1 mediated signaling in a cell, comprising exposing the cell to an effective amount of an antibody or antigen binding fragment thereof provided herein.

In some embodiments, the antibody provided herein attenuates IL-1R1 mediated signaling by at least about 10%. In some embodiments, the antibody provided herein attenuates IL-1R1 mediated signaling by at least about 20%. In some embodiments, the antibody provided herein attenuates IL-1R1 mediated signaling by at least about 30%. In some embodiments, the antibody provided herein attenuates IL-1R1 mediated signaling by at least about 40%. In some embodiments, the antibody provided herein attenuates IL-1R1 mediated signaling by at least about 50%. In some embodiments, the antibody provided herein attenuates IL-1R1 mediated signaling by at least about 60%. In some embodiments, the antibody provided herein attenuates IL-1R1 mediated signaling by at least about 70%. In some embodiments, the antibody provided herein attenuates IL-1R1 mediated signaling by at least about 80%. In some embodiments, the antibody provided herein attenuates IL-1R1 mediated signaling by at least about 90%. In some embodiments, the antibody provided herein attenuates IL-1R1 mediated signaling by at least about 95%. In some embodiments, the antibody provided herein attenuates IL-1R1 mediated signaling by at least about 98%. In some embodiments, the antibody provided herein attenuates IL-1R1 mediated signaling by about 100%. In certain embodiments, the antibody described herein can attenuate (e.g., partially attenuate) IL-1R1 mediated signaling by at least about 15% to about 65%. In certain embodiments, the antibody described herein can attenuate (e.g., partially attenuate) IL-1R1-mediated signaling by at least about 20% to about 65%. In certain embodiments, the antibody described herein can attenuate (e.g., partially attenuate) IL-1R1 mediated signaling by at least about 30% to about 65%.

In some embodiments, the antibody provided herein inhibits the IL-1 signaling pathway initiation and progression by at least about 10%. In some embodiments, the antibody provided herein inhibits the IL-1 signaling pathway initiation and progression by at least about 20%. In some embodiments, the antibody provided herein inhibits the IL-1 signaling pathway initiation and progression by at least about 30%. In some embodiments, the antibody provided herein inhibits the IL-1 signaling pathway initiation and progression by at least about 40%. In some embodiments, the antibody provided herein inhibits the IL-1 signaling pathway initiation and progression by at least about 50%. In some embodiments, the antibody provided herein inhibits the IL-1 signaling pathway initiation and progression by at least about 60%. In some embodiments, the antibody provided herein inhibits the IL-1 signaling pathway initiation and progression by at least about 70%. In some embodiments, the antibody provided herein inhibits the IL-1 signaling pathway initiation and progression by at least about 80%. In some embodiments, the antibody provided herein inhibits the IL-1 signaling pathway initiation and progression by at least about 90%. In some embodiments, the antibody provided herein inhibits the IL-1 signaling pathway initiation and progression by at least about 95%. In some embodiments, the antibody provided herein inhibits the IL-1 signaling pathway initiation and progression by at least about 98%. In some embodiments, the antibody provided herein inhibits the IL-1 signaling pathway initiation and progression by about 100%. In certain embodiments, the antibody described herein can attenuate (e.g., partially attenuate) the IL-1 signaling pathway initiation and progression by at least about 15% to about 65%. In certain embodiments, the antibody described herein can attenuate (e.g., partially attenuate) the IL-1 signaling pathway initiation and progression by at least about 20% to about 65%. In certain embodiments, the antibody described herein can attenuate (e.g., partially attenuate) the IL-1 signaling pathway initiation and progression by at least about 30% to about 65%.

In another aspect, provided herein is a method of treating a disease or disorder in a subject comprising administering to the subject an effective amount of an antibody or antigen binding fragment thereof provided herein. In one embodiment, the disease or disorder is IL-1β-mediated disease or disorder. In one embodiment, the disease or disorder is IL-1R1-mediated disease or disorder. Also provided herein is a method of treatment of a disease or disorder, wherein the subject is administered one or more therapeutic agents in combination with the antibody or antigen-binding fragment thereof provided herein.

The disclosure also relates to methods of using the antibodies provided herein to inhibit, i.e., antagonize, function of IL-1β in order to inhibit IL-1 signaling pathway activation and thereby regulate inflammation, resulting in the treatment of a pathological disorder, such as cancer. In some embodiments, the cancer is lung cancer. In some embodiments, the cancer is renal cell cancer.

In some embodiments, provided herein is a method for treating an IL-1β mediated disease or disorder in a subject in need thereof, comprising administering to the subject an effective amount of an isolated IL-1β antibody or antigen binding fragment thereof as described herein. In some embodiments, provided herein is a method for treating an inflammatory disease or disorder in a subject in need thereof, comprising administering to the subject an effective amount of an isolated IL-1β antibody or antigen binding fragment thereof as described herein. In some embodiments, the IL-1β mediated disease or disorder is cancer, such as lung cancer or kidney cancer.

In some embodiments, provided herein is a method of treating lung cancer in a subject comprising administering to the subject an effective amount of an isolated IL-1β antibody or antigen binding fragment thereof as described herein. Administration may comprise, for example, systemic or local delivery.

In some embodiments, the subject has been diagnosed with lung cancer (e.g., non-small cell lung cancer). In some embodiments, the subject has been diagnosed with Stage 0 non-small cell lung cancer (NSCLC). In some embodiments, the subject has been diagnosed with Stage 1 NSCLC. In some embodiments, the subject has been diagnosed with Stage 2 NSCLC. In some embodiments, the subject has been diagnosed with Stage 2 NSCLC and has undergone surgery. In some embodiments, the subject has been diagnosed with Stage 3 NSCLC. In some embodiments, the subject has been diagnosed with Stage 3 NSCLC and undergone surgery. In some embodiments, the subject has been diagnosed with Stage 4 NSCLC.

In some embodiments, provided herein is a method of treating kidney cancer in a subject comprising administering to the subject an effective amount of an isolated IL-1β antibody or antigen binding fragment thereof as described herein.

In some embodiments, the subject has been diagnosed with kidney cancer e.g., renal cell carcinoma (RCC). In some embodiments, the subject has been diagnosed with Stage 1. In some embodiments, the subject has been diagnosed with Stage 2 RCC. In some embodiments, the subject has been diagnosed with Stage 3 RCC.

In some embodiments, antibodies provided herein are used for the interception of lung cancer. In some embodiments, the subject has been identified as being at risk of developing lung cancer. In some embodiments, provided herein is a method of reducing the risk of lung cancer in a subject comprising administering to the subject an effective amount of an isolated IL-1β antibody or antigen binding fragment thereof as described herein.

A subject at risk of developing lung cancer can be identified by various factors known in the art. In some embodiments, the subject has been determined to have one or more lung nodules, such as pre-cancerous lung nodules (e.g., as identified by computed tomographic imaging). In some embodiments, the one or more lung nodules are pre-cancerous. In some embodiments, the subject is between the ages of about 50 years old to about 80 years old and/or the subject has a history of smoking, for example, a 20 pack-year smoking history. In some embodiments, the subject has elevated levels of C-reactive protein (CRP).

According to an additional embodiment, a subject at risk of developing lung cancer is identified according to methods described in WO2021/146516 (“SYSTEM AND METHOD FOR PREDICTING THE RISK OF FUTURE LUNG CANCER”), which is incorporated by reference herein, and the at-risk subject is administered an effective amount of an isolated IL-1β antibody or antigen binding fragment thereof as described herein. For example, a method may comprise identifying a patient at risk of developing lung cancer by obtaining one or more images captured from the patient (e.g., CT scans); extracting features from the one or more obtained images (e.g., the extracted features comprising at least non-nodule specific features, wherein the non-nodule specific features comprise one or both of lung parenchyma features or body composition features); predicting one or more future risks of lung cancer for the subject by applying one or more trained risk prediction models to analyze the extracted features from the one or more obtained images; and, if the patient is identified as at-risk of developing lung cancer (e.g., at-risk of developing lung cancer within 1 year, 3 years, 5 years or 10 years), administering to the patient an effective amount of an isolated IL-1β antibody or antigen binding fragment thereof as described herein.

In some embodiments, antibodies provided herein are used for the prevention of lung cancer. The prevention may be complete, e.g., the total absence of an IL-1β-related condition or disorder. The prevention may also be partial, such that the likelihood of the occurrence of the IL-1β-related condition or metabolic disorder in a subject is less likely to occur than had the subject not received an antibody of the present disclosure.

Methods of administration and dosing is described in more detail in Section 5.7 below.

In another aspect, provided herein is the use of the antibody or antigen binding fragment thereof provided herein in the manufacture of a medicament for treating a disease or disorder in a subject.

In another aspect, provided herein is the use of a pharmaceutical composition provided herein in the manufacture of a medicament for treating a disease or disorder in a subject.

In another aspect, provided herein is the use of an antibody or antigen binding fragment thereof provided herein in the manufacture of a medicament, wherein the medicament is for use in a method for detecting the presence of an IL-1β in a biological sample, the method comprising contacting the biological sample with the antibody under conditions permissive for binding of the antibody to the IL-1β protein, and detecting whether a complex is formed between the antibody and the IL-1β protein.

In other aspects, the antibodies and fragments thereof of the present disclosure are useful for detecting the presence of an IL-1β in a biological sample. The term “detecting” as used herein encompasses quantitative or qualitative detection. In certain embodiments, a biological sample comprises bodily fluid, a cell, or a tissue. Diagnostic assays and methods are described in more detail in Section 5.9 below.

### 5.7 Methods of Administration and Dosing

In a specific embodiment, provided herein is a composition for use in the prevention and/or treatment of a disease or condition comprising an antibody or antigen binding fragment thereof provided herein. In one embodiment, provided herein is a composition for use in the prevention of a disease or condition, wherein the composition comprises an antibody or antigen binding fragment thereof provided herein. In one embodiment, provided herein is a composition for use in the treatment of a disease or condition, wherein the composition comprises an antibody or antigen binding fragment thereof provided herein. In some embodiments, the disease or condition is an IL-1β-mediated disease. In some embodiments, the disease or condition is an IL-1R1-mediated disease. In some embodiments, the disease or disorder is associated with IL-1β. In some embodiments, the disease or disorder is associated with IL-1β. In some embodiments, the disease or disorder is inflammatory disease or disorder. In some embodiments, the disease or disorder is cancer. In some embodiments, the cancer is lung cancer. In some embodiments, the lung cancer is non-small cell lung cancer. In some embodiments, the lung cancer is Stage 0 non-small cell lung cancer. In some embodiments, the lung cancer is Stage 1 non-small cell lung cancer. In some embodiments, the lung cancer is Stage 2 non-small cell lung cancer. In some embodiments, the lung cancer is Stage 3 non-small cell lung cancer. In some embodiments, the lung cancer is Stage 4 non-small cell lung cancer. In some embodiments, the cancer is kidney cancer. In some embodiments, the kidney cancer is renal cell cancer. In some embodiments, the renal cell cancer is Stage 1 renal cell cancer. In some embodiments, the renal cell cancer is Stage 2 renal cell cancer. In some embodiments, the renal cell cancer is Stage 3 renal cell cancer. In some embodiments, the subject is a subject in need thereof. In some embodiments, the subject has the disease or condition. In other embodiments, the subject is at risk of having the disease or condition. In some embodiments, the administer action results in the prevention, management, treatment or amelioration of the disease or condition.

In one embodiment, provided herein is a composition for use in the prevention and/or treatment of a symptom of a disease or condition, wherein the composition comprises an antibody or antigen binding fragment thereof provided herein. In one embodiment, provided herein is a composition for use in the prevention of a symptom of a disease or condition, wherein the composition comprises an antibody or antigen binding fragment thereof provided herein. In one embodiment, provided herein is a composition for use in the treatment of a symptom of a disease or condition, wherein the composition comprises an antibody or antigen binding fragment thereof provided herein. In some embodiments, the disease or condition is an IL-1β-mediated disease. In some embodiments, the disease or condition is an IL-1R1-mediated disease. In some embodiments, the disease or disorder is associated with IL-1β. In some embodiments, the disease or disorder is associated with IL-1β. In some embodiments, the disease or disorder is inflammatory disease or disorder. In some embodiments, the disease or disorder is cancer. In some embodiments, the cancer is lung cancer. In some embodiments, the lung cancer is non-small cell lung cancer. In some embodiments, the lung cancer is Stage 0 non-small cell lung cancer. In some embodiments, the lung cancer is Stage 1 non-small cell lung cancer. In some embodiments, the lung cancer is Stage 2 non-small cell lung cancer. In some embodiments, the lung cancer is Stage 3 non-small cell lung cancer. In some embodiments, the lung cancer is Stage 4 non-small cell lung cancer. In some embodiments, the cancer is kidney cancer. In some embodiments, the kidney cancer is renal cell cancer. In some embodiments, the renal cell cancer is Stage 1 renal cell cancer. In some embodiments, the renal cell cancer is Stage 2 renal cell cancer. In some embodiments, the renal cell cancer is Stage 3 renal cell cancer. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of an antibody or antigen binding fragment thereof provided herein. In one embodiment, provided herein is a method of preventing a disease or condition in a subject, comprising administering an effective amount of an antibody or antigen binding fragment thereof provided herein. In one embodiment, provided herein is a method of treating a disease or condition in a subject, comprising administering an effective amount of an antibody or antigen binding fragment thereof provided herein. In some embodiments, the disease or condition is an IL-1β-mediated disease. In some embodiments, the disease or condition is an IL-1R1-mediated disease. In some embodiments, the disease or disorder is associated with IL-1β. In some embodiments, the disease or disorder is associated with IL-1β. In some embodiments, the disease or disorder is inflammatory disease or disorder. In some embodiments, the disease or disorder is cancer. In some embodiments, the cancer is lung cancer. In some embodiments, the lung cancer is non-small cell lung cancer. In some embodiments, the lung cancer is Stage 0 non-small cell lung cancer. In some embodiments, the lung cancer is Stage 1 non-small cell lung cancer. In some embodiments, the lung cancer is Stage 2 non-small cell lung cancer. In some embodiments, the lung cancer is Stage 3 non-small cell lung cancer. In some embodiments, the lung cancer is Stage 4 non-small cell lung cancer. In some embodiments, the cancer is kidney cancer. In some embodiments, the kidney cancer is renal cell cancer. In some embodiments, the renal cell cancer is Stage 1 renal cell cancer. In some embodiments, the renal cell cancer is Stage 2 renal cell cancer. In some embodiments, the renal cell cancer is Stage 3 renal cell cancer. In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody or antigen binding fragment thereof provided herein. In one embodiment, provided herein is a method of preventing a symptom of a disease or condition in a subject, comprising administering an effective amount of an antibody or antigen binding fragment thereof provided herein. In one embodiment, provided herein is a method of treating a symptom of a disease or condition in a subject, comprising administering an effective amount of an antibody or antigen binding fragment thereof provided herein. In some embodiments, the disease or condition is an IL-1β-mediated disease. In some embodiments, the disease or condition is an IL-1R1-mediated disease. In some embodiments, the disease or disorder is associated with IL-1β. In some embodiments, the disease or disorder is associated with IL-1β. In some embodiments, the disease or disorder is inflammatory disease or disorder. In some embodiments, the disease or disorder is cancer. In some embodiments, the cancer is lung cancer. In some embodiments, the lung cancer is non-small cell lung cancer. In some embodiments, the lung cancer is Stage 0 non-small cell lung cancer. In some embodiments, the lung cancer is Stage 1 non-small cell lung cancer. In some embodiments, the lung cancer is Stage 2 non-small cell lung cancer. In some embodiments, the lung cancer is Stage 3 non-small cell lung cancer. In some embodiments, the lung cancer is Stage 4 non-small cell lung cancer. In some embodiments, the cancer is kidney cancer. In some embodiments, the kidney cancer is renal cell cancer. In some embodiments, the renal cell cancer is Stage 1 renal cell cancer. In some embodiments, the renal cell cancer is Stage 2 renal cell cancer. In some embodiments, the renal cell cancer is Stage 3 renal cell cancer. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

Also provided herein are methods of preventing and/or treating a disease or condition by administrating to a subject of an effective amount of an antibody or antigen binding fragment thereof provided herein, or pharmaceutical composition comprising an antibody or antigen binding fragment thereof provided herein. In one aspect, the antibody or antigen binding fragment thereof is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus macaque monkey, or a human). In a one embodiment, the subject is a human. In another embodiment, the subject is a human with a disease or condition.

Various delivery systems are known and can be used to administer a prophylactic or therapeutic agent (e.g., an antibody or antigen binding fragment thereof provided herein). Methods of administering a prophylactic or therapeutic agent (e.g., an antibody or antigen binding fragment thereof provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody or antigen binding fragment thereof provided herein), or a pharmaceutical composition is administered intranasally, intramuscularly, intravenously, or subcutaneously.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant

In a specific embodiment, a composition provided herein comprises one, two or more antibodies or antigen binding fragments thereof provided herein. In another embodiment, a composition provided herein comprises one, two or more antibodies or antigen binding fragments thereof provided herein and a prophylactic or therapeutic agent other than an antibody or antigen binding fragment thereof provided herein. In one embodiment, the agents are known to be useful for or have been or are currently used for the prevention, management, treatment and/or amelioration of a disease or condition. In addition to prophylactic or therapeutic agents, the compositions provided herein may also comprise one or more excipients.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody or antigen binding fragment thereof provided herein or other prophylactic or therapeutic agent), and one or more pharmaceutically acceptable excipients. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin. Water is an exemplary excipient when the pharmaceutical composition is administered intravenously. Saline solutions and aqueous dextrose and glycerol solutions can also be employed as liquid excipients, particularly for injectable solutions. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Non-limiting examples of suitable pharmaceutical excipients are described in Remington's Pharmaceutical Sciences (1990) Mack Publishing Co., Easton, Pa. Such compositions will contain a prophylactically or therapeutically effective amount of the antibody or antigen binding fragment thereof provided herein, such as in purified form, together with a suitable amount of excipient(s) so as to provide the form for proper administration to the patient. The formulation should suit the mode of administration.

In an embodiment, the composition is formulated in accordance with procedures as a pharmaceutical composition adapted for intravenous administration to human beings.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody or antigen binding fragment thereof provided herein can be packaged in a hermetically sealed container such as an ampoule or sachette indicating the quantity of antibody. In one embodiment, the antibody or antigen binding fragment thereof is supplied as a dry sterilized lyophilized powder or water free concentrate in a hermetically sealed container and can be reconstituted, e.g., with water or saline to the appropriate concentration for administration to a subject.

The compositions provided herein can be formulated as neutral or salt forms. Pharmaceutically acceptable salts include those formed with anions and those formed with cations.

The amount of a prophylactic or therapeutic agent (e.g., an antibody or antigen binding fragment thereof provided herein), or a composition provided herein that will be effective in the prevention and/or treatment of a disease or condition can be determined by clinical techniques known in the art. The precise dose to be employed in the formulation will also depend on the route of administration, and the seriousness of a disease or condition, and should be decided according to the judgment of the practitioner and each patient's circumstances.

In certain embodiments, the route of administration for a dose of an antibody or antigen binding fragment thereof provided herein to a patient is intranasal, intramuscular, intravenous, subcutaneous, or a combination thereof, but other routes described herein are also acceptable. Each dose may or may not be administered by an identical route of administration. In some embodiments, an antibody or antigen binding fragment thereof provided herein may be administered via multiple routes of administration simultaneously or subsequently to other doses of the same or a different antibody or antigen binding fragment thereof provided herein.

In certain embodiments, the antibody or antigen binding fragment thereof provided herein are administered prophylactically or therapeutically to a subject. The antibody or antigen binding fragment thereof provided herein can be prophylactically or therapeutically administered to a subject so as to prevent, lessen or ameliorate a disease or symptom thereof.

### 5.8 Gene Therapy

In a specific embodiment, nucleic acids comprising sequences encoding antibodies or functional derivatives thereof, are administered to a subject for use in a method provided herein, for example, to prevent, manage, treat and/or ameliorate an IL-1β-mediated disease, disorder or condition, by way of gene therapy. Such therapy encompasses that performed by the administration to a subject of an expressed or expressible nucleic acid. In an embodiment, the nucleic acids produce their encoded antibody, and the antibody mediates a prophylactic or therapeutic effect.

Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, N Y (1993); and Kriegler, Gene Transfer and Expression, A Laboratory Manual, Stockton Press, NY (1990).

In a specific embodiment, a composition comprises nucleic acids encoding an antibody provided herein, the nucleic acids being part of an expression vector that expresses the antibody or chimeric proteins or heavy or light chains thereof in a suitable host. In particular, such nucleic acids have promoters, such as heterologous promoters, operably linked to the antibody coding region, the promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438).

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences (e.g. DNA or mRNA sequences) are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA or mRNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an anti-IL-1β antibody as provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for gene therapy is autologous to the subject.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.9. Diagnostic Assays and Methods

Labeled antibodies and derivatives and analogs thereof, which immunospecifically bind to an IL-1β antigen can be used for diagnostic purposes to detect, diagnose, or monitor an IL-1β-mediated disease. Thus, provided herein are methods for the detection of an IL-1β-mediated disease comprising: (a) assaying the expression of an IL-1β antigen in cells or a tissue sample of a subject using one or more antibodies provided herein that immunospecifically bind to the IL-1β antigen; and (b) comparing the level of the IL-1β antigen with a control level, e.g., levels in normal tissue samples (e.g., from a patient not having an IL-1β-mediated disease, or from the same patient before disease onset), whereby an increase in the assayed level of IL-1β antigen compared to the control level of the IL-1β antigen is indicative of an IL-1β-mediated disease.

Also provided herein is a diagnostic assay for diagnosing an IL-1β-mediated disease comprising: (a) assaying for the level of an IL-1β antigen in cells or a tissue sample of an individual using one or more antibodies provided herein that immunospecifically bind to an IL-1β antigen; and (b) comparing the level of the IL-1β antigen with a control level, e.g., levels in normal tissue samples, whereby an increase in the assayed IL-1β antigen level compared to the control level of the IL-1β antigen is indicative of an IL-1β-mediated disease. In certain embodiments, provided herein is a method of treating an IL-1β-mediated disease in a subject, comprising: (a) assaying for the level of an IL-1β antigen in cells or a tissue sample of the subject using one or more antibodies provided herein that immunospecifically bind to an IL-1β antigen; and (b) comparing the level of the IL-1β antigen with a control level, e.g., levels in normal tissue samples, whereby an increase in the assayed IL-1β antigen level compared to the control level of the IL-1β antigen is indicative of an IL-1β-mediated disease. In some embodiments, the method further comprises (c) administering an effective amount of an antibody provided herein to the subject identified as having the IL-1β-mediated disease. A more definitive diagnosis of an IL-1β-mediated disease may allow health professionals to employ preventative measures or aggressive treatment earlier thereby preventing the development or further progression of the IL-1β-mediated disease.

Antibodies provided herein can be used to assay IL-1β antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (1251, 1211), carbon (14C), sulfur (35S), tritium (3H), indium (1211n), and technetium (99Tc); luminescent labels, such as luminol; and fluorescent labels, such as fluorescein and rhodamine, and biotin.

One aspect provided herein is the detection and diagnosis of an IL-1β-mediated disease in a human. In one embodiment, diagnosis comprises: a) administering (for example, parenterally, subcutaneously, or intraperitoneally) to a subject an effective amount of a labeled antibody that immunospecifically binds to an IL-1β antigen; b) waiting for a time interval following the administering for permitting the labeled antibody to concentrate at sites in the subject where the IL-1β antigen is expressed (and for unbound labeled molecule to be cleared to background level); c) determining background level; and d) detecting the labeled antibody in the subject, such that detection of labeled antibody above the background level indicates that the subject has an IL-1β-mediated disease. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of an IL-1β-mediated disease is carried out by repeating the method for diagnosing an IL-1β-mediated disease, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MM), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.10 Kits

Also provided herein are kits comprising an antibody (e.g., an anti-IL-1β antibody) provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to IL-1β antigen. In specific embodiments, provided herein are panels of antibodies having different association rate constants different dissociation rate constants, different affinities for IL-1β antigen, and/or different specificities for an IL-1β antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. Embodiments

This invention provides the following non-limiting embodiments.

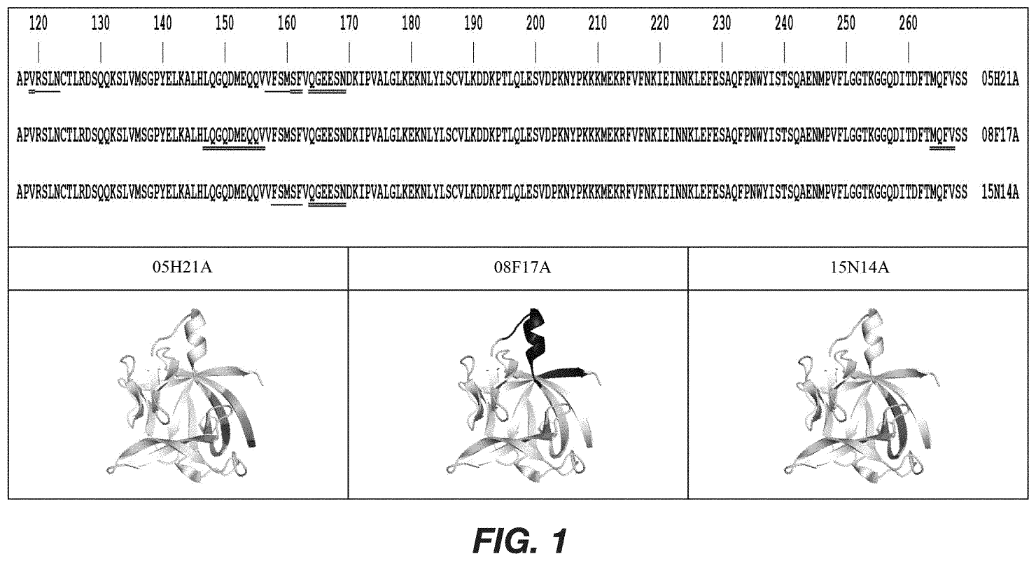

In one set of embodiments, provided are:
1. An antibody that binds IL-1β comprising:
   (1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 8;
   (2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 9; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 10; or
   (3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 11; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 12.
2. The antibody of embodiment 1, (i) wherein the VH CDR1, VH CDR2, VH CDR3, VL CDR1, VL CDR2, and VL CDR3 amino acid sequences are according to the Kabat numbering system; (ii) wherein the VH CDR1, VH CDR2, VH CDR3, VL CDR1, VL CDR2, and VL CDR3 amino acid sequences are according to the Chothia numbering system; (iii) wherein the VH CDR1, VH CDR2, VH CDR3, VL CDR1, VL CDR2, and VL CDR3 amino acid sequences are according to the AbM numbering system; (iv) wherein the VH CDR1, VH CDR2, VH CDR3, VL CDR1, VL CDR2, and VL CDR3 amino acid sequences are according to the Contact numbering system; and/or (v) wherein the VH CDR1, VH CDR2, VH CDR3, VL CDR1, VL CDR2, and VL CDR3 amino acid sequences are according to the IMGT numbering system.
3. An antibody that binds IL-1β comprising:
   (1) (i) a VH comprising a VH CDR1 having an amino acid sequence selected from SEQ ID NO: 13, SEQ ID NO: 31, SEQ ID NO: 49, SEQ ID NO: 67, and SEQ ID NO: 85; a VH CDR2 having an amino acid sequence selected from SEQ ID NO: 14, SEQ ID NO: 32, SEQ ID NO: 50, SEQ ID NO: 68, and SEQ ID NO: 86; a VH CDR3 having an amino acid sequence selected from SEQ ID NO: 15, SEQ ID NO: 33, SEQ ID NO: 51, SEQ ID NO: 69, and SEQ ID NO: 87; and
      (ii) a VL comprising a VL CDR1 having an amino acid sequence selected from SEQ ID NO: 16, SEQ ID NO: 34, SEQ ID NO: 52, SEQ ID NO: 70, and SEQ ID NO: 88; a VL CDR2 having an amino acid sequence selected from SEQ ID NO: 17, SEQ ID NO: 35, SEQ ID NO: 53, SEQ ID NO: 71, and SEQ ID NO: 89; a VL CDR3 having an amino acid sequence selected from SEQ ID NO: 18, SEQ ID NO: 36, SEQ ID NO: 54, SEQ ID NO: 72, and SEQ ID NO: 90;
   (2) (i) a VH comprising a VH CDR1 having an amino acid sequence selected from SEQ ID NO: 19, SEQ ID NO: 37, SEQ ID NO: 55, SEQ ID NO: 73, and SEQ ID NO: 91; a VH CDR2 having an amino acid sequence selected from SEQ ID NO: 20, SEQ ID NO: 38, SEQ ID NO: 56, SEQ ID NO: 74, and SEQ ID NO: 92; a VH CDR3 having an amino acid sequence selected from SEQ ID NO: 21, SEQ ID NO: 39, SEQ ID NO: 57, SEQ ID NO: 75, and SEQ ID NO: 93; and
      (ii) a VL comprising a VL CDR1 having an amino acid sequence selected from SEQ ID NO: 22, SEQ ID NO: 40, SEQ ID NO: 58, SEQ ID NO: 76, and SEQ ID NO: 94; a VL CDR2 having an amino acid sequence selected from SEQ ID NO: 23, SEQ ID NO: 41, SEQ ID NO: 59, SEQ ID NO: 77, and SEQ ID NO: 95; a VL CDR3 having an amino acid sequence selected from SEQ ID NO: 24, SEQ ID NO: 42, SEQ ID NO: 60, SEQ ID NO: 78, and SEQ ID NO: 96; or
   (3) (i) a VH comprising a VH CDR1 having an amino acid sequence selected from SEQ ID NO: 25, SEQ ID NO: 43, SEQ ID NO: 61, SEQ ID NO: 79, and SEQ ID NO: 97; a VH CDR2 having an amino acid sequence selected from SEQ ID NO: 26, SEQ ID NO: 44, SEQ ID NO: 62, SEQ ID NO: 80, and SEQ ID NO: 98; a VH CDR3 having an amino acid sequence selected from SEQ ID NO: 27, SEQ ID NO: 45, SEQ ID NO: 63, SEQ ID NO: 81, and SEQ ID NO: 99; and
      (ii) a VL comprising a VL CDR1 having an amino acid sequence selected from SEQ ID NO: 28, SEQ ID NO: 46, SEQ ID NO: 64, SEQ ID NO: 82, and SEQ ID NO: 100; a VL CDR2 having an amino acid sequence selected from SEQ ID NO: 29, SEQ ID NO: 47, SEQ ID NO: 65, SEQ ID NO: 83, and SEQ ID NO: 101; a VL CDR3 having an amino acid sequence selected from SEQ ID NO: 30, SEQ ID NO: 48, SEQ ID NO: 66, SEQ ID NO: 84, and SEQ ID NO: 102.
4. An antibody that binds IL-1β comprising:
   (1) (i) a VH comprising a VH CDR1 having an amino acid sequence of SEQ ID NO: 13; a VH CDR2 having an amino acid sequence of SEQ ID NO: 14; a VH CDR3 having an amino acid sequence of SEQ ID NO: 15; and
      (ii) a VL comprising a VL CDR1 having an amino acid sequence of SEQ ID NO: 16; a VL CDR2 having an amino acid sequence selected of SEQ ID NO: 17; a VL CDR3 having an amino acid sequence of SEQ ID NO: 18;
   (2) (i) a VH comprising a VH CDR1 having an amino acid sequence of SEQ ID NO: 19; a VH CDR2 having an amino acid sequence of SEQ ID NO: 20; a VH CDR3 having an amino acid sequence of SEQ ID NO: 21; and
      (ii) a VL comprising a VL CDR1 having an amino acid sequence of SEQ ID NO: 22; a VL CDR2 having an amino acid sequence selected of SEQ ID NO: 23; a VL CDR3 having an amino acid sequence of SEQ ID NO: 24;
   (3) (i) a VH comprising a VH CDR1 having an amino acid sequence of SEQ ID NO: 25; a VH CDR2 having an amino acid sequence of SEQ ID NO: 26; a VH CDR3 having an amino acid sequence of SEQ ID NO: 27; and
      (ii) a VL comprising a VL CDR1 having an amino acid sequence of SEQ ID NO: 28; a VL CDR2 having an amino acid sequence selected of SEQ ID NO: 29; a VL CDR3 having an amino acid sequence of SEQ ID NO: 30;
   (4) (i) a VH comprising a VH CDR1 having an amino acid sequence of SEQ ID NO: 31; a VH CDR2 having an amino acid sequence of SEQ ID NO: 32; a VH CDR3 having an amino acid sequence of SEQ ID NO: 33; and
      (ii) a VL comprising a VL CDR1 having an amino acid sequence of SEQ ID NO: 34; a VL CDR2 having an amino acid sequence selected of SEQ ID NO: 35; a VL CDR3 having an amino acid sequence of SEQ ID NO: 36;
   (5) (i) a VH comprising a VH CDR1 having an amino acid sequence of SEQ ID NO: 37; a VH CDR2 having an amino acid sequence of SEQ ID NO: 38; a VH CDR3 having an amino acid sequence of SEQ ID NO: 39; and
      (ii) a VL comprising a VL CDR1 having an amino acid sequence of SEQ ID NO: 40; a VL CDR2 having an amino acid sequence selected of SEQ ID NO: 41; a VL CDR3 having an amino acid sequence of SEQ ID NO: 42;
   (6) (i) a VH comprising a VH CDR1 having an amino acid sequence of SEQ ID NO: 43; a VH CDR2 having an amino acid sequence of SEQ ID NO: 44; a VH CDR3 having an amino acid sequence of SEQ ID NO: 45; and
      (ii) a VL comprising a VL CDR1 having an amino acid sequence of SEQ ID NO: 46; a VL CDR2 having an amino acid sequence selected of SEQ ID NO: 47; a VL CDR3 having an amino acid sequence of SEQ ID NO: 48;
   (7) (i) a VH comprising a VH CDR1 having an amino acid sequence of SEQ ID NO: 49; a VH CDR2 having an amino acid sequence of SEQ ID NO: 50; a VH CDR3 having an amino acid sequence of SEQ ID NO: 51; and
      (ii) a VL comprising a VL CDR1 having an amino acid sequence of SEQ ID NO: 52; a VL CDR2 having an amino acid sequence selected of SEQ ID NO: 53; a VL CDR3 having an amino acid sequence of SEQ ID NO: 54;
   (8) (i) a VH comprising a VH CDR1 having an amino acid sequence of SEQ ID NO: 55; a VH CDR2 having an amino acid sequence of SEQ ID NO: 56; a VH CDR3 having an amino acid sequence of SEQ ID NO: 57; and
      (ii) a VL comprising a VL CDR1 having an amino acid sequence of SEQ ID NO: 58; a VL CDR2 having an amino acid sequence selected of SEQ ID NO: 59; a VL CDR3 having an amino acid sequence of SEQ ID NO: 60;
   (9) (i) a VH comprising a VH CDR1 having an amino acid sequence of SEQ ID NO: 61; a VH CDR2 having an amino acid sequence of SEQ ID NO: 62; a VH CDR3 having an amino acid sequence of SEQ ID NO: 63; and
      (ii) a VL comprising a VL CDR1 having an amino acid sequence of SEQ ID NO: 64; a VL CDR2 having an amino acid sequence selected of SEQ ID NO: 65; a VL CDR3 having an amino acid sequence of SEQ ID NO: 66;
   (10) (i) a VH comprising a VH CDR1 having an amino acid sequence of SEQ ID NO: 67; a VH CDR2 having an amino acid sequence of SEQ ID NO: 68; a VH CDR3 having an amino acid sequence of SEQ ID NO: 69; and
      (ii) a VL comprising a VL CDR1 having an amino acid sequence of SEQ ID NO: 70; a VL CDR2 having an amino acid sequence selected of SEQ ID NO: 71; a VL CDR3 having an amino acid sequence of SEQ ID NO: 72;
   (11) (i) a VH comprising a VH CDR1 having an amino acid sequence of SEQ ID NO: 73; a VH CDR2 having an amino acid sequence of SEQ ID NO: 74; a VH CDR3 having an amino acid sequence of SEQ ID NO: 75; and
      (ii) a VL comprising a VL CDR1 having an amino acid sequence of SEQ ID NO: 76; a VL CDR2 having an amino acid sequence selected of SEQ ID NO: 77; a VL CDR3 having an amino acid sequence of SEQ ID NO: 78;
   (12) (i) a VH comprising a VH CDR1 having an amino acid sequence of SEQ ID NO: 79; a VH CDR2 having an amino acid sequence of SEQ ID NO: 80; a VH CDR3 having an amino acid sequence of SEQ ID NO: 81; and
      (ii) a VL comprising a VL CDR1 having an amino acid sequence of SEQ ID NO: 82; a VL CDR2 having an amino acid sequence selected of SEQ ID NO: 83; a VL CDR3 having an amino acid sequence of SEQ ID NO: 84;
   (13) (i) a VH comprising a VH CDR1 having an amino acid sequence of SEQ ID NO: 85; a VH CDR2 having an amino acid sequence of SEQ ID NO: 86; a VH CDR3 having an amino acid sequence of SEQ ID NO: 87; and
      (ii) a VL comprising a VL CDR1 having an amino acid sequence of SEQ ID NO: 88; a VL CDR2 having an amino acid sequence selected of SEQ ID NO: 89; a VL CDR3 having an amino acid sequence of SEQ ID NO: 90;
   (14) (i) a VH comprising a VH CDR1 having an amino acid sequence of SEQ ID NO: 91; a VH CDR2 having an amino acid sequence of SEQ ID NO: 92; a VH CDR3 having an amino acid sequence of SEQ ID NO: 93; and
      (ii) a VL comprising a VL CDR1 having an amino acid sequence of SEQ ID NO: 94; a VL CDR2 having an amino acid sequence selected of SEQ ID NO: 95; a VL CDR3 having an amino acid sequence of SEQ ID NO: 96; or
   (15) (i) a VH comprising a VH CDR1 having an amino acid sequence of SEQ ID NO: 97; a VH CDR2 having an amino acid sequence of SEQ ID NO: 98; a VH CDR3 having an amino acid sequence of SEQ ID NO: 99; and
      (ii) a VL comprising a VL CDR1 having an amino acid sequence of SEQ ID NO: 100; a VL CDR2 having an amino acid sequence selected of SEQ ID NO: 101; a VL CDR3 having an amino acid sequence of SEQ ID NO: 102.
5. The antibody of any one of embodiments 1 to 4, wherein the antibody further comprises one or more framework regions as set forth in SEQ ID NO: 7, SEQ ID NO: 8, SEQ ID NO: 9, SEQ ID NO: 10, SEQ ID NO: 11, and/or SEQ ID NO: 12.
6. The antibody of any one of embodiments 1 to 5, wherein:
   (i) the antibody comprises a VH having an amino acid sequence of SEQ ID NO: 7, and a VL having an amino acid sequence of SEQ ID NO: 8;
   (ii) the antibody comprises a VH having an amino acid sequence of SEQ ID NO: 9, and a VL having an amino acid sequence of SEQ ID NO: 10; or
   (iii) the antibody comprises a VH having an amino acid sequence of SEQ ID NO: 11, and a VL having an amino acid sequence of SEQ ID NO: 12.
7 The antibody of any one of embodiments 1 to 6, wherein the antibody is a humanized antibody.
8. The antibody of any one of embodiments 1 to 7, wherein the antibody is an IgG antibody.
9. The antibody of embodiment 8, wherein the IgG antibody is an IgG1, IgG2, IgG3, or IgG4 antibody.
10. The antibody of any one of embodiments 1 to 9, wherein the antibody comprises a kappa light chain.
11. The antibody of any one of embodiments 1 to 9, wherein the antibody comprises a lambda light chain.
12. The antibody of any one of embodiments 1 to 11, wherein the antibody comprises a mutant Fc region.
13. The antibody of embodiment 12, wherein the mutant Fc region comprises M252Y/S254T/T256E (YTE) mutations.
14. The antibody of any one of embodiments 1 to 13, wherein the antibody is a monoclonal antibody.
15. The antibody of any one of embodiments 1 to 14, wherein the antibody binds an IL-1β antigen.
16. The antibody of any one of embodiments 1 to 14, wherein the antibody binds an IL-1β epitope.
17. The antibody of any one of embodiments 1 to 14, wherein the antibody specifically binds to IL-1β.
18. The antibody of any one of embodiments 1 to 17, wherein the VH CDR1, VH CDR2, VH CDR3, VL CDR1, VL CDR2 and VL CDR3 form a binding site for an antigen of the IL-1β.
19. The antibody of any one of embodiments 1 to 15, wherein the VH CDR1, VH CDR2, VH CDR3, VL CDR1, VL CDR2 and VL CDR3 form a binding site for an epitope of the IL-1β.
20. The antibody of any one of embodiments 1 to 19, wherein the antibody is multispeicfic.
21. The antibody of embodiment 20, wherein the antibody is capable of binding at least two antigens.
22. The antibody of embodiment 20, wherein the antibody is capable of binding at least three antigens.
23. The antibody of embodiment 20, wherein the antibody is capable of binding at least four antigens.
24. The antibody of embodiment 20, wherein the antibody is capable of binding at least five antigens.
25. A binding molecule comprising the antibody of any one of embodiments 1 to 24, wherein the antibody is genetically fused or chemically conjugated to an agent.
26. A nucleic acid encoding the antibody of any one of embodiments 1 to 24.
27. A vector comprising the nucleic acid of embodiment 26.
28. A host cell comprising the vector of embodiment 27.
29. A kit comprising the vector of embodiment 27 and packaging for the same.
30. A kit comprising the antibody of any one of embodiments 1 to 24 and packaging for the same.
31. A pharmaceutical composition comprising the antibody of any one of embodiments 1 to 24, and one or more pharmaceutically acceptable excipients.
32. A method of producing the pharmaceutical composition of embodiment 31, comprising combining the antibody with one or more pharmaceutically acceptable excipients to obtain the pharmaceutical composition.
33. A method of inhibiting IL-1β or IL-1β mediated signaling in a cell, comprising the contacting the cell with the antibody of any one of embodiments 1 to 24.
34. A method of inhibiting IL-1β induced production of IL-6, ENA-78 (CXCL5) and/or G-CSF in a cell, comprising the contacting the cell with the antibody of any one of embodiments 1 to 24.
35. A method of decreasing the production of IL-6, ENA-78 (CXCL5) and/or G-CSF in a cell, comprising the contacting the cell with the antibody of any one of embodiments 1 to 24.
36. A method inhibiting growth or proliferation of IL-1β expressing cells, comprising contacting the cells with the antibody of any one of embodiments 1 to 24.
37. The method of any one of embodiments 33 to 36, wherein the cell or the cells are in a subject having a disease or disorder.
38. A method of inhibiting IL-1β in a subject, comprising administering to the subject the antibody of any one of embodiments 1 to 24.
39. A method for treating a disease or disorder in a subject, comprising administering to the subject the antibody of any one of embodiments 1 to 24.
40. The method of embodiment 37 or 39, wherein the disease or disorder is an IL-1β associated disease or disorder.
41. The method of embodiment 40, wherein the IL-1β associated disease or disorder is an inflammatory disease or disorder.
42. The method of embodiment 40, wherein the IL-1β associated disease or disorder is cancer.
43. The method of embodiment 42, wherein the cancer is lung cancer.
44. The method of embodiment 43, wherein the lung cancer is non-small cell lung cancer, wherein optionally the non-small cell lung cancer has reached stage 0, stage 1, stage 2, stage 3, or stage 4.
45. The method of embodiment 42, wherein the cancer is kidney cancer
46. The method of embodiment 45, wherein the kidney cancer is renal cell cancer.
47. The method of embodiment 46, wherein the renal cell cancer has reached stage 1, stage 2, or stage 3.
48. An isolated protein comprising an antigen binding domain that binds hIL-1β, wherein said antigen binding domain binds to an epitope on hIL-1β having a sequence selected from the epitopes identified in FIG. 1.
49. The isolated protein of embodiment 48, wherein the isolated protein binds to the same epitope(s) as of 05H21A.
50. The isolated protein of embodiment 48, wherein the isolated protein binds to the same epitope(s) as of 15N14A.
51. The isolated protein of embodiment 48, wherein the isolated protein binds to the same epitope(s) as of 08F17A.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### 7. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 7.1 Example 1: IL-1β mAb Molecule Design, Sequence and Structure

High-affinity and potent anti-IL-1β antibody molecules were generated which are capable of neutralizing the IL-1β signaling pathway. A DNA immunization campaign encoding for a variant of human IL-1β with reduced bioactivity (D145K) was carried out in the Alivamab transgenic mouse platform. Three leads (clones: 05H21A, 15N14A, and 08F17A) described herein were Fc engineered to include a YTE mutation (M252Y/S254T/T256E) to enhance the antibody half-life.

The amino acid sequences for the HC and LC of the lead candidates are shown below in Table 3. The VL and VH amino acid sequences of the lead candidates are shown in Table 4. The 3 complementarity-determining regions (CDRs) in each chain of the lead candidates are shown in Table 5 to Table 9.

TABLE 3
HC and LC AA Sequences of the Lead anti-IL-1β mAbs

| Name | HC AA Seq | LC AA Seq |
|---|---|---|
| 05H21A | QVTLRESGPALVKPTQTLTLTCT | QSVLTQPPSVSEAPRQRVTISCS |
| Heavy Chain | FSGFSLSTSGMWVSWIRQPPGK | GSSSNIGDNAVNWYQQLPGKAP |
| isotype: IgG1- | ALEWLALIDWGDDKYYTTSLK | KLLIYNDDLLSSGVSDRFSGSKS |
| YTE | TRLTISKDTSKNQVVLTMTNMD | GTSASLAISGLQSEDEADYYCA |
| Light Chain | PVDTATYYCARMREGSRAFDI | AWDDSLNGPVFGGGTKLTVLG |
| Isotype: | WGQGTVVTVSSASTKGPSVFPL | QPKAAPSVTLFPPSSEELQANKA |
| Lambda | APSSKSTSGGTAALGCLVKDYF | TLVCLISDFYPGAVTVAWKADS |
| | PEPVTVSWNSGALTSGVHTFPA | SPVKAGVETTTPSKQSNNKYAA |
| | VLQSSGLYSLSSVVTVPSSSLGT | SSYLSLTPEQWKSHRSYSCQVT |
| | QTYICNVNHKPSNTKVDKKVEP | HEGSTVEKTVAPTECS |
| | KSCDKTHTCPPCPAPELLGGPSV | (SEQ ID NO: 2) |
| | FLFPPKPKDTLYITREPEVTCVV | |
| | VDVSHEDPEVKFNWYVDGVEV | |
| | HNAKTKPREEQYNSTYRVVSVL | |
| | TVLHQDWLNGKEYKCKVSNKA | |
| | LPAPIEKTISKAKGQPREPQVYT | |
| | LPPSREEMTKNQVSLTCLVKGF | |
| | YPSDIAVEWESNGQPENNYKTT | |
| | PPVLDSDGSFFLYSKLTVDKSR | |
| | WQQGNVFSCSVMHEALHNHYT | |
| | QKSLSLSPGK (SEQ ID NO: 1) | |
| 15N14A | QVQLQESGPGLVKPSETLSLTCT | QAVLTQPSSLSASPGASASLTCT |
| Heavy Chain | VSGGSISSYYWTWIRQPAGKGL | LRSGINVGTYRIYWYQQKPGSP |
| isotype: IgG1- | EWIGRIDSSGSSKYNPTLKSRVT | PQYLLSYKSDSDKQQGSGVPSR |
| YTE | MSVDTSKNQFSLKLSSVTAADT | FSGSKDASANVGILLISGLQSED |
| Light Chain | AVYYCARGDSGYDWAFDYWG | EADYYCMIWHSSAWVFGGGTK |
| Isotype: | QGTLVTVSSASTKGPSVFPLAPS | LTVLGQPKAAPSVTLFPPSSEEL |
| Lambda | SKSTSGGTAALGCLVKDYFPEP | QANKATLVCLISDFYPGAVTVA |
| | VTVSWNSGALTSGVHTFPAVLQ | WKADSSPVKAGVETTTPSKQSN |
| | SSGLYSLSSVVTVPSSSLGTQTYI | NKYAASSYLSLTPEQWKSHRSY |
| | CNVNHKPSNTKVDKKVEPKSC | SCQVTHEGSTVEKTVAPTECS |
| | DKTHTCPPCPAPELLGGPSVFLF | (SEQ ID NO: 4) |
| | PPKPKDTLYITREPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APIEKTISKAKGQPREPQVYTLP | |
| | PSREEMTKNQVSLTCLVKGFYP | |
| | SDIAVEWESNGQPENNYKTTPP | |
| | VLDSDGSFFLYSKLTVDKSRWQ | |
| | QGNVFSCSVMHEALHNHYTQK | |
| | SLSLSPGK (SEQ ID NO: 3) | |
| 08F17A | EVQLVESGGGLVTPGGSLRLSC | DIQMTQSPSSLSASVGDRVTITC |
| Heavy Chain | AASGFTFSGYSMNWVRQAPGK | RASQGISYYLAWYQQKPGKVP |
| isotype: IgG1- | GLEWVSSISSSSGYIYYADSVKG | KLLISAEFTLQSGVPSRFSGSGS |
| YTE | RFTISRDNAKNSLYLQMNSLRA | GTDFTLTISSLQPEDVATYYCQK |
| Light Chain | EDTAVYYCAREYWGSGFDYW | YNTAPRTFGQGTKVEIKRTVAA |
| Isotype: | GQGTLVTVSSASTKGPSVFPLAP | PSVFIFPPSDEQLKSGTASVVCL |
| Kappa | SSKSTSGGTAALGCLVKDYFPE | LNNFYPREAKVQWKVDNALQS |
| | PVTVSWNSGALTSGVHTFPAVL | GNSQESVTEQDSKDSTYSLSSTL |
| | QSSGLYSLSSVVTVPSSSLGTQT | TLSKADYEKHKVYACEVTHQG |
| | YICNVNHKPSNTKVDKKVEPKS | LSSPVTKSFNRGEC |
| | CDKTHTCPPCPAPELLGGPSVFL | (SEQ ID NO: 6) |
| | FPPKPKDTLYITREPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APIEKTISKAKGQPREPQVYTLP | |
| | PSREEMTKNQVSLTCLVKGFYP | |
| | SDIAVEWESNGQPENNYKTTPP | |
| | VLDSDGSFFLYSKLTVDKSRWQ | |
| | QGNVFSCSVMHEALHNHYTQK | |
| | SLSLSPGK (SEQ ID NO: 5) | |

TABLE 4
VH and VL AA Sequences of
of the Lead anti-IL-1β mAbs

| Name | VH AA Seq | VL AA Seq |
|---|---|---|
| 05H21A | QVTLRESGPALVKPTQTL | QSVLTQPPSVSEAPRQRV |
| | TLTCTFSGFSLSTSGMWV | TISCSGSSSNIGDNAVNW |
| | SWIRQPPGKALEWLALID | YQQLPGKAPKLLIYNDDL |
| | WGDDKYYTTSLKTRLTIS | LSSGVSDRFSGSKSGTSA |
| | KDTSKNQVVLTMTNMDPV | SLAISGLQSEDEADYYCA |
| | DTATYYCARMREGSRAFD | AWDDSLNGPVFGGGTKLT |
| | IWGQGTVVTVSS | VL |
| | (SEQ ID NO: 7) | (SEQ ID NO: 8) |
| 15N14A | QVQLQESGPGLVKPSETL | QAVLTQPSSLSASPGASA |
| | SLTCTVSGGSISSYYWTW | SLTCTLRSGINVGTYRIY |
| | IRQPAGKGLEWIGRIDSS | WYQQKPGSPPQYLLSYKS |
| | GSSKYNPTLKSRVTMSVD | DSDKQQGSGVPSRFSGSK |
| | TSKNQFSLKLSSVTAADT | DASANVGILLISGLQSED |
| | AVYYCARGDSGYDWAFDY | EADYYCMIWHSSAWVFGG |
| | WGQGTLVTVSS | GTKLTVL |
| | (SEQ ID NO: 9) | (SEQ ID NO: 10) |
| 08F17A | EVQLVESGGGLVTPGGSL | DIQMTQSPSSLSASVGDR |
| | RLSCAASGFTFSGYSMNW | VTITCRASQGISYYLAWY |
| | VRQAPGKGLEWVSSISSS | QQKPGKVPKLLISAEFTL |
| | SGYIYYADSVKGRFTISR | QSGVPSRFSGSGSGTDFT |
| | DNAKNSLYLQMNSLRAED | LTISSLQPEDVATYYCQK |
| | TAVYYCAREYWGSGFDYW | YNTAPRTFGQGTKVEIK |
| | GQGTLVTVSS | (SEQ ID NO: 12) |
| | (SEQ ID NO: 11) | |

TABLE 5
Kabat CDR AA Sequences of the Lead anti-IL-1β mAbs

| Name | HCDR1 | HCDR2 | HCDR3 | LCDR1 | LCDR2 | LCDR3 |
|---|---|---|---|---|---|---|
| 05H21A | TSGMWV | LIDWGD | MREGSR | SGSSSNI | NDDLLSS | AAWDDS |
| | S (SEQ ID | DKYYTTS | AFDI | GDNAVN | (SEQ ID | LNGPV |
| | NO: 13) | LKT (SEQ | (SEQ ID | (SEQ ID | NO: 17) | (SEQ ID |
| | | ID NO: 14) | NO: 15) | NO: 16) | | NO: 18) |
| 15N14A | SYYWT | RIDSSGS | GDSGYD | TLRSGIN | YKSDSD | MIWHSS |
| | (SEQ ID | SKYNPTL | WAFDY | VGTYRIY | KQQGS | AWV |
| | NO: 19) | KS (SEQ | (SEQ ID | (SEQ ID | (SEQ ID | (SEQ ID |
| | | ID NO: 20) | NO: 21) | NO: 22) | NO: 23) | NO: 24) |
| 08F17A | GYSMN | SISSSSGY | EYWGSG | RASQGIS | AEFTLQS | QKYNTA |
| | (SEQ ID | IYYADSV | FDY (SEQ | YYLA | (SEQ ID | PRT (SEQ |
| | NO: 25) | KG (SEQ | ID NO: 27) | (SEQ ID | NO: 29) | ID NO: 30) |
| | | ID NO: 26) | | NO: 28) | | |

TABLE 6
Chothia CDR AA Sequences of the Lead anti-IL-1β mAbs

| Name | HCDR1 | HCDR2 | HCDR3 | LCDR1 | LCDR2 | LCDR3 |
|---|---|---|---|---|---|---|
| 05H21A | GFSLSTS | DWGDD | MREGSR | SGSSSNI | NDDLLSS | AAWDDS |
| | GM (SEQ | (SEQ ID | AFDI | GDNAVN | (SEQ ID | LNGPV |
| | ID NO: 31) | NO: 32) | (SEQ ID | (SEQ ID | NO: 35) | (SEQ ID |
| | | | NO: 33) | NO: 34) | | NO: 36) |
| 15N14A | GGSISSY | DSSGS | GDSGYD | TLRSGIN | YKSDSD | MIWHSS |
| | (SEQ ID | (SEQ ID | WAFDY | VGTYRIY | KQQGS | AWV |
| | NO: 37) | NO: 38) | (SEQ ID | (SEQ ID | (SEQ ID | (SEQ ID |
| | | | NO: 39) | NO: 40) | NO: 41) | NO: 42) |
| 08F17A | GFTFSGY | SSSSGY | EYWGSG | RASQGIS | AEFTLQS | QKYNTA |
| | (SEQ ID | (SEQ ID | FDY (SEQ | YYLA | (SEQ ID | PRT (SEQ |
| | NO: 43) | NO: 44) | ID NO: 45) | (SEQ ID | NO: 47) | ID NO: 48) |
| | | | | NO: 46) | | |

TABLE 7
AbM CDR AA Sequences of the Lead anti-IL-1β mAbs

| Name | HCDR1 | HCDR2 | HCDR3 | LCDR1 | LCDR2 | LCDR3 |
|---|---|---|---|---|---|---|
| 05H21A | GFSLSTS | LIDWGD | MREGSR | SGSSSNI | NDDLLSS | AAWDDS |
| | GMWVS | DKY (SEQ | AFDI | GDNAVN | (SEQ ID | LNGPV |
| | (SEQ ID | ID NO: 50) | (SEQ ID | (SEQ ID | NO: 53) | (SEQ ID |
| | NO: 49) | | NO: 51) | NO: 52) | | NO: 54) |
| 15N14A | GGSISSY | RIDSSGS | GDSGYD | TLRSGIN | YKSDSD | MIWHSS |
| | YWT | SK (SEQ | WAFDY | VGTYRIY | KQQGS | AWV |
| | (SEQ ID | ID NO: 56) | (SEQ ID | (SEQ ID | (SEQ ID | (SEQ ID |
| | NO: 55) | | NO: 57) | NO: 58) | NO: 59) | NO: 60) |
| 08F17A | GFTFSGY | SISSSSGY | EYWGSG | RASQGIS | AEFTLQS | QKYNTA |
| | SMN (SEQ | IY (SEQ | FDY (SEQ | YYLA | (SEQ ID | PRT (SEQ |
| | ID NO: 61) | ID NO: 62) | ID NO: 63) | (SEQ ID | NO: 65) | ID NO: 66) |
| | | | | NO: 64) | | |

TABLE 8
Contact CDR AA Sequences of the Lead anti-IL-1β mAbs

| Name | HCDR1 | HCDR2 | HCDR3 | LCDR1 | LCDR2 | LCDR3 |
|---|---|---|---|---|---|---|
| 05H21A | STSGMW | WLALID | ARMREG | IGDNAVN | LLIYNDD | AAWDDS |
| | VS (SEQ | WGDDKY | SRAFD | WY (SEQ | LLS (SEQ | LNGP |
| | ID NO: | (SEQ ID | (SEQ ID | ID NO: 70) | ID NO: 71) | (SEQ ID |
| | 67) | NO: 68) | NO: 69) | | | NO: 72) |
| 15N14A | SSYYWT | WIGRIDS | ARGDSG | NVGTYRI | YLLSYKS | MIWHSS |
| | (SEQ ID | SGSSK | YDWAFD | YWY | DSDKQQ | AW (SEQ |
| | NO: 73) | (SEQ ID | (SEQ ID | (SEQ ID | G (SEQ ID | ID NO: 78) |
| | | NO: 74) | NO: 75) | NO: 76) | NO: 77) | |
| 08F17A | SGYSMN | WVSSISS | AREYWG | SYYLAW | LLISAEFT | QKYNTA |
| | (SEQ ID | SSGYIY | SGFD | Y (SEQ ID | LQ (SEQ | PR (SEQ |
| | NO: 79) | (SEQ ID | (SEQ ID | NO: 82) | ID NO: 83) | ID NO: 84) |
| | | NO: 80) | NO: 81) | | | |

TABLE 9
IMGT CDR AA Sequences of the Lead anti-IL-1β mAbs

| Name | HCDR1 | HCDR2 | HCDR3 | LCDR1 | LCDR2 | LCDR3 |
|---|---|---|---|---|---|---|
| 05H21A | GFSLSTS | IDWGDD | ARMREG | SSNIGDN | NDD (SEQ | AAWDDS |
| | GMW | K (SEQ ID | SRAFDI | A (SEQ ID | ID NO: 89) | LNGPV |
| | (SEQ ID | NO: 86) | (SEQ ID | NO: 88) | | (SEQ ID |
| | NO: 85) | | NO: 87) | | | NO: 90) |
| 15N14A | GGSISSY | IDSSGSS | ARGDSG | SGINVGT | YKSDSD | MIWHSS |
| | Y (SEQ ID | (SEQ ID | YDWAFD | YR (SEQ | K (SEQ ID | AWV |
| | NO: 91) | NO: 92) | Y (SEQ ID | ID NO: 94) | NO: 95) | (SEQ ID |
| | | | NO: 93) | | | NO: 96) |
| 08F17A | GFTFSGY | ISSSSGYI | AREYWG | QGISYY | AEF (SEQ | QKYNTA |
| | S (SEQ ID | (SEQ ID | SGFDY | (SEQ ID | ID NO: | PRT (SEQ |
| | NO: 97) | NO: 98) | (SEQ ID | NO: 100) | 101) | ID NO: |
| | | | NO: 99) | | | 102) |

7.1.1 Molecule Design of 05H21A, 15N14A and 08F17A

05H21A, 15N14A and 08F17A are immunoglobulin G1 (IgG1) monoclonal antibodies (mAb) that bind to human IL-1β. The antibody features mutations of M252Y, S254T, and T256E (YTE, EU numbering) in the constant region to enhance interaction with the neonatal Fc receptors (FcRn) at pH˜6, thereby enhancing FcRn-mediated endosomal recycling, leading to longer serum exposure (Dall'Acqua et al., J Biol Chem, 2006. 281(33): 23514-241; Dall'Acqua et al., J Immunol, 2002. 169(9): 5171-80). These mAbs were developed for targeting human IL-1β, and blocking its binding to IL-1R1, and inhibiting IL-1β signaling, which is a inflammatory pathway that has been linked to cancer (Ridker, P. M. et al., Lancet, 2017. 390(10105): 1833-1842).

7.1.2 Source of Coding Sequence

05H21A, 15N14A and 08F17A were obtained by immunizing transgenic mice possessing a human antibody repertoire (Ablexis®) with recombinant DNA encoding for hIL-1β D145K. These three lead antibodies were sub-cloned into the human IgG1-YTE constant region and recombinantly expressed.

7.1.3 Sequence and Structure

7.1.3.1 Amino Acid Sequence of the 05H21A, 15N14A and 08F17A

The amino acid sequence for the 05H21A, 15N14A and 08F17A HC and LC, as deduced from their cDNA sequence, and confirmed by peptide mapping and mass spectrometry, is shown in Table 3. The 3 complementarity-determining regions (CDRs) in each chain are shown in Table 5 to Table 9. The VL and VH amino acid sequences of 05H21A, 15N14A and 08F17A are shown in Table 4. The Glutamine (Q) residue at position 1 of the HC and Glutamine (Q) at position 1 of the LC constitute the N-termini of the mature chains of 05H21A and 15N14A. The Glutamic Acid (E) residue at position 1 of the HC and Aspartic Acid (D) at position 1 of the LC constitute the N-termini of the mature chains of 08F17A. The YTE mutations in the heavy chain constant domains of 05H21A, 15N14A, and 08F17A are bolded and underlined in Table 1.

7.1.3.2 In Silico Immunogenicity Risk Assessment by EpiVax EpiMatrix for 05H21A, 15N14A and 08F17A Variable Domains

The mAb variable region sequences were analyzed for potential immunogenicity using the T-regulatory (T_{reg}) adjusted scores from the EpiVax EpiMatrix in silico immunogenicity prediction program (De Groot et al., Clin Immunol, 2009. 131(2): 189-201). The EpiVax Epimatrix program calculates the binding potential to the most common HLA molecules within each of the “supertypes” or groupings. The report provides results that are representative of >90% of the worldwide human population without the necessity of testing each haplotype individually. The EpiVax score is calculated by aggregating the EpiMatrix scores of all predicted T-cell epitopes contained within a given protein sequence and adjusting for expected T-cell epitope content and protein length. The EpiVax score and EpiVax interpretation are shown as below in Table 10.

TABLE 10
Immunogenicity assessment for 05H21A (anti-hu IL-1β)

| Ab Name | Chain Information | EpiVax score |
|---|---|---|
| 05H21A | Light chain VL | −61.54 |
| | Heavy Chain VH | −35.07 |
| 15N14A | Light chain VL | 3.31 |
| | Heavy Chain VH | 14.84 |
| 08F17A | Light chain VL | −42.83 |
| | Heavy Chain VH | −37.91 |

### 7.2 Example 2: Generation of the Expression Construct of 05H21A, 15N14A and 08F17A

The cDNA encoding the 05H21A, 15N14A and 08F17A antibodies were synthesized and subcloned into a Leap-In Transposase® glutamine synthetase expression vector backbone at ATUM (CA, USA). The dual-gene expression plasmids were then transferred to the Manufacturing Plasmid Generation Group of JRD (PA, USA) and their sequence was confirmed at NeoGenomics Laboratories (CA, USA).

The primary transcript nucleotide sequence for the Heavy Chain, and Light Chain genes are listed in Table 11.

TABLE 11
HC and LC Nucleotide Sequences
of 05H21A, 15N14A and 08F17A

| Name | LC Nucleotide Seq | HC Nucleotide Seq |
|---|---|---|
| 05H21A | CAGTCTGTGCTGACCCAGCCTCC | CAAGTGACCCTGAGAGAGTCT |
| | ATCTGTGTCTGAGGCCCCTAGAC | GGACCCGCTCTGGTCAAGCCC |
| | AGAGAGTGACCATCTCCTGCTC | ACACAGACCCTGACACTGACC |
| | CGGCTCCTCCTCTAACATCGGCG | TGCACCTTCTCCGGCTTCTCCC |
| | ATAACGCCGTGAACTGGTATCA | TGTCCACCTCTGGAATGTGGG |
| | GCAGCTGCCTGGCAAGGCCCCT | TGTCCTGGATCAGACAGCCTC |
| | AAACTGCTGATCTACAACGACG | CTGGCAAGGCACTGGAATGGC |
| | ACCTGCTGTCCTCTGGCGTGTCC | TGGCTCTGATCGATTGGGGCG |
| | GACAGATTCTCCGGCTCTAAGTC | ACGACAAGTACTACACCACCA |
| | TGGCACCTCCGCCAGCCTGGCT | GCCTGAAAACCCGGCTGACCA |
| | ATCTCTGGATTGCAGTCTGAGG | TCTCCAAGGACACCTCCAAGA |
| | ACGAGGCCGACTACTACTGTGC | ACCAGGTGGTGCTGACCATGA |
| | CGCCTGGGACGATTCTCTGAAC | CCAACATGGACCCTGTGGACA |
| | GGCCCTGTTTTTGGCGGAGGCA | CCGCCACCTACTACTGCGCCA |
| | CCAAGCTGACAGTCCTGGGTCA | GAATGAGAGAGGGATCCAGAG |
| | GCCCAAGGCTGCACCCAGTGTC | CCTTCGATATCTGGGGCCAGG |
| | ACTCTGTTCCCGCCCTCCTCTGA | GAACCGTGGTCACCGTTTCTT |
| | GGAGCTTCAAGCCAACAAGGCC | CTGCCTCCACCAAGGGCCCAT |
| | ACACTGGTGTGTCTCATAAGTG | CGGTCTTCCCCCTGGCACCCTC |
| | ACTTCTACCCGGGAGCCGTGAC | CTCCAAGAGCACCTCTGGGGG |
| | AGTGGCCTGGAAGGCCGATAGC | CACAGCGGCCCTGGGCTGCCT |
| | AGCCCCGTCAAGGCGGGAGTCG | GGTCAAGGACTACTTCCCCGA |
| | AAACCACCACACCCTCCAAACA | ACCGGTGACGGTGTCGTGGAA |
| | AAGCAACAACAAGTACGCGGCC | CTCAGGCGCCCTGACCAGCGG |
| | AGCAGCTATCTGAGCCTGACGC | CGTGCACACCTTCCCGGCTGTC |
| | CTGAGCAGTGGAAGTCCCACAG | CTACAGTCCTCAGGACTCTACT |
| | AAGCTACAGCTGCCAGGTCACG | CCCTCAGCAGCGTGGTGACCG |
| | CATGAAGGGAGCACCGTGGAGA | TGCCCTCCAGCAGCTTGGGCA |
| | AGACAGTGGCCCCTACAGAATG | CCCAGACCTACATCTGCAACG |
| | TTCA (SEQ ID NO: 103) | TGAATCACAAGCCCAGCAACA |
| | | CCAAGGTGGACAAGAAAGTTG |
| | | AGCCCAAATCTTGTGACAAAA |
| | | CTCACACATGCCCACCGTGCC |
| | | CAGCACCTGAACTCCTGGGGG |
| | | GACCGTCAGTCTTCCTCTTCCC |
| | | CCCAAAACCCAAGGACACCCT |
| | | CTACATCACCCGGGAGCCTGA |
| | | GGTCACATGCGTGGTGGTGGA |
| | | CGTGAGCCACGAAGACCCTGA |
| | | GGTCAAGTTCAACTGGTACGT |
| | | GGACGGCGTGGAGGTGCATAA |
| | | TGCCAAGACAAAGCCGCGGGA |
| | | GGAGCAGTACAACAGCACGTA |
| | | CCGTGTGGTCAGCGTCCTCAC |
| | | CGTCCTGCACCAGGACTGGCT |
| | | GAATGGCAAGGAGTACAAGTG |
| | | CAAGGTGTCCAACAAAGCCCT |
| | | CCCAGCCCCCATCGAGAAAAC |
| | | CATCTCCAAAGCCAAAGGGCA |
| | | GCCCCGAGAACCACAGGTGTA |
| | | CACCCTGCCCCCATCCCGGGA |
| | | GGAGATGACCAAGAACCAGGT |
| | | CAGCCTGACCTGCCTGGTCAA |
| | | AGGCTTCTATCCCAGCGACAT |
| | | CGCCGTGGAGTGGGAGAGCAA |
| | | TGGGCAGCCGGAGAACAACTA |
| | | CAAGACCACGCCTCCCGTGCT |
| | | GGACTCCGACGGCTCCTTCTTC |
| | | CTCTACAGCAAGCTCACCGTG |
| | | GACAAGAGCAGATGGCAGCA |
| | | GGGGAACGTCTTCTCATGCTC |
| | | CGTGATGCATGAGGCTCTGCA |
| | | CAACCACTACACGCAGAAGTC |
| | | TCTCTCCCTGTCTCCGGGAAAA |
| | | (SEQ ID NO: 104) |
| 15N14A | CAGGCTGTTCTGACCCAGCCTA | CAGGTTCAGCTGCAAGAGTCT |
| | GCTCTCTGTCTGCTTCTCCTGGC | GGACCCGGCCTGGTCAAGCCT |
| | GCTTCCGCCTCTCTGACCTGCAC | TCCGAGACACTGTCTCTGACCT |
| | ACTGAGATCCGGCATCAACGTG | GCACCGTGTCTGGCGGCTCCA |
| | GGCACCTACCGGATCTACTGGT | TCTCCTCTTACTATTGGACCTG |
| | ATCAGCAGAAGCCTGGCAGCCC | GATCAGACAGCCTGCCGGCAA |
| | TCCTCAGTACCTGCTGTCCTACA | AGGCCTGGAATGGATCGGCAG |
| | AGTCCGACTCCGACAAGCAGCA | AATCGACTCCTCCGGCTCCTCC |
| | AGGCTCTGGCGTGCCCTCTAGAT | AAGTACAACCCCACACTGAAG |
| | TCTCCGGCTCTAAGGACGCCTCC | TCCAGAGTGACCATGTCCGTG |
| | GCCAATGTGGGCATCCTGCTGA | GACACCTCCAAGAACCAGTTC |
| | TCTCTGGCCTGCAGTCTGAGGAC | TCCCTGAAGCTGTCCTCCGTGA |
| | GAGGCCGACTACTACTGCATGA | CCGCTGCTGATACCGCCGTGT |
| | TCTGGCACTCCTCCGCCTGGGTT | ACTACTGTGCCAGAGGCGACT |
| | TTCGGAGGCGGAACAAAGCTGA | CTGGATACGACTGGGCCTTTG |
| | CAGTCCTGGGTCAGCCCAAGGC | ACTATTGGGGCCAGGGCACAC |
| | TGCACCCAGTGTCACTCTGTTCC | TGGTCACCGTTTCTTCTGCCTC |
| | CGCCCTCCTCTGAGGAGCTTCAA | CACCAAGGGCCCATCGGTCTT |
| | GCCAACAAGGCCACACTGGTGT | CCCCCTGGCACCCTCCTCCAA |
| | GTCTCATAAGTGACTTCTACCCG | GAGCACCTCTGGGGGCACAGC |
| | GGAGCCGTGACAGTGGCCTGGA | GGCCCTGGGCTGCCTGGTCAA |
| | AGGCCGATAGCAGCCCCGTCAA | GGACTACTTCCCCGAACCGGT |
| | GGCGGGAGTCGAAACCACCACA | GACGGTGTCGTGGAACTCAGG |
| | CCCTCCAAACAAAGCAACAACA | CGCCCTGACCAGCGGCGTGCA |
| | AGTACGCGGCCAGCAGCTATCT | CACCTTCCCGGCTGTCCTACAG |
| | GAGCCTGACGCCTGAGCAGTGG | TCCTCAGGACTCTACTCCCTCA |
| | AAGTCCCACAGAAGCTACAGCT | GCAGCGTGGTGACCGTGCCCT |
| | GCCAGGTCACGCATGAAGGGAG | CCAGCAGCTTGGGCACCCAGA |
| | CACCGTGGAGAAGACAGTGGCC | CCTACATCTGCAACGTGAATC |
| | CCTACAGAATGTTCA (SEQ ID | ACAAGCCCAGCAACACCAAGG |
| | NO: 105) | TGGACAAGAAAGTTGAGCCCA |
| | | AATCTTGTGACAAAACTCACA |
| | | CATGCCCACCGTGCCCAGCAC |
| | | CTGAACTCCTGGGGGGACCGT |
| | | CAGTCTTCCTCTTCCCCCCAAA |
| | | ACCCAAGGACACCCTCTACAT |
| | | CACCCGGGAGCCTGAGGTCAC |
| | | ATGCGTGGTGGTGGACGTGAG |
| | | CCACGAAGACCCTGAGGTCAA |
| | | GTTCAACTGGTACGTGGACGG |
| | | CGTGGAGGTGCATAATGCCAA |
| | | GACAAAGCCGCGGGAGGAGC |
| | | AGTACAACAGCACGTACCGTG |
| | | TGGTCAGCGTCCTCACCGTCCT |
| | | GCACCAGGACTGGCTGAATGG |
| | | CAAGGAGTACAAGTGCAAGGT |
| | | GTCCAACAAAGCCCTCCCAGC |
| | | CCCCATCGAGAAAACCATCTC |
| | | CAAAGCCAAAGGGCAGCCCCG |
| | | AGAACCACAGGTGTACACCCT |
| | | GCCCCCATCCCGGGAGGAGAT |
| | | GACCAAGAACCAGGTCAGCCT |
| | | GACCTGCCTGGTCAAAGGCTT |
| | | CTATCCCAGCGACATCGCCGT |
| | | GGAGTGGGAGAGCAATGGGC |
| | | AGCCGGAGAACAACTACAAGA |
| | | CCACGCCTCCCGTGCTGGACT |
| | | CCGACGGCTCCTTCTTCCTCTA |
| | | CAGCAAGCTCACCGTGGACAA |
| | | GAGCAGATGGCAGCAGGGGA |
| | | ACGTCTTCTCATGCTCCGTGAT |
| | | GCATGAGGCTCTGCACAACCA |
| | | CTACACGCAGAAGTCTCTCTC |
| | | CCTGTCTCCGGGAAAA |
| | | (SEQ ID NO: 106) |
| 08F17A | GACATCCAGATGACCCAGTCTC | GAGGTGCAGCTGGTTGAATCT |
| | CATCCTCTCTGTCCGCTTCTGTG | GGCGGAGGACTGGTTACCCCT |
| | GGCGACAGAGTGACCATCACCT | GGCGGATCTCTGAGACTGTCT |
| | GTAGAGCCTCTCAGGGCATCTC | TGTGCCGCCTCTGGCTTCACCT |
| | CTACTACCTGGCCTGGTATCAGC | TCTCCGGCTACTCTATGAACTG |
| | AGAAACCCGGCAAGGTGCCCAA | GGTCCGACAGGCTCCTGGCAA |
| | GCTGCTGATCTCTGCTGAGTTCA | AGGCCTGGAATGGGTGTCCTC |
| | CCCTGCAGTCTGGCGTGCCCTCT | CATCTCCTCCAGCAGCGGCTA |
| | AGATTCTCCGGCTCTGGCTCTGG | CATCTACTACGCCGACTCCGT |
| | CACCGACTTTACCCTGACAATCT | GAAGGGCAGATTCACCATCTC |
| | CCAGCCTGCAGCCTGAGGATGT | CAGAGACAACGCCAAGAACTC |
| | GGCCACCTACTACTGCCAGAAG | CCTGTACCTGCAGATGAACAG |
| | TACAACACCGCTCCTCGGACCTT | CCTGAGAGCCGAGGACACCGC |
| | TGGCCAGGGCACCAAGGTGGAA | CGTGTACTACTGTGCCAGAGA |
| | ATCAAGCGTACTGTGGCTGCAC | GTATTGGGGCTCCGGCTTCGA |
| | CATCTGTCTTCATCTTCCCGCCA | TTATTGGGGCCAAGGAACACT |
| | TCTGATGAGCAGTTGAAATCTG | GGTCACCGTGTCCTCTGCCTCC |
| | GAACTGCCTCTGTTGTGTGCCTG | ACCAAGGGCCCATCGGTCTTC |
| | CTGAATAACTTCTATCCCAGAG | CCCCTGGCACCCTCCTCCAAG |
| | AGGCCAAAGTACAGTGGAAGGT | AGCACCTCTGGGGGCACAGCG |
| | GGATAACGCCCTCCAATCGGGT | GCCCTGGGCTGCCTGGTCAAG |
| | AACTCCCAGGAGAGTGTCACAG | GACTACTTCCCCGAACCGGTG |
| | AGCAGGACAGCAAGGACAGCAC | ACGGTGTCGTGGAACTCAGGC |
| | CTACAGCCTCAGCAGCACCCTG | GCCCTGACCAGCGGCGTGCAC |
| | ACGCTGAGCAAAGCAGACTACG | ACCTTCCCGGCTGTCCTACAGT |
| | AGAAACACAAAGTCTACGCCTG | CCTCAGGACTCTACTCCCTCAG |
| | CGAAGTCACCCATCAGGGCCTG | CAGCGTGGTGACCGTGCCCTC |
| | AGCTCGCCCGTCACAAAGAGCT | CAGCAGCTTGGGCACCCAGAC |
| | TCAACAGGGGAGAGTGT (SEQ | CTACATCTGCAACGTGAATCA |
| | ID NO: 107) | CAAGCCCAGCAACACCAAGGT |
| | | GGACAAGAAAGTTGAGCCCAA |
| | | ATCTTGTGACAAAACTCACAC |
| | | ATGCCCACCGTGCCCAGCACC |
| | | TGAACTCCTGGGGGGACCGTC |
| | | AGTCTTCCTCTTCCCCCCAAAA |
| | | CCCAAGGACACCCTCTACATC |
| | | ACCCGGGAGCCTGAGGTCACA |
| | | TGCGTGGTGGTGGACGTGAGC |
| | | CACGAAGACCCTGAGGTCAAG |
| | | TTCAACTGGTACGTGGACGGC |
| | | GTGGAGGTGCATAATGCCAAG |
| | | ACAAAGCCGCGGGAGGAGCA |
| | | GTACAACAGCACGTACCGTGT |
| | | GGTCAGCGTCCTCACCGTCCT |
| | | GCACCAGGACTGGCTGAATGG |
| | | CAAGGAGTACAAGTGCAAGGT |
| | | GTCCAACAAAGCCCTCCCAGC |
| | | CCCCATCGAGAAAACCATCTC |
| | | CAAAGCCAAAGGGCAGCCCCG |
| | | AGAACCACAGGTGTACACCCT |
| | | GCCCCCATCCCGGGAGGAGAT |
| | | GACCAAGAACCAGGTCAGCCT |
| | | GACCTGCCTGGTCAAAGGCTT |
| | | CTATCCCAGCGACATCGCCGT |
| | | GGAGTGGGAGAGCAATGGGC |
| | | AGCCGGAGAACAACTACAAGA |
| | | CCACGCCTCCCGTGCTGGACT |
| | | CCGACGGCTCCTTCTTCCTCTA |
| | | CAGCAAGCTCACCGTGGACAA |
| | | GAGCAGATGGCAGCAGGGGA |
| | | ACGTCTTCTCATGCTCCGTGAT |
| | | GCATGAGGCTCTGCACAACCA |
| | | CTACACGCAGAAGTCTCTCTC |
| | | CCTGTCTCCGGGAAAA |
| | | (SEQ ID NO: 108) |

The mature amino acid sequences for 05H21A, 15N14A and 08F17A, as deduced from the cDNA sequence and confirmed by peptide mapping and mass spectrometry, is shown above in Table 3.

### 7.3 Example 3: Biophysical Assessment of 05H21A, 15N14A, and 08F17A

7.3.1 Binding Affinity of 05H21A, 15N14A, and 08F17A to Human IL-β by SPR

Affinity assessment of 05H21A, 15N14A, and 08F17A against human IL-1β was performed by Surface Plasmon Resonance (SPR) using a Biacore 8k. Cross-reactivity of the same antibody panel was also assessed against cyno, mouse, rat and pig IL-1β.

SPR is a label-free technique to study the strength of an interaction between two binding partners by measuring the change in mass upon complex formation and dissociation. Briefly, antibodies were immobilized on a sensor chip which was coupled with goat anti-human Fc (GAH-Fc C1 sensor chip). Soluble IL-1β was flowed over the immobilized antibody and association/dissociation responses were monitored. Kinetic information (on-rate and off-rate constants) were extracted by fitting sensorgrams to the 1:1 Langmuir model. Binding affinity (K_{D}) were reported as the ratio of rate constants (k_{off}/kₒₙ). Binding assessments results are shown in Table 12.

05H21A, 15N14A, and 08F17A bind human IL-1β with high affinities of ˜20 pM, ˜64 pM, and ˜180 pM, respectively. 05H21A, 15N14A, and 08F17A also have cross reactivity to cyno IL-1β with affinities of ˜22 pM, ˜70 pM, and ˜1.7 nM. All three Abs showed no or weak cross reactivity to mouse, rat, and pig IL-1β.

TABLE 12
Binding Assessments of 05H21A, 15N14A and 08F17A

| mAb Characteristic | 05H21A | 15N14A | 08F17A |
|---|---|---|---|
| Binding Affinity | KD (M) = 1.97E−11 | KD (M) = 6.4E−11 | KD (M) = 1.8E−10 |
| to human IL-β | ka (1/Ms) = 2.14E+07 | ka (1/Ms) = 2.20E+06 | ka (1/Ms) = 8.55E−05 |
| | kd (1/s) = 4.22E−04 | kd (1/s) = 1.41E−04 | kd (1/s) = 1.82E−10 |
| Binding Affinity | KD (M) = 2.20E−11M | KD = 7.0E−11 | KD (M) = 1.71E−09 |
| to Cyno IL-β | ka (1/Ms) = 1.58E+07 | ka (1/Ms) = 3.01E+06 | ka (1/Ms) = 1.72E+05 |
| | kd (1/s) = 3.48E−04 | kd (1/s) = 2.09E−04 | kd (1/s) = 2.95E−04 |
| Binding Affinity | weak binding | No binding | No binding |
| to mouse IL-β | KD > 100 nM | | |
| Binding Affinity | weak binding, | KD = 7.45E−08 | No binding |
| to rat IL-β | KD > 100 nM | ka (1/Ms) = 7.71E+05 | |
| | | kd (1/s) = 5.74E−02 | |
| Binding Affinity | No binding | No binding | No binding |
| to pig IL-β | | | |

7.3.2 Epitope Identification for 05H21A, 15N14A, and 08F17A by HDX

The epitope on IL-1β was determined by hydrogen-deuterium exchange mass spectrometry (HDX-MS). The epitope mapping of select antibodies on IL-1β using the hydrogen-deuterium exchange-based LC-MS is shown in FIG. 1.

On-Exchange Experiment for HDX-MS. On-exchange reaction was initiated by mixing 10 μL of 10 μM IL-1β (R&D Systems), which was residues 117-269 of hIL-1β (Uniprot ID P01584; SEQ ID NO: 109), with or without 1.2 molar-excess of ligand and 30 μL of H₂O or a deuterated buffer (20 mM MES, pH 6.4, 150 mM NaCl in 95% D20 or 20 mM Tris, pH 8.4, 150 mM NaCl in 95% D20). The reaction mixture was incubated for 15, 50, 150, 500, or 1,500 s at 1.2° C. The on-exchanged solution was quenched by the addition of chilled 40 μL of 1.6 M guanidine hydrochloride, 0.8% formic acid and immediately analyzed.

IL-1β (Uniprot ID P01584) (SEQ ID NO: 109):
MAEVPELASEMMAYYSGNEDDLFFEADGPKQMKCSFQDLDLCPLDGGI
QLRISDHHYSKGFRQAASVVVAMDKLRKMLVPCPQTFQENDLSTFFPF
IFEEEPIFFDTWDNEAYVHDAPVRSLNCTLRDSQQKSLVMSGPYELKA
LHLQGQDMEQQVVFSMSFVQGEESNDKIPVALGLKEKNLYLSCVLKDD
KPTLQLESVDPKNYPKKKMEKRFVFNKIEINNKLEFESAQFPNWYIST
SQAENMPVFLGGTKGGQDITDFTMQFVSS

General Procedure for HDX-MS Data Acquisition. HDX-MS sample preparation was performed with automated HDx system (LEAP Technologies, Morrisville, N.C.). The columns and pump were protease, protease type XIII (protease from Aspergillus saitoi, type XIII)/pepsin column (w/w, 1:1; 2.1×30 mm) (NovaBioAssays Inc., Woburn, Mass.); trap, ACQUITY UPLC BEH C18 VanGuard Pre-column (2.1×5 mm) (Waters, Milford, Mass.), analytical, Accucore C18 (2.1×100 mm) (Thermo Fisher Scientific, Waltham, Mass.); and LC pump, VH-P10-A (Thermo Fisher Scientific). The loading pump (from the protease column to the trap column) was set at 600 μL/min with 0.1% aqueous formic acid. The gradient pump (from the trap column to the analytical column) was set from 9% to 33% acetonitrile in 0.1% aqueous formic acid in 20 min at 100 μL/min.

MS Data Acquisition. Mass spectrometric analyses were carried out using an LTQ™ Orbitrap Fusion Lumos mass spectrometer (Thermo Fisher Scientific) with the capillary temperature at 275° C., resolution 120,000, and mass range (m/z) 300-1,500.

HDX-MS Data Extraction. BioPharma Finder 2.0 (Thermo Fisher Scientific) was used for the peptide identification of non-deuterated samples prior to the HDX experiments. HDExaminer version 2.5 (Sierra Analytics, Modesto, Calif.) was used to extract centroid values from the MS raw data files for the HDX experiments.

HDX-MS Data Analysis. The extracted HDX-MS data were further analyzed in Excel. All exchange time points (at pH 6.4 or pH 8.4 at 23° C.) were converted to the equivalent time points at pH 7.4 and 23° C. (e.g., 15 s at pH 6.4 at 23° C. is equivalent of 1.5 s at pH 7.4 at 23° C. (Table 13).

TABLE 13
HDX reaction conditions and exchange times versus
exchange times corrected to pH 7.4 and 23° C.

| Time adjusted to | pH 6.4 | pH 8.4 |
|---|---|---|
| pH 7.4, 23° C. (s) | 23° C. (s) | 23° C. (s) |
| 0.15 | — | — |
| 0.5 | — | — |
| 1.5 | 15 | — |
| 5 | 50 | — |
| 15 | 150 | — |
| 50 | 500 | — |
| 150 | 1,500 | 15 |
| 500 | — | 50 |
| 1,500 | — | 150 |
| 5,000 | — | 500 |
| 15,000 | — | 1,500 |

Results. The strong epitopes of IL-1β (ΔΔG upon binding ≤−2 kcal/mol) against 05H21A were residues 119 (V), 161-162 (SF) and 164-169 (QGEESN). The weak epitopes (−2<ΔΔG upon binding ≤−1 kcal/mol) were residues 120-123 (RSLN) and 157-160 (VFSM).

The strong epitopes of IL-1β against 08F17A were residues 147-156 (LQGQDMEQQV) and 264-267 (MQFV).

The strong epitopes of IL-1β against 15N14A were residues 164-169 (QGEESN). The weak epitopes were residues 158-162 (FSMSF).

7.3.3 Conformational Stability of 05H21A, 15N14A, and 08F17A by DSC

Conformational stability of 05H21A, 15N14A, and 08F17A was determined by differential scanning calorimetry (DSC). The Differential scanning calorimetry experiment was carried out at 1 mg/mL in 10 mM sodium acetate pH 5.5 using Microcal VP-DSC Malvern instrument (Northampton, Mass., USA). Thermal scans were performed from 25° C. to 95° C. using 60° C./h scan rate with 15 min prescan thermostat. Each antibody was measure in duplicate with buffer/buffer blank run between each sample run. Data analysis was performed using MicroCal Origin Version 7 software and transition temperatures (Tms) were reported in Table 14. 05H21A thermogram revealed 3 transition temperatures at 62.2° C. (Tm1), 77.9° C. (Tm2) and 84.0° C. (Tm3). 15N14A thermogram revealed 4 transition temperatures at 64.7° C. (Tm1), 68.0° C. (Tm2), 76.9° C. (Tm3), and 83.3° C. (Tm4). 08F17A thermogram revealed 3 transition temperatures at 62.8° C. (Tm1), 75.5° C. (Tm2) and 83.3° C. (Tm3)

TABLE 14
Transition temperatures for 05H21A, 15N14A, and 08F17A

| mAb Characteristic | 05H21A | 15N14A | 08F17A | Notes |
|---|---|---|---|---|
| Differential Scanning | Tm₁ = 62.2° C. | Tm₁ = 63.2° C. | Tm₁ = 62.8° C. | Measured in |
| Calorimetry (DSC) | Tm₂ = 77.9° C. | Tm₂ = 68.0° C. | Tm₂ = 75.5° C. | 10 mM sodium |
| | Tm₃ = 84.0° C. | Tm₃ = 76.9° C. | Tm₃ = 83.3° C. | acetate pH 5.5 |
| | | Tm₄ = 83.3° C. | | |

7.3.4 Hydrophobicity of 05H21A, 15N14A, and 08F17A by aHIC

Relative hydrophobicity of 05H21A, 15N14A, and 08F17A was evaluated by hydrophobic interaction chromatography (HIC). Briefly, samples were diluted 1:5 in high salt buffer (100 mM Sodium Phosphate, 1.5M (NH₄)₂SO₄, pH 6.5) and approximately bugs of each sample was injected on a TOSOH TSKgel Butyl-NPR column on an Agilent HPLC instrument. HIC was run under a linear Amonium-SO4 gradient from 1.1M-0M over 8 minutes. UV280 and fluorescence (excitation at 280 nm and emission at 340 nm) signals were collected. The relative hydrophobicity was evaluated as retention time relative to internal hydrophobic standard (CNTO607) and reported as a hydrophobicity index (HI=rtAb/rtCNTO607). Retention times and hydrophobicity index of IL-1β antibodies are shown in Table 15.

The relative hydrophobicity was low for 05H21A and 15N14A (HI 0.37 and 0.49, respectively) but moderate for 08F17A (HI 0.80).

TABLE 15
Relative hydrophobicity by a HIC

| mAb Characteristic | 05H21A | 15N14A | 08F17A | CNTO607 |
|---|---|---|---|---|
| Relative | Low surface | Low surface | Moderate surface | High surface |
| Hydrophobicity | hydrophobicity: | hydrophobicity: | hydrophobicity: | hydrophobicity |
| (aHIC) | HI = 0.37 | HI = 0.49 | HI = 0.80 | HI = 1 |
| | Rt = 1.74 min | Rt = 2.32 min | Rt = 3.77 | rt = 4.72 min |

### 7.4 Example 4: Protein Production and Purification

7.4.1 Preparation of 05H21A, 15N14A and 08F17A

05H21A, 15N14A and 08F17A were expressed in stably transfected CHO cells and purified using column chromatography.

7.4.2 Protein Expression & Cell Culture

05H21A, 15N14A and 08F17A were expressed in a stably transfected Horizon CHO non-clonal pool which was generated by electroporation with purified plasmid DNA encoding to 05H21A, 15N14A or 08F17A (Heavy Chain and Light Chain) and Leap-In transposase mRNA (ATUM, Cat #LPN-1R) using a MaxCyte STx electroporator. After electroporation, standard expression protocols were followed for the generation of fed-batch. Each fed-batch was harvested and clarified by centrifugation followed by filtration.

7.4.3 Protein Purification

The filtered cell culture supernatants were purified using standard purification protocols. Briefly, each supernatant was loaded onto a pre-equilibrated Protein A column (GE Healthcare) using an AKTA chromatography system. After loading, the column was washed with 7 column volumes of 1×DPBS, pH 7.2. The protein was eluted with 2.5 column volumes of 0.1 M sodium acetate, pH 3.5. Protein fractions were neutralized immediately by the addition of 2.5 M Tris HCl, pH 7.5 to 8% (v/v) of the elution fraction volume and peak fractions were pooled.

The post-ProA elution pool was further purified by cation exchange chromatography (CEX) using Capto S Impact resin (GE Healthcare). The protein was eluted from the column with an increasing NaCl gradient in 20 mM MES pH 6.5. Only the peak fractions containing monomeric protein were pooled. The post-CEX elution pool was dialyzed into 10 mM sodium acetate pH 5.5 using a Pierce dialysis cassette (ThermoFisher) then filtered (0.2 μm).

7.4.4 Quality Control

The concentration of purified protein was determined by absorbance at 280 nm on a Dropsense spectrophotometer using the calculated extinction coefficient (Table 16). The quality of the purified protein was assessed by SDS-PAGE and analytical size exclusion HPLC (Agilent HPLC system). The endotoxin level was measured using a turbidimetric LAL assay (Pyrotell®-T, Associates of Cape Cod; Falmouth, Mass.). The QC data summary is found in Table 16.

TABLE 16
Protein Release Data Summary

| | | | | | | | Measured | | |
|---|---|---|---|---|---|---|---|---|---|
| GDB | Protein | Concen- | | Total | Monomer | Endo- | Mass | | |
| Batch | Descrip- | tration | Volume | Yield | % (SE- | toxin | G0F/G0F | Error | |
| ID# | tion | (mg/mL) | (mL) | (mg) | HPLC) | (Eu/mg) | [Da] | [ppm] | Buffer |
| 05H21A | 05H21A | 19.33 | 109 | 2107 | >99 | <1 | 147216.4 | 51.8 | 10 mM |
| | (anti-IL- | | | | | | | | sodium |
| | 1β) | | | | | | | | acetate |
| | mAb | | | | | | | | pH 5.5 |
| | [IgG1: | | | | | | | | |
| | YTE] | | | | | | | | |
| 15N14A | 15N14A | 15.06 | 79.0 | 1190 | 97.1 | <1 | 147842.9 | 39.0 | 10 mM |
| | (anti-IL- | | | | | | | | acetate |
| | 1β) | | | | | | | | pH 5.5 |
| | mAb | | | | | | | | |
| | [IgG1: | | | | | | | | |
| | YTE] | | | | | | | | |
| 08F17A | 08F17A | 18.12 | 58.9 | 1067 | 97.3 | <1 | 147368.5 | 26.3 | 10 mM |
| | (anti-IL- | | | | | | | | acetate |
| | 1β) | | | | | | | | pH 5.5 |
| | mAb | | | | | | | | |
| | [IgG1: | | | | | | | | |
| | YTE] | | | | | | | | |

### 7.5 Example 5: Functional Assays for Anti-IL-1β Antibodies

7.5.1 Experimental Methods

7.5.1.1 Inhibition of IL-β Signaling in HEK-Blue IL-1β Reporter Line

IL-1β HEK-Blue cells (cultured in DMEM with 10% heat-inactivated fetal bovine serum (FBS), 1% penicillin/streptomycin and 100 μg/ml Normocin) were collected and washed twice in PBS. Cells were resuspended in medium to a concentration of 330,000 cells/mL and 150 μl plated per well according to plate layout. Final cell count is 50,000 per well.

Test antibodies were diluted to a starting concentration of 40 nM (final concentration of 5 nM) and tested by serially diluting three-fold in medium. Recombinant human IL-1β (rhIL-1β) was diluted to 0.8 ng/mL (final concentration of 0.1 ng/mL or 6 pM) in medium. 100 μl of recombinant human IL-1β (rhIL-1β) and anti-IL-1β lead antibodies were co-incubated for 30 minutes at 37° C. in 5% CO₂. 50 μl of rhIL-1β/antibody complex was then added to cells and biological replicates were set up. The complex was then incubated with the cells for 18-20 hours at 37° C. with 5% CO₂. Following incubation, QuantiBlue solution was prepared and 180 μl added to a 96-well non-binding plate. 20 μl from each cell well was added to QuantiBlue solution and incubated for 30 minutes at 37° C. with 5% CO₂. Plates were then read at an optical density of 655 nm and anti-IL-1β lead antibody half-maximal inhibitory concentrations (IC50s) were calculated using the log (inhibitor) vs. response (variable slope) function in GraphPad.

7.5.1.2 Inhibition of IL-1β-induced IL-6 Production in MRCS Cells

IL-6 production in MRCS lung fibroblasts was assessed as previously described (Goh A X et al, MAbs. 2014; 6(3):765-773). MRCS fibroblast cells [ATCC #CCL-171] (cultured in EMEM with 10% heat-inactivated fetal bovine serum (FBS) and 1% penicillin/streptomycin) were trypsinized and counted. Cells were resuspended in medium to a concentration of 30,000 cells/mL and plated 3000 cells per well in a 96-well plate. Cells were allowed to recover for 16 hr overnight at 37° C./5% CO2. Test antibodies were diluted to a starting concentration of 10 nM and serially diluted in duplicate (in complete medium) in a 96-well plate using three-fold steps. 100 μL of each antibody dilution was transferred to a fresh 96-well plate. Recombinant IL-1β protein was diluted to 110 pg/mL in medium, and 11 μL added to each antibody well (final IL-1β concentration of 11 pg/mL; this concentration was previously determined as the EC50 for 3000 cells at 4 hr using IL-6 release as a readout). rhIL-1β and anti-IL-1β antibody serial dilutions were co-incubated for 30 minutes at 37° C. with 5% CO2. 100 μL of co-incubated complex was then added to cells according to plate layout. Cells were incubated for 4 hours at 37° C. with 5% CO2. 80 μL of media was then removed and transferred to a fresh 96 well plate and frozen at −80° C.

Media samples were thawed on ice and evaluated by ELISA. High binding ELISA plates were prepared by adding capture antibody to each well and incubating overnight as per product protocol (R&D IL-6 Duoset ELISA kit; Cat. No. DY206-05). 50 μL of collected cell media was assessed using this kit, with all steps following manufacturer's protocol. The kit provided recombinant hIL-6 as a positive control. The ELISA was developed using SureBlue TMB (KPL; #52-00-03) for 5 minutes at room temperature. The reaction was halted (TMB Stop Solution) and absorbances measured for 450 nm and 540 nm. Analysis was performed in GraphPad Prism. For each well, OD540 was subtracted from OD450 to normalize for optical imperfections of each well. The bottom row of each plate (diluent only) was averaged and subtracted from each sample to account for plate background. The IL-6 standard curve was used to calculate IL-6 in each sample. IC50 for each antibody was determined using a four-parameter variable slope analysis (log(agonist) vs. response) in GraphPad.

7.5.1.3 Inhibition of IL-1β-induced IL-6, ENA-78 (CXCL5) and G-CSF Production in NHDF

Donor human dermal fibroblasts (Lonza, Cat. No. CC-2509; Donors 29073 and 29114) were diluted to 5,000 cells/ml (final 1,000 cells/well in FGM-2 media) and 200 μl were added each well of a 96 well flat-bottom plates and incubated overnight at 37° C., 5% CO2. Test antibodies were prepared at a 3-fold dose titration of 4× antibodies with final concentrations from 10 nM-0.001 nM in FGM-2.

A 4× rhIL-1β was prepared (final assay concentration 10 pM rhIL-1β) by adding 41.4 μl of a 1:1000 dilution of rhIL-1β+49.959ml FGM-2. 200 μl of the 4× antibody titration and 200 μl of 4× rhIL-1β were combined and incubated at room temperature for 1 hr. Plated fibroblasts were washed with 200 μl of FBM media. Excess FBM was removed by flicking the plates and 100 μl of FGM-2 media was added per well. 100 μl of 2× antibody/rhIL-β mix was then added and the plates were incubated at 37° C., 5% CO2 for 18-20 hours. A total of 125 μl of supernatant was removed and transferred to a new 96-well round-bottom plate and stored at −20° C. for downstream MSD readout.

MSD U-Plex (Lonza, Cat. No. K15067L-4) was used to quantitate IL-6, ENA-78 (CXCL5), and G-CSF in test supernatants. Quantitation was performed according to manufacturer's alternative protocol, which adds detection antibodies directly to the sample with no wash in between. Standard curve fits were generated in Workbench using 4P linear regression curve fit with 1/(SD)\hat{ }2 weighting and percent inhibition (IC50) was calculated using GraphPad PRISM's 4P linear regression curve fit with no weighting [Percent Inhibition=100*(Pos Cntrl−Neg Cntrl)−(Sample−Neg Cntrl))/(Pos Cntrl−Neg. Cntrl)]. For any data point below the LLOD (Lower Limit of Detection), the value of the LLOD for the analyte calculated from the standard curve was substituted. For any data point above the ULOD (Upper Limit of Detection), the value was excluded.

7.5.1.4 Inhibition of IL-1β-Induced IL-6, ENA-78 (CXCL5) and G-CSF Production in NHLF

Donor human lung fibroblasts (Lonza, Cat. No. CC-2512; Donors 34325 and 35234) were diluted to 10,000 cells/ml (final 2,000 cells/well in FGM-2 media) and 200 μl were added each well of a 96 well flat-bottom plates and incubated overnight at 37° C., 5% CO2. Test antibodies were prepared at a 3-fold dose titration of 4× antibodies with final concentrations from 10 nM-0.001 nM in FGM-2 (test antibody ID and batch concentrations listed on table below).

A 4× rhIL-1β was prepared (final assay concentration 10 pM rhIL-1β) by adding 24.8 μl of a 1:1000 dilution of rhIL-1β+29.975ml FGM-2. 350 μl of the 4× antibody titration and 350 μl of 4× rhIL-1β were combined and incubated at room temperature for 1 hr. Plated fibroblasts were washed with 200 μl of FBM media. Excess FBM was removed by flicking the plates and 100 μl of FGM-2 media was added per well. 100 μl of 2× antibody/rhIL-β mix was then added and the plates were incubated at 37° C., 5% CO2 for 18-20 hrs. A total of 125 μl of supernatant was removed and transferred to a new 96-well round-bottom plate and stored at −20° C. for downstream MSD readout.

MSD U-Plex (Lonza, Cat. No. K15067L-4) was used to quantitate IL-6, ENA-78 (CXCL5), and G-CSF according to manufacturer's alternative protocol, which adds detection antibodies directly to sample with no wash in between. Standard curve fits were generated in Workbench using 4P linear regression curve fit with 1/(SD)\hat{ }2 weighting and percent inhibition (IC50) was calculated using GraphPad PRISM's 4P linear regression curve fit with no weighting [Percent Inhibition=100*(Pos Cntrl−Neg Cntrl)−(Sample−Neg Cntrl))/(Pos Cntrl−Neg. Cntrl)]. For any data point below the LLOD (Lower Limit of Detection), the value of the LLOD for the analyte calculated from the standard curve was substituted. For any data point above the ULOD (Upper Limit of Detection), the value was excluded.

7.5.1.5 Determination of IL-1β Induction Curve in human PBMCs

Treatment of PBMCs (peripheral blood mononuclear cells) with rhIL-1β induces release of IL-6 into the supernatant. A titration of IL-1β against human PBMCs was tested to determine and set EC50 and EC90 values for the subsequent evaluation of test antibodies. Blood from healthy donors was obtained and PBMCs were isolated by Ficoll-Hypaque using standard methods and frozen until use. PBMCs were thawed into prewarmed RPMI complete (RPMI containing 10% heat inactivated (HI), 1% penicillin, 1% streptomycin and 1% glutamine) and washed twice. Cells were counted and resuspended at 1 million per ml. 100 μl of cells were added to flat-bottomed, TC treated plate. rhIL-1β (201-LB/CF, R&D Systems) was serially diluted starting at a concentration 3× higher than the desired starting concentration and serial dilutions performed. 50 μl of dilution was added in duplicate or triplicate to the cells and the plates incubated 18-20 hours at 37° C. with 5% CO2.

IL-6 was measured in the supernatant using a human IL-6 ELISA kit (DY206, R&D Systems, adapted to half-area ELISA plates, Corning 3690). 50 μl of supernatant from each biological replicate was tested per well and the ELISA performed according to the manufacturer's instructions. The data were graphed and analyzed using Graphpad Prism and/or modeled values were determined by a Biostatistician.

TABLE 17
Modeled IL-1β EC50 and EC90
induction values across donors tested

| Donor | EC50 | | EC90 | |
|---|---|---|---|---|
| AC5684 | 4.419 | (3.231, 6.042) | 12.445 | (8.443, 18.344) |
| TS078.5 | 18.673 | (13.656, 25.532) | 71.474 | (48.49, 105.351) |
| TS083 | 17.935 | (13.117, 24.524) | 69.250 | (46.982, 102.073) |
| TS235 | 8.335 | (6.096, 11.397) | 29.074 | (19.725, 42.855) |
| TS274 | 9.418 | (6.888, 12.877) | 22.148 | (15.026, 32.645) |
| TS294 | 11.762 | (8.602, 16.082) | 43.364 | (29.42, 63.918) |
| TS301 | 7.347 | (5.373, 10.046) | 25.794 | (17.5, 38.02) |
| TS319 | 6.056 | (4.429, 8.281) | 26.520 | (17.992, 39.09) |
| TS320 | 8.472 | (6.196, 11.584) | 34.077 | (23.119, 50.229) |
| Model | 9.289 | (6.794, 12.702) | 32.468 | (22.028, 47.857) |

| | | |
|---|---|---|
| Median | 8.472 | 29.074 |
| Mean | 10.268 | 37.127 |
| Donor Range | [4.419, 18.673] | [12.445, 71.474] |

7.5.1.6 Inhibition of IL-1β-induced IL-6 Production in human PBMCs

Test antibodies were tested against 10 pM or 30 pM concentration of rhIL-1β (as determined in section 7.5.1.5) and IL-6 production was quantitated by ELISA in human PBMCs. Test antibodies were prepared at a 3× concentration (5 nM) and serially diluted 3-fold and mixed with an equal volume of rhIL-1β at the predetermined 10 pM (˜EC50) and 30 pM (˜EC90) concentrations. After a 60-minute incubation at 37° C. with 5% CO2, 100 μl of each mixture was added to a flat bottom TC treated plate. Controls included media alone to establish the baseline response, rhIL-1β (to measure the maximum response), and antibodies alone to ensure there was no IL-6 induction by the antibodies alone. During this incubation period, human PBMCs were prepared as above and brought to a concentration of 2 million per ml and 50 μl was added to each well. The cells were incubated for 18-20 hours at 37° C. with 5% CO2. IL-6 (Cat. No. DY206, R&D Systems, adapted to half-area ELISA plates, Corning3690) was measured using 50 μl of supernatant per well. Data were graphed and analyzed using GraphPad Prism to determine IC50s.

7.5.1.7 Inhibition of IL-1β-induced IL-6, ENA-78 (CXCL5) and G-CSF Production in Human Whole Blood Assays

Donor human whole blood was collected in ACD tubes and couriered to test site for immediate use. Test anti-IL-1β antibodies were serially diluted to a 10× concentration in 96-well U bottom plates in PBS containing 1% BSA, with the top final concentration at 10 nM and “no antibody” control included in the dilution series. rhIL-1β (R&D Systems) was prepared at 1000 pM in PBS containing 1% BSA and a final assay concentration of 100 pM was used. 10× antibody dilutions and 10× rhIL-1β dilution were combined at 1:1 and incubated at room temperature for 60 min. Human donor blood samples were mixed gently and 80 μl of whole blood was plated per well in 96 well U-bottom tissue culture plates. 20 μl of antibody+rhIL-1β mix was added per well and 1% PBS/BSA was added to control wells without rhIL-1β and/or antibody. A rhIL-1β dilution series was also prepared for confirmation of IL-1β dose response across patient samples. Treated cells were then incubated for 22±2 hrs at 37° C. After overnight incubation, 100 μl of PBS was added to all wells, mixed, and spun down in 96-well assay plates at 300×G for 10 minutes at room temperature. 100 μl of supernatant was transferred to a new 96-well plate, and a 25 μl of assay supernatant was used directly in MSD assay and analysis of IL-6, G-CSF and CXCL5 (ENA-78) was carried out according to manufacturer's instructions with the following exceptions: (i) concentration of calibrator #1 (IL-6) was doubled by diluting 2 vials in 125 μl each and combining them together, and (ii) an 11pt standard curve (4-fold dilution as instructed)+blank for 12 points in total was performed. Unknowns were calculated in MSD Workbench software and GraphPad prism was used to calculate EC values (rhIL-1β stimulation) and IC values (antibody inhibition) using 4P linear regression curve fit.

7.5.1.8 Establishing Species Relevancy: Inhibition of IL-1β Induced IL-6 and G-CSF Production in Cynomolgus Lung and Dermal Fibroblasts

Cynomolgus dermal and lung fibroblasts were diluted to 15,000 cells/ml (final 3,000 cells/well) in CFM media (Complete Fibroblast Media, Cell Biologics, Cat. No. M32267) and 200 μl were added to individual wells of a 96 well flat-bottom plates. Plate was incubated at 37° C., 5% CO2 overnight. A 3-fold dose titration of 4× test antibody was prepared with final concentrations from 10 nM-0.001 nM except for 08F17A, which had a dose range from 100 nM-0.01 nM in CFM. A 4× rcyno-IL-β (final assay concentration of 5 pM and 15 pM rcyno-IL-β for cyno dermal fibroblasts and cyno lung fibroblasts, respectively) was prepared in CFM and 200 μl of 4× rcynoIL-1β was combined with 200 μl of 4× test antibody titration, followed by incubation at RT for 1 hr. Plated dermal and lung fibroblasts were washed with 200 μl of FBM media and 100 μl of CFM media per well was added. 100 μl of 2× antibody/rcynoIL-β mix was then added to corresponding test well and incubated at 37° C., 5% CO2 for 18-20 hours. A total of 125 μl of supernatant was removed and transferred to a new 96-well round-bottom plate and stored at −20° C. for downstream MSD readout.

MSD U-Plex (Lonza, Cat. No. K15067L-4) was used to quantitate IL-6 and G-CSF according to manufacturer's alternative protocol, which adds detection antibodies directly to sample with no wash in between. Standard curve fits were generated in Workbench using 4P linear regression curve fit with 1/(SD)\hat{ }2 weighting and percent inhibition (IC50) was calculated using GraphPad PRISM's 4P linear regression curve fit with no weighting [Percent Inhibition=100*(Pos Cntrl−Neg Cntrl)−(Sample−Neg Cntrl))/(Pos Cntrl−Neg. Cntrl)]. For any data point below the LLOD (Lower Limit of Detection), the value of the LLOD for the analyte calculated from the standard curve was substituted. For any data point above the ULOD (Upper Limit of Detection), the value was excluded.

### Results

7.5.2 Anti-IL-1β mAbs Inhibit IL-1 Signaling in a Reporter Cell Line

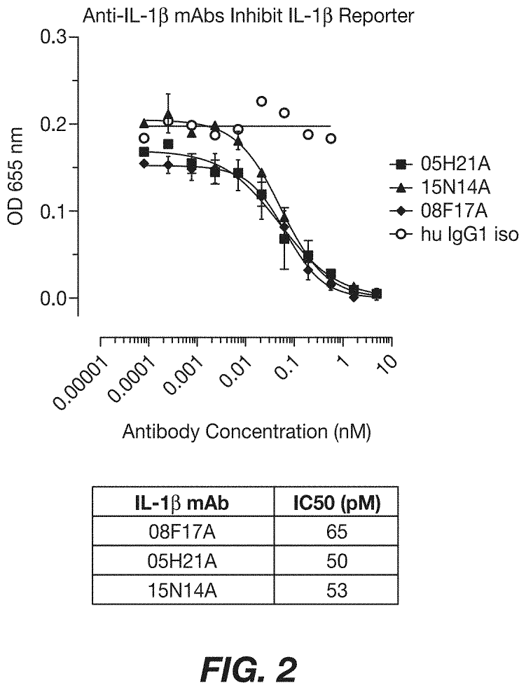

The ability of the anti-IL-1β lead panel to inhibit the IL-1 pathway was assessed in the HEK-Blue™ IL-1β reporter cell line (Invivogen). This reporter cell line has been engineered from the human embryonic kidney HEK 293 cells to detect bioactive IL-1β via the activation of an NF-κB and AP-1 inducible secreted embryonic alkaline phosphatase (SEAP) reporter. The cell line is responsive to both human IL-1β and IL-la as they signal via the same receptor, IL-1RI. In addition, the genes encoding for TNFR1, TLR3, and TLR5, which signal through NF-kB and AP-1 pathways, have been knocked out in this reporter line to avoid interference. In this study, 05H21A, 15N14A and 08F17A were assessed for neutralizing activity of the gene reporter. All mAbs exhibited a dose-dependent inhibition (FIG. 2). The IC50 values of all lead mAbs are shown in FIG. 2.

7.5.3 Anti-IL-1β mAbs Inhibit IL-1β-Induced IL-6 Production in a Lung Fibroblast Cell Line

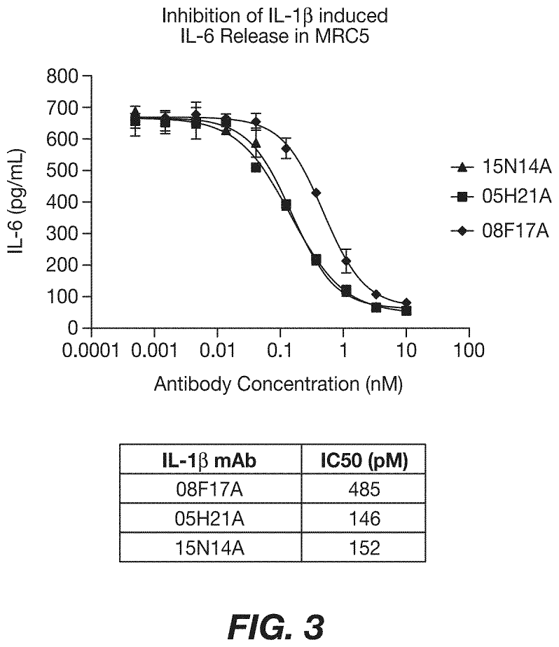

To further assess the neutralizing activity of the anti-IL-1β lead mAbs, an IL-6 release-based assay was set up in MRCS lung fibroblasts. IL-6 is a pleiotropic inflammatory cytokine that is primarily regulated by the IL-1 pathway via activation of the NF-kB and AP-1. As such, IL-1β activity can be readily measured through the production of IL-6 upon exposure of MRCS cells to rhIL-1β. 15N14A, 08F17A and 05H21A inhibited IL-6 release in a dose-dependent manner (FIG. 3). IC50 potency values for the three mAbs tested are reported in FIG. 3.

7.5.4 Anti-IL-1β mAbs Inhibit IL-1β Bioactivity in Primary Human Fibroblasts

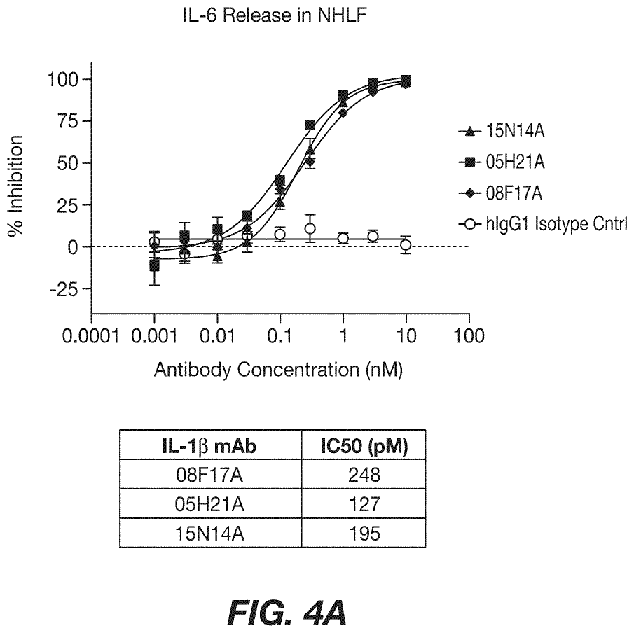

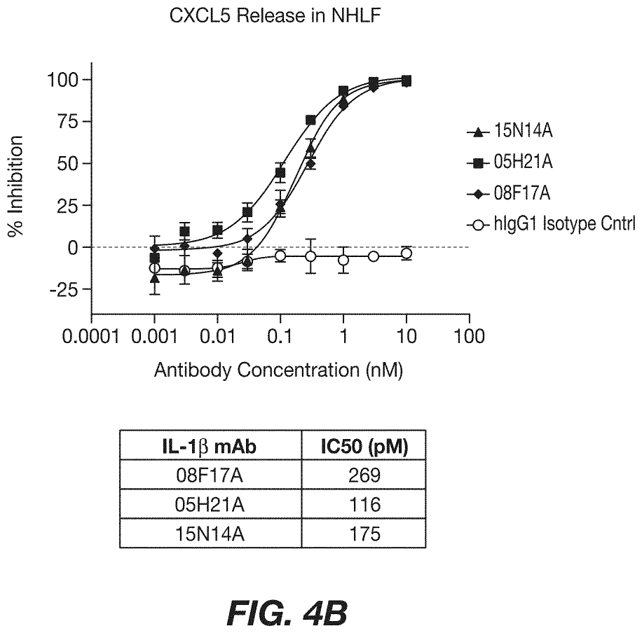

The inhibitory activity of 05H21A, 15N14A and 08F17A was assessed in primary human lung fibroblasts (donors #34325 and #35234). As a measure of IL-1β pathway activity, IL-6, ENA-78 (CXCL5) and G-CSF cytokine production and secretion was quantitated from culture supernatants via MSD. A final amount of 10 pM of rhIL-1β was used to ensure a full activation of the IL-1 pathway in these donor samples. All test mAbs exhibited a concentration-dependent neutralizing activity, which was based on IL-6 and CXCL5 (ENA-78) release measurements (FIG. 4A and FIG. 4B, respectively). IC50 potency values for the three mAbs tested are captured on FIGS. 4A and 4B. Assessment in primary dermal human fibroblasts (donor #29114) showed similar potencies of as in human lung fibroblast samples, with modeled IC30, IC50 and IC90 values reported in Table 18 in comparison to modeled IC30, IC50 and IC90 values determined in human lung fibroblast samples.

TABLE 18
Inhibitory values for IL-1β lead antibody panel in human lung and dermal fibroblasts

| Cell | Compound | Donors | IC30 (95% CI) | | IC50 (95% CI) | | IC90 (95% CI) | |
|---|---|---|---|---|---|---|---|---|
| NHLF | 05H21A | 34325 | 0.071 | (0.046, 0.11) | 0.167 | (0.11, 0.254) | 1.510 | (0.893, 2.555) |
| | | 35234 | 0.042 | (0.027, 0.065) | 0.098 | (0.065, 0.149) | 0.890 | (0.526, 1.505) |
| | 15N14A | 34325 | 0.128 | (0.095, 0.173) | 0.253 | (0.189, 0.339) | 1.479 | (1, 2.186) |
| | | 35234 | 0.090 | (0.067, 0.122) | 0.178 | (0.133, 0.239) | 1.043 | (0.705, 1.542) |
| | 08F17A | 34325 | 0.127 | (0.093, 0.175) | 0.293 | (0.216, 0.398) | 2.552 | (1.613, 4.038) |
| | | 35234 | 0.090 | (0.065, 0.123) | 0.207 | (0.152, 0.281) | 1.803 | (1.139, 2.852) |
| NHDF | 05H21A | 29114 | 0.039 | (0.030, 0.050) | 0.078 | (0.065, 0.095) | 0.490 | (0.323, 0.743) |
| | 15N14A | | 0.069 | (0.049, 0.095) | 0.127 | (0.098, 0.165) | 0.626 | (0.349, 1.123) |
| | 08F17A | | 0.087 | (0.061, 0.124) | 0.174 | (0.130, 0.234) | 1.073 | (0.541, 2.129) |

Modeled IC30, IC50 and IC90 values in human dermal fibroblast (NHDF donor # 29114) and human lung fibroblasts (NHLF donors #34325 and #35234) are reported in nM and CI indicates confidence interval.

7.5.5 mAbs Inhibit IL-1β Bioactivity in Human Donor PBMC Samples

Induction experiments using rhIL-1β were performed across a panel of healthy human PBMC donors. Modeled EC50 and EC90 induction estimates were determined based on rhIL-1β-induced IL-6 cytokine release measurements using a nonlinear mixed effects model (Table 17). The rhIL-1β EC50 induction values across donors ranged from 4.42 to 18.67 pM with a median value of 8.4 pM; and the rhIL-1β EC90 values ranged from 12.45 to 71.47 pM with a median value of 29.1 pM. From these studies, 10 pM and 30 pM of rhIL-1β were selected as representative EC50 and EC90 values to be used in subsequent potency studies in donor PBMC assays.

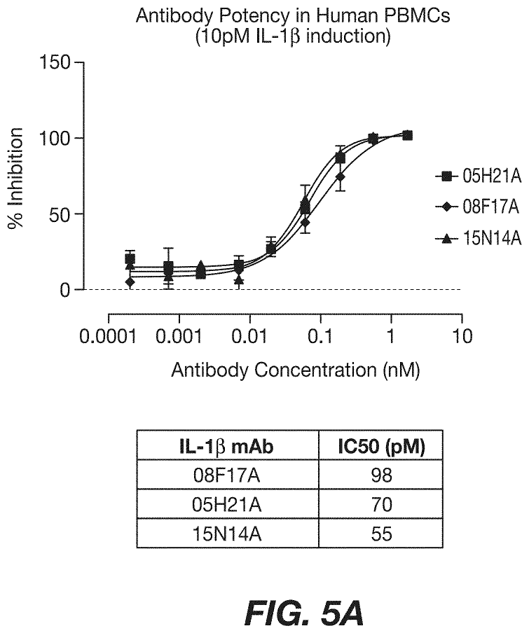

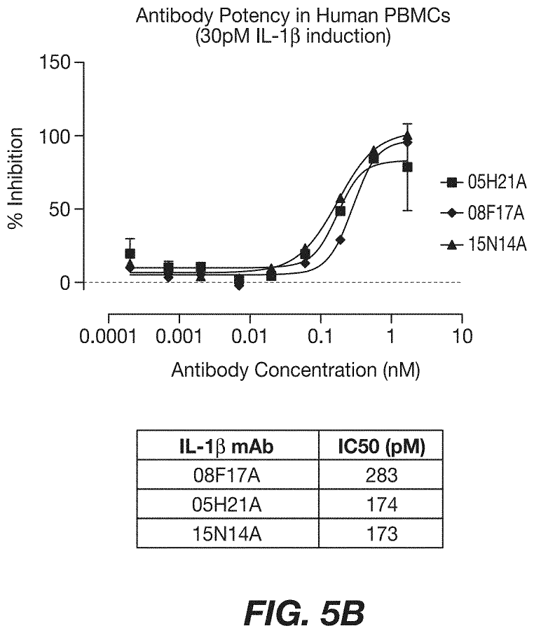

The functional activity of the anti-IL-1β mAbs, 15N14A, 05H21A and 08F17A, was then evaluated in a panel of five PBMC donors using the two predetermined rhIL-1β induction concentrations. All anti-IL-1β mAbs exhibited a dose-dependent inhibition as shown in a representative donor PBMC sample (FIGS. 5A and 5B). Modeled IC30, IC50 and IC90 estimated means (n=5 donor PBMCs) are reported in Table 19.

TABLE 19
Inhibitory values for IL-1β lead antibody panel in human PBMC samples

| Treatment | IC | 05H21A | 08F17A | 15N14A |
|---|---|---|---|---|
| 10 pM IL1β | 30 | 0.043 (0.028, 0.067) | 0.052 (0.034, 0.082) | 0.032 (0.018, 0.059) |
| | 50 | 0.070 (0.045, 0.107) | 0.092 (0.060, 0.142) | 0.056 (0.031, 0.099) |
| | 90 | 0.244 (0.146, 0.407) | 0.402 (0.212, 0.765) | 0.226 (0.108, 0.474) |
| 30 pM IL1β | 30 | 0.104 (0.075, 0.144) | 0.156 (0.115, 0.212) | 0.075 (0.056, 0.099) |
| | 50 | 0.164 (0.120, 0.223) | 0.241 (0.178, 0.328) | 0.120 (0.092, 0.155) |
| | 90 | 0.533 (0.294, 0.969) | 0.744 (0.487, 1.136) | 0.409 (0.228, 0.734) |

*Modeled IC30, IC50 and IC90 estimated means based on dose-response analysis of percent inhibition from a representative IL-6 release assay conducted in five PBMC donor samples. Values are reported in nM.

7.5.6 Anti-1β mAbs Inhibit IL-1β Bioactivity in Human Whole Blood Samples

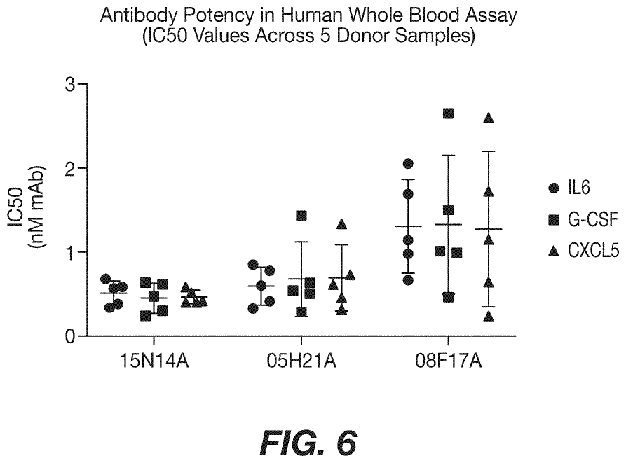

As a final measure of potency, the neutralizing activity the anti-IL-1β mAb panel was assessed in human whole blood donor samples. Potency assessment in whole blood samples provides a more physiologically relevant setting compared to assessment in PBMC samples given the presence of serum proteins. The potency of 15N14A, 08F17A and 05H21A was assessed in 5 donor samples and was based on IL-6, CXCL-5 and G-CSF cytokine release measurements (FIG. 6). Modeled IC30, IC50 and IC90 estimated means from 5 blood donor samples were based on dose-response analysis of percent inhibition and are reported on Table 20.

TABLE 20
Inhibitory values for IL-1β lead antibody panel in human whole blood assay

| Cytokine | Compound | IC30 (95% CI) | IC50 (95% CI) | | IC90 (95% CI) | |
|---|---|---|---|---|---|---|
| IL-6 | 15N14A | 0.360 (0.263, 0.493) | 0.595 | (0.450, 0.786) | 2.183 | (1.200, 3.974) |
| | 05H21A | 0.393 (0.280, 0.552) | 0.631 | (0.465, 0.857) | 2.156 | (1.064, 4.368) |
| | 08F17A | 0.872 (0.513, 1.483) | 1.529 | (0.853, 2.743) | 6.559 | (1.895, 22.703) |
| G-CSF | 15N14A | 0.299 (0.183, 0.488) | 0.520 | (0.329, 0.820) | 2.179 | (0.813, 5.840) |
| | 05H21A | 0.364 (0.221, 0.600) | 0.632 | (0.394, 1.016) | 2.643 | (1.026, 6.807) |
| | 08F17A− | 0.704 (0.398, 1.246) | 1.311 | (0.683, 2.516) | 6.576 | (1.549, 27.909) |
| CXCL5 | 15N14A | 0.364 (0.244, 0.542) | 0.603 | (0.420, 0.867) | 2.236 | (0.981, 5.098) |
| | 05H21A | 0.400 (0.249, 0.642) | 0.622 | (0.403, 0.961) | 1.953 | (0.757, 5.035) |
| | 08F17A | 1.330 (0.437, 4.049) | 2.859 | (0.646, 12.642) | 20.797 | (0.860, 502.943) |

* Modeled IC30, IC50 and IC90 estimated means based on dose-response analysis of percent inhibition from an IL-6, CXCL5, and G-CSF release assay conducted in five healthy human blood samples (donors CC00448, M3767, M5988, M7286, and M7370). Data is reported in nM and CI denotes confidence interval.

7.5.7 NHP Relevancy: Anti-IL-1β mAbs Inhibit IL-1β Bioactivity in Cynomolgus Monkey Fibroblasts

To enable toxicology studies in primates, the cross-reactivity of 15N14A, 08F17A and 05H21A were assessed in cynomolgus macaques. At the amino acid level, the mature form of human and cynomolgus macaque IL-1β are 95% homologous. Affinity determination via BIAcore indicated high-affinity to IL-1β from cynomolgus monkeys for the three lead mAbs, suggesting a conserved epitope in cynomolgus macaques (Table 12). In contrast, canakinumab does not cross-react with cynomolgus IL-1β as its antigenic epitope includes Glu64 in the human sequence, which is key for the recognition of the antibody absent in the cynomolgus IL-1β sequence. (Dhimolea E. Canakinumab. MAbs. 2010; 2(1):3-13). In fact, marmoset was identified as the only non-human primate species carrying Glu64, thus enabling toxicological studies for canakinumab in this species. (Rondeau J M, Ramage P, Zurini M, Gram H. The molecular mode of action and species specificity of canakinumab, a human monoclonal antibody neutralizing IL-1beta. MAbs. 2015; 7(6):1151-1160).

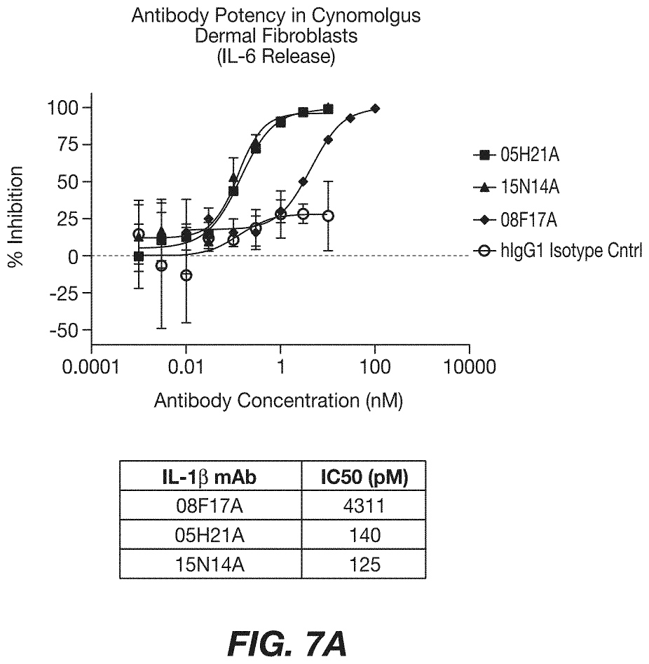

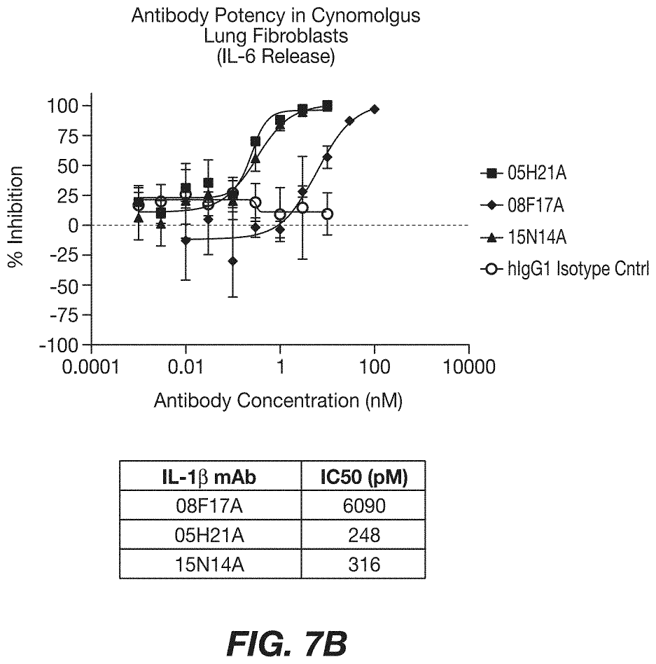

Given the high affinity to cynomolgus IL-1β and the epitope mapping data by deuterium exchange, we predicted that our lead panel would exhibit functional activity in a cynomolgus cell-based assays. To confirm our prediction, we established IL-6 and G-CSF release-based assays with cynomolgus recombinant IL-1β in both primary dermal and lung cynomolgus fibroblasts. The neutralizing activity of 05H21A, 15N14A and 08F17A in both cynomolgus primary fibroblasts was then evaluated. All lead anti-IL-1β mAbs exhibited a dose-dependent neutralizing activity in dermal and lung cynomolgus fibroblasts (FIGS. 7A and 7B). Cynomolgus dermal fibroblasts (CDF) and cynomolgus lung fibroblasts (CLF) were activated with 5 pM and 15 pM of recombinant cynomolgus IL-1β, respectively. Table 21 captures the modeled IC30, IC50 and IC90 estimates in cynomolgus dermal (CDF) and lung fibroblasts (CLF). The results shown here confirmed the functional activity of all three mAbs in cynomolgus macaques, thereby establishing the relevancy of this species to enable downstream toxicology studies.

TABLE 21
Inhibitory values for IL-1β lead antibody panel in primary cynomolgus fibroblasts

| | | | | |
|---|---|---|---|---|
| Dose | Compound | IC30 (95% CI) | IC50 (95% CI) | IC90 (95% CI) |

IL-6

| | | | | | | |
|---|---|---|---|---|---|---|
| 5 pM IL1β | 15N14A | 0.093 (0.051, 0.171) | 0.156 | (0.095, 0.257) | 0.594 | (0.193, 1.833) |
| (CDF) | 05H21A | 0.089 (0.055, 0.145) | 0.175 | (0.117, 0.260) | 0.997 | (0.396, 2.510) |
| | 08F17A | 2.722 (1.981, 3.741) | 5.096 | (3.766, 6.895) | 25.904 | (12.420, 54.030) |
| 15 pM IL1β | 15N14A | 0.192 (0.095, 0.391) | 0.376 | (0.197, 0.718) | 2.140 | (0.443, 10.335) |
| (CLF) | 05H21A | 0.214 (0.124, 0.367) | 0.306 | (0.207, 0.453) | 0.779 | (0.324, 1.874) |
| | 08F17A | 3.435 (1.585, 7.448) | 6.969 | (3.064, 15.851) | 43.624 | (5.649, 336.854) |

G-CSF

| | | | | | | |
|---|---|---|---|---|---|---|
| 5 pM IL1β | 05H21A | 0.046 (0.031, 0.069) | 0.059 | (0.037, 0.095) | 0.110 | (0.041, 0.299) |
| (CDF) | 08F17A | 1.923 (1.453, 2.544) | 3.236 | (2.555, 4.099) | 12.480 | (7.193, 21.651) |
| 15 pM IL1β | 15N14A | 0.129 (0.063, 0.264) | 0.261 | (0.140, 0.487) | 1.629 | (0.363, 7.302) |
| (CLF) | 05H21A | 0.195 (0.108, 0.353) | 0.270 | (0.174, 0.421) | 0.630 | (0.283, 1.400) |
| | 08F17A | 3.584 (1.519, 8.456) | 6.106 | (2.722, 13.695) | 24.308 | (3.671, 160.959) |

Model for 15N14A for 5 pM of IL1β (CDF) for cytokine G-CSF failed to converge.
* Modeled IC30, IC50 and IC90 estimates for IL-6 and G-CSF release measured after stimulation with 5 pM and 15 pM of rcynoIl-1β in cynomolgus dermal (CDF) and lung fibroblasts (CLF), respectively. Data is reported in nM and CI denotes confidence interval.

1. An antibody that binds IL-1β comprising:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 8;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 9; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 10; or
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 11; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 12.

2. The antibody of claim 1, (i) wherein the VH CDR1, VH CDR2, VH CDR3, VL CDR1, VL CDR2, and VL CDR3 amino acid sequences are according to the Kabat numbering system; (ii) wherein the VH CDR1, VH CDR2, VH CDR3, VL CDR1, VL CDR2, and VL CDR3 amino acid sequences are according to the Chothia numbering system; (iii) wherein the VH CDR1, VH CDR2, VH CDR3, VL CDR1, VL CDR2, and VL CDR3 amino acid sequences are according to the AbM numbering system; (iv) wherein the VH CDR1, VH CDR2, VH CDR3, VL CDR1, VL CDR2, and VL CDR3 amino acid sequences are according to the Contact numbering system; and/or (v) wherein the VH CDR1, VH CDR2, VH CDR3, VL CDR1, VL CDR2, and VL CDR3 amino acid sequences are according to the IMGT numbering system.

3. An antibody that binds IL-1β comprising:
(1) (i) a VH comprising a VH CDR1 having an amino acid sequence selected from SEQ ID NO: 13, SEQ ID NO: 31, SEQ ID NO: 49, SEQ ID NO: 67, and SEQ ID NO: 85; a VH CDR2 having an amino acid sequence selected from SEQ ID NO: 14, SEQ ID NO: 32, SEQ ID NO: 50, SEQ ID NO: 68, and SEQ ID NO: 86; a VH CDR3 having an amino acid sequence selected from SEQ ID NO: 15, SEQ ID NO: 33, SEQ ID NO: 51, SEQ ID NO: 69, and SEQ ID NO: 87; and
(ii) a VL comprising a VL CDR1 having an amino acid sequence selected from SEQ ID NO: 16, SEQ ID NO: 34, SEQ ID NO: 52, SEQ ID NO: 70, and SEQ ID NO: 88; a VL CDR2 having an amino acid sequence selected from SEQ ID NO: 17, SEQ ID NO: 35, SEQ ID NO: 53, SEQ ID NO: 71, and SEQ ID NO: 89; a VL CDR3 having an amino acid sequence selected from SEQ ID NO: 18, SEQ ID NO: 36, SEQ ID NO: 54, SEQ ID NO: 72, and SEQ ID NO: 90;
(2) (i) a VH comprising a VH CDR1 having an amino acid sequence selected from SEQ ID NO: 19, SEQ ID NO: 37, SEQ ID NO: 55, SEQ ID NO: 73, and SEQ ID NO: 91; a VH CDR2 having an amino acid sequence selected from SEQ ID NO: 20, SEQ ID NO: 38, SEQ ID NO: 56, SEQ ID NO: 74, and SEQ ID NO: 92; a VH CDR3 having an amino acid sequence selected from SEQ ID NO: 21, SEQ ID NO: 39, SEQ ID NO: 57, SEQ ID NO: 75, and SEQ ID NO: 93; and
(ii) a VL comprising a VL CDR1 having an amino acid sequence selected from SEQ ID NO: 22, SEQ ID NO: 40, SEQ ID NO: 58, SEQ ID NO: 76, and SEQ ID NO: 94; a VL CDR2 having an amino acid sequence selected from SEQ ID NO: 23, SEQ ID NO: 41, SEQ ID NO: 59, SEQ ID NO: 77, and SEQ ID NO: 95; a VL CDR3 having an amino acid sequence selected from SEQ ID NO: 24, SEQ ID NO: 42, SEQ ID NO: 60, SEQ ID NO: 78, and SEQ ID NO: 96; or
(3) (i) a VH comprising a VH CDR1 having an amino acid sequence selected from SEQ ID NO: 25, SEQ ID NO: 43, SEQ ID NO: 61, SEQ ID NO: 79, and SEQ ID NO: 97; a VH CDR2 having an amino acid sequence selected from SEQ ID NO: 26, SEQ ID NO: 44, SEQ ID NO: 62, SEQ ID NO: 80, and SEQ ID NO: 98; a VH CDR3 having an amino acid sequence selected from SEQ ID NO: 27, SEQ ID NO: 45, SEQ ID NO: 63, SEQ ID NO: 81, and SEQ ID NO: 99; and
(ii) a VL comprising a VL CDR1 having an amino acid sequence selected from SEQ ID NO: 28, SEQ ID NO: 46, SEQ ID NO: 64, SEQ ID NO: 82, and SEQ ID NO: 100; a VL CDR2 having an amino acid sequence selected from SEQ ID NO: 29, SEQ ID NO: 47, SEQ ID NO: 65, SEQ ID NO: 83, and SEQ ID NO: 101; a VL CDR3 having an amino acid sequence selected from SEQ ID NO: 30, SEQ ID NO: 48, SEQ ID NO: 66, SEQ ID NO: 84, and SEQ ID NO: 102.

4. An antibody that binds IL-1β comprising:
(1) (i) a VH comprising a VH CDR1 having an amino acid sequence of SEQ ID NO: 13; a VH CDR2 having an amino acid sequence of SEQ ID NO: 14; a VH CDR3 having an amino acid sequence of SEQ ID NO: 15; and
(ii) a VL comprising a VL CDR1 having an amino acid sequence of SEQ ID NO: 16; a VL CDR2 having an amino acid sequence selected of SEQ ID NO: 17; a VL CDR3 having an amino acid sequence of SEQ ID NO: 18;
(2) (i) a VH comprising a VH CDR1 having an amino acid sequence of SEQ ID NO: 19; a VH CDR2 having an amino acid sequence of SEQ ID NO: 20; a VH CDR3 having an amino acid sequence of SEQ ID NO: 21; and
(ii) a VL comprising a VL CDR1 having an amino acid sequence of SEQ ID NO: 22; a VL CDR2 having an amino acid sequence selected of SEQ ID NO: 23; a VL CDR3 having an amino acid sequence of SEQ ID NO: 24;
(3) (i) a VH comprising a VH CDR1 having an amino acid sequence of SEQ ID NO: 25; a VH CDR2 having an amino acid sequence of SEQ ID NO: 26; a VH CDR3 having an amino acid sequence of SEQ ID NO: 27; and
(ii) a VL comprising a VL CDR1 having an amino acid sequence of SEQ ID NO: 28; a VL CDR2 having an amino acid sequence selected of SEQ ID NO: 29; a VL CDR3 having an amino acid sequence of SEQ ID NO: 30;
(4) (i) a VH comprising a VH CDR1 having an amino acid sequence of SEQ ID NO: 31; a VH CDR2 having an amino acid sequence of SEQ ID NO: 32; a VH CDR3 having an amino acid sequence of SEQ ID NO: 33; and
(ii) a VL comprising a VL CDR1 having an amino acid sequence of SEQ ID NO: 34; a VL CDR2 having an amino acid sequence selected of SEQ ID NO: 35; a VL CDR3 having an amino acid sequence of SEQ ID NO: 36;
(5) (i) a VH comprising a VH CDR1 having an amino acid sequence of SEQ ID NO: 37; a VH CDR2 having an amino acid sequence of SEQ ID NO: 38; a VH CDR3 having an amino acid sequence of SEQ ID NO: 39; and
(ii) a VL comprising a VL CDR1 having an amino acid sequence of SEQ ID NO: 40; a VL CDR2 having an amino acid sequence selected of SEQ ID NO: 41; a VL CDR3 having an amino acid sequence of SEQ ID NO: 42;
(6) (i) a VH comprising a VH CDR1 having an amino acid sequence of SEQ ID NO: 43; a VH CDR2 having an amino acid sequence of SEQ ID NO: 44; a VH CDR3 having an amino acid sequence of SEQ ID NO: 45; and
(ii) a VL comprising a VL CDR1 having an amino acid sequence of SEQ ID NO: 46; a VL CDR2 having an amino acid sequence selected of SEQ ID NO: 47; a VL CDR3 having an amino acid sequence of SEQ ID NO: 48;
(7) (i) a VH comprising a VH CDR1 having an amino acid sequence of SEQ ID NO: 49; a VH CDR2 having an amino acid sequence of SEQ ID NO: 50; a VH CDR3 having an amino acid sequence of SEQ ID NO: 51; and
(ii) a VL comprising a VL CDR1 having an amino acid sequence of SEQ ID NO: 52; a VL CDR2 having an amino acid sequence selected of SEQ ID NO: 53; a VL CDR3 having an amino acid sequence of SEQ ID NO: 54;
(8) (i) a VH comprising a VH CDR1 having an amino acid sequence of SEQ ID NO: 55; a VH CDR2 having an amino acid sequence of SEQ ID NO: 56; a VH CDR3 having an amino acid sequence of SEQ ID NO: 57; and
(ii) a VL comprising a VL CDR1 having an amino acid sequence of SEQ ID NO: 58; a VL CDR2 having an amino acid sequence selected of SEQ ID NO: 59; a VL CDR3 having an amino acid sequence of SEQ ID NO: 60;
(9) (i) a VH comprising a VH CDR1 having an amino acid sequence of SEQ ID NO: 61; a VH CDR2 having an amino acid sequence of SEQ ID NO: 62; a VH CDR3 having an amino acid sequence of SEQ ID NO: 63; and
(ii) a VL comprising a VL CDR1 having an amino acid sequence of SEQ ID NO: 64; a VL CDR2 having an amino acid sequence selected of SEQ ID NO: 65; a VL CDR3 having an amino acid sequence of SEQ ID NO: 66;
(10) (i) a VH comprising a VH CDR1 having an amino acid sequence of SEQ ID NO: 67; a VH CDR2 having an amino acid sequence of SEQ ID NO: 68; a VH CDR3 having an amino acid sequence of SEQ ID NO: 69; and
(ii) a VL comprising a VL CDR1 having an amino acid sequence of SEQ ID NO: 70; a VL CDR2 having an amino acid sequence selected of SEQ ID NO: 71; a VL CDR3 having an amino acid sequence of SEQ ID NO: 72;
(11) (i) a VH comprising a VH CDR1 having an amino acid sequence of SEQ ID NO: 73; a VH CDR2 having an amino acid sequence of SEQ ID NO: 74; a VH CDR3 having an amino acid sequence of SEQ ID NO: 75; and
(ii) a VL comprising a VL CDR1 having an amino acid sequence of SEQ ID NO: 76; a VL CDR2 having an amino acid sequence selected of SEQ ID NO: 77; a VL CDR3 having an amino acid sequence of SEQ ID NO: 78;
(12) (i) a VH comprising a VH CDR1 having an amino acid sequence of SEQ ID NO: 79; a VH CDR2 having an amino acid sequence of SEQ ID NO: 80; a VH CDR3 having an amino acid sequence of SEQ ID NO: 81; and
(ii) a VL comprising a VL CDR1 having an amino acid sequence of SEQ ID NO: 82; a VL CDR2 having an amino acid sequence selected of SEQ ID NO: 83; a VL CDR3 having an amino acid sequence of SEQ ID NO: 84;
(13) (i) a VH comprising a VH CDR1 having an amino acid sequence of SEQ ID NO: 85; a VH CDR2 having an amino acid sequence of SEQ ID NO: 86; a VH CDR3 having an amino acid sequence of SEQ ID NO: 87; and
(ii) a VL comprising a VL CDR1 having an amino acid sequence of SEQ ID NO: 88; a VL CDR2 having an amino acid sequence selected of SEQ ID NO: 89; a VL CDR3 having an amino acid sequence of SEQ ID NO: 90;
(14) (i) a VH comprising a VH CDR1 having an amino acid sequence of SEQ ID NO: 91; a VH CDR2 having an amino acid sequence of SEQ ID NO: 92; a VH CDR3 having an amino acid sequence of SEQ ID NO: 93; and
(ii) a VL comprising a VL CDR1 having an amino acid sequence of SEQ ID NO: 94; a VL CDR2 having an amino acid sequence selected of SEQ ID NO: 95; a VL CDR3 having an amino acid sequence of SEQ ID NO: 96; or
(15) (i) a VH comprising a VH CDR1 having an amino acid sequence of SEQ ID NO: 97; a VH CDR2 having an amino acid sequence of SEQ ID NO: 98; a VH CDR3 having an amino acid sequence of SEQ ID NO: 99; and
(ii) a VL comprising a VL CDR1 having an amino acid sequence of SEQ ID NO: 100; a VL CDR2 having an amino acid sequence selected of SEQ ID NO: 101; a VL CDR3 having an amino acid sequence of SEQ ID NO: 102.

5. The antibody of claim 1, wherein the antibody further comprises one or more framework regions as set forth in SEQ ID NO: 7, SEQ ID NO: 8, SEQ ID NO: 9, SEQ ID NO: 10, SEQ ID NO: 11, and/or SEQ ID NO: 12.

6. The antibody of claim 1, wherein:
(i) the antibody comprises a VH having an amino acid sequence of SEQ ID NO: 7, and a VL having an amino acid sequence of SEQ ID NO: 8;
(ii) the antibody comprises a VH having an amino acid sequence of SEQ ID NO: 9, and a VL having an amino acid sequence of SEQ ID NO: 10; or
(iii) the antibody comprises a VH having an amino acid sequence of SEQ ID NO: 11, and a VL having an amino acid sequence of SEQ ID NO: 12.

7. The antibody of claim 1, wherein:
a) the antibody is a humanized antibody;
b) the antibody is an IgG antibody, wherein optionally the IgG antibody is an IgG1, IgG2, IgG3, or IgG4 antibody;
c) the antibody comprises a kappa light chain;
d) the antibody comprises a lambda light chain;
e) the antibody comprises a mutant Fc region, wherein optionally the mutant Fc region comprises M252Y/S254T/T256E (YTE) mutations;
f) the antibody is a monoclonal antibody;
g) the antibody binds an IL-1β antigen;
h) the antibody binds an IL-1β epitope;
i) the antibody specifically binds to IL-1β,
j) the VH CDR1, VH CDR2, VH CDR3, VL CDR1, VL CDR2 and VL CDR3 form a binding site for an antigen of the IL-1β, and/or
k) the VH CDR1, VH CDR2, VH CDR3, VL CDR1, VL CDR2 and VL CDR3 form a binding site for an epitope of the IL-1β.

8-19. (canceled)

20. The antibody of claim 1, wherein the antibody is multispecific, wherein optionally:
a) the antibody is capable of binding at least two antigens;
b) the antibody is capable of binding at least three antigens;
c) the antibody is capable of binding at least four antigens; or
d) the antibody is capable of binding at least five antigens.

21-24. (canceled)

25. A binding molecule comprising the antibody of claim 1, wherein the antibody is genetically fused or chemically conjugated to an agent.

26. A nucleic acid encoding the antibody of claim 1.

27. A vector comprising the nucleic acid of claim 26.

28. A host cell comprising the vector of claim 27.

29. A kit comprising the vector of claim 27 and packaging for the same.

30. A kit comprising the antibody of claim 1 and packaging for the same.

31. A pharmaceutical composition comprising the antibody of claim 1, and one or more pharmaceutically acceptable excipients.

32. A method of producing the pharmaceutical composition of claim 31, comprising combining the antibody with one or more pharmaceutically acceptable excipients to obtain the pharmaceutical composition.

33. A method of
a) inhibiting IL-1β or IL-1β mediated signaling in a cell, comprising the contacting the cell with the antibody of claim 1;
b) inhibiting IL-1β induced production of IL-6, ENA-78 (CXCL5) and/or G-CSF in a cell, comprising the contacting the cell with the antibody of claim 1;
c) decreasing the production of IL-6, ENA-78 (CXCL5) and/or G-CSF in a cell, comprising the contacting the cell with the antibody of claim 1; or
d) inhibiting growth or proliferation of IL-1β expressing cells, comprising contacting the cells with the antibody of claim 1,
wherein optionally the cell or the cells are in a subject having a disease or disorder.

34-37. (canceled)

38. A method of inhibiting IL-1β in a subject, comprising administering to the subject the antibody of claim 1.

39. A method for treating a disease or disorder in a subject, comprising administering to the subject the antibody of claim 1.

40. The method of claim 39, wherein the disease or disorder is an IL-1β associated disease or disorder, wherein optionally:
a) the IL-1β associated disease or disorder is an inflammatory disease or disorder; or
b) the IL-1β associated disease or disorder is cancer, wherein optionally:
i) the cancer is lung cancer, wherein optionally the lung cancer is non-small cell lung cancer, wherein optionally the non-small cell lung cancer has reached stage 0, stage 1, stage 2, stage 3, or stage 4; or
ii) the cancer is kidney cancer, wherein optionally the kidney cancer is renal cell cancer, wherein optionally the renal cell cancer has reached stage 1, stage 2, or stage 3.

41-47. (canceled)


SEQUENCE LISTING (110 sequences)

SEQ ID NO: 1 - protein, 450 aa, synthetic construct
  source 1..450: note = 05H21A HC
QVTLRESGPALVKPTQTLTLTCTFSGFSLSTSGMWVSWIRQPPGKALEWLALIDWGDDKYYTTSLKTRLTISKDTSKNQVVLTMTNMDPVDTATYYCARMREGSRAFDIWGQGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, synthetic construct
  source 1..216: note = 05H21A LC
QSVLTQPPSVSEAPRQRVTISCSGSSSNIGDNAVNWYQQLPGKAPKLLIYNDDLLSSGVSDRFSGSKSGTSASLAISGLQSEDEADYYCAAWDDSLNGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 449 aa, synthetic construct
  source 1..449: note = 15N14A HC
QVQLQESGPGLVKPSETLSLTCTVSGGSISSYYWTWIRQPAGKGLEWIGRIDSSGSSKYNPTLKSRVTMSVDTSKNQFSLKLSSVTAADTAVYYCARGDSGYDWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 221 aa, synthetic construct
  source 1..221: note = 15N14A LC
QAVLTQPSSLSASPGASASLTCTLRSGINVGTYRIYWYQQKPGSPPQYLLSYKSDSDKQQGSGVPSRFSGSKDASANVGILLISGLQSEDEADYYCMIWHSSAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 448 aa, synthetic construct
  source 1..448: note = 08F17A HC
EVQLVESGGGLVTPGGSLRLSCAASGFTFSGYSMNWVRQAPGKGLEWVSSISSSSGYIYYADSVKGRFTISRDNAKNSLYLQMNSLRAEDTAVYYCAREYWGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct
  source 1..214: note = 08F17A LC
DIQMTQSPSSLSASVGDRVTITCRASQGISYYLAWYQQKPGKVPKLLISAEFTLQSGVPSRFSGSGSGTDFTLTISSLQPEDVATYYCQKYNTAPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 120 aa, synthetic construct
  source 1..120: note = 05H21A VH
QVTLRESGPALVKPTQTLTLTCTFSGFSLSTSGMWVSWIRQPPGKALEWLALIDWGDDKYYTTSLKTRLTISKDTSKNQVVLTMTNMDPVDTATYYCARMREGSRAFDIWGQGTVVTVSS

SEQ ID NO: 8 - protein, 110 aa, synthetic construct
  source 1..110: note = 05H21A VL
QSVLTQPPSVSEAPRQRVTISCSGSSSNIGDNAVNWYQQLPGKAPKLLIYNDDLLSSGVSDRFSGSKSGTSASLAISGLQSEDEADYYCAAWDDSLNGPVFGGGTKLTVL

SEQ ID NO: 9 - protein, 119 aa, synthetic construct
  source 1..119: note = 15N14A VH
QVQLQESGPGLVKPSETLSLTCTVSGGSISSYYWTWIRQPAGKGLEWIGRIDSSGSSKYNPTLKSRVTMSVDTSKNQFSLKLSSVTAADTAVYYCARGDSGYDWAFDYWGQGTLVTVSS

SEQ ID NO: 10 - protein, 115 aa, synthetic construct
  source 1..115: note = 15N14A VL
QAVLTQPSSLSASPGASASLTCTLRSGINVGTYRIYWYQQKPGSPPQYLLSYKSDSDKQQGSGVPSRFSGSKDASANVGILLISGLQSEDEADYYCMIWHSSAWVFGGGTKLTVL

SEQ ID NO: 11 - protein, 118 aa, synthetic construct
  source 1..118: note = 08F17A VH
EVQLVESGGGLVTPGGSLRLSCAASGFTFSGYSMNWVRQAPGKGLEWVSSISSSSGYIYYADSVKGRFTISRDNAKNSLYLQMNSLRAEDTAVYYCAREYWGSGFDYWGQGTLVTVSS

SEQ ID NO: 12 - protein, 107 aa, synthetic construct
  source 1..107: note = 08F17A VL
DIQMTQSPSSLSASVGDRVTITCRASQGISYYLAWYQQKPGKVPKLLISAEFTLQSGVPSRFSGSGSGTDFTLTISSLQPEDVATYYCQKYNTAPRTFGQGTKVEIK

SEQ ID NO: 13 - protein, 7 aa, synthetic construct
  source 1..7: note = 05H21A HCDR1 (Kabat)
TSGMWVS

SEQ ID NO: 14 - protein, 16 aa, synthetic construct
  source 1..16: note = 05H21A HCDR2 (Kabat)
LIDWGDDKYYTTSLKT

SEQ ID NO: 15 - protein, 10 aa, synthetic construct
  source 1..10: note = 05H21A HCDR3 (Kabat)
MREGSRAFDI

SEQ ID NO: 16 - protein, 13 aa, synthetic construct
  source 1..13: note = 05H21A LCDR1 (Kabat)
SGSSSNIGDNAVN

SEQ ID NO: 17 - protein, 7 aa, synthetic construct
  source 1..7: note = 05H21A LCDR2 (Kabat)
NDDLLSS

SEQ ID NO: 18 - protein, 11 aa, synthetic construct
  source 1..11: note = 05H21A LCDR3 (Kabat)
AAWDDSLNGPV

SEQ ID NO: 19 - protein, 5 aa, synthetic construct
  source 1..5: note = 15N14A HCDR1 (Kabat)
SYYWT

SEQ ID NO: 20 - protein, 16 aa, synthetic construct
  source 1..16: note = 15N14A HCDR2 (Kabat)
RIDSSGSSKYNPTLKS

SEQ ID NO: 21 - protein, 11 aa, synthetic construct
  source 1..11: note = 15N14A HCDR3 (Kabat)
GDSGYDWAFDY

SEQ ID NO: 22 - protein, 14 aa, synthetic construct
  source 1..14: note = 15N14A LCDR1 (Kabat)
TLRSGINVGTYRIY

SEQ ID NO: 23 - protein, 11 aa, synthetic construct
  source 1..11: note = 15N14A LCDR2 (Kabat)
YKSDSDKQQGS

SEQ ID NO: 24 - protein, 9 aa, synthetic construct
  source 1..9: note = 15N14A LCDR3 (Kabat)
MIWHSSAWV

SEQ ID NO: 25 - protein, 5 aa, synthetic construct
  source 1..5: note = 08F17A HCDR1 (Kabat)
GYSMN

SEQ ID NO: 26 - protein, 17 aa, synthetic construct
  source 1..17: note = 08F17A HCDR2 (Kabat)
SISSSSGYIYYADSVKG

SEQ ID NO: 27 - protein, 9 aa, synthetic construct
  source 1..9: note = 08F17A HCDR3 (Kabat)
EYWGSGFDY

SEQ ID NO: 28 - protein, 11 aa, synthetic construct
  source 1..11: note = 08F17A LCDR1 (Kabat)
RASQGISYYLA

SEQ ID NO: 29 - protein, 7 aa, synthetic construct
  source 1..7: note = 08F17A LCDR2 (Kabat)
AEFTLQS

SEQ ID NO: 30 - protein, 9 aa, synthetic construct
  source 1..9: note = 08F17A LCDR3 (Kabat)
QKYNTAPRT

SEQ ID NO: 31 - protein, 9 aa, synthetic construct
  source 1..9: note = 05H21A HCDR1 (Chothia)
GFSLSTSGM

SEQ ID NO: 32 - protein, 5 aa, synthetic construct
  source 1..5: note = 05H21A HCDR2 (Chothia)
DWGDD

SEQ ID NO: 33 - protein, 10 aa, synthetic construct
  source 1..10: note = 05H21A HCDR3 (Chothia)
MREGSRAFDI

SEQ ID NO: 34 - protein, 13 aa, synthetic construct
  source 1..13: note = 05H21A LCDR1 (Chothia)
SGSSSNIGDNAVN

SEQ ID NO: 35 - protein, 7 aa, synthetic construct
  source 1..7: note = 05H21A LCDR2 (Chothia)
NDDLLSS

SEQ ID NO: 36 - protein, 11 aa, synthetic construct
  source 1..11: note = 05H21A LCDR3 (Chothia)
AAWDDSLNGPV

SEQ ID NO: 37 - protein, 7 aa, synthetic construct
  source 1..7: note = 15N14A HCDR1 (Chothia)
GGSISSY

SEQ ID NO: 38 - protein, 5 aa, synthetic construct
  source 1..5: note = 15N14A HCDR2 (Chothia)
DSSGS

SEQ ID NO: 39 - protein, 11 aa, synthetic construct
  source 1..11: note = 15N14A HCDR3 (Chothia)
GDSGYDWAFDY

SEQ ID NO: 40 - protein, 14 aa, synthetic construct
  source 1..14: note = 15N14A LCDR1 (Chothia)
TLRSGINVGTYRIY

SEQ ID NO: 41 - protein, 11 aa, synthetic construct
  source 1..11: note = 15N14A LCDR2 (Chothia)
YKSDSDKQQGS

SEQ ID NO: 42 - protein, 9 aa, synthetic construct
  source 1..9: note = 15N14A LCDR3 (Chothia)
MIWHSSAWV

SEQ ID NO: 43 - protein, 7 aa, synthetic construct
  source 1..7: note = 08F17A HCDR1 (Chothia)
GFTFSGY

SEQ ID NO: 44 - protein, 6 aa, synthetic construct
  source 1..6: note = 08F17A HCDR2 (Chothia)
SSSSGY

SEQ ID NO: 45 - protein, 9 aa, synthetic construct
  source 1..9: note = 08F17A HCDR3 (Chothia)
EYWGSGFDY

SEQ ID NO: 46 - protein, 11 aa, synthetic construct
  source 1..11: note = 08F17A LCDR1 (Chothia)
RASQGISYYLA

SEQ ID NO: 47 - protein, 7 aa, synthetic construct
  source 1..7: note = 08F17A LCDR2 (Chothia)
AEFTLQS

SEQ ID NO: 48 - protein, 9 aa, synthetic construct
  source 1..9: note = 08F17A LCDR3 (Chothia)
QKYNTAPRT

SEQ ID NO: 49 - protein, 12 aa, synthetic construct
  source 1..12: note = 05H21A HCDR1 (AbM)
GFSLSTSGMWVS

SEQ ID NO: 50 - protein, 9 aa, synthetic construct
  source 1..9: note = 05H21A HCDR2 (AbM)
LIDWGDDKY

SEQ ID NO: 51 - protein, 10 aa, synthetic construct
  source 1..10: note = 05H21A HCDR3 (AbM)
MREGSRAFDI

SEQ ID NO: 52 - protein, 13 aa, synthetic construct
  source 1..13: note = 05H21A LCDR1 (AbM)
SGSSSNIGDNAVN

SEQ ID NO: 53 - protein, 7 aa, synthetic construct
  source 1..7: note = 05H21A LCDR2 (AbM)
NDDLLSS

SEQ ID NO: 54 - protein, 11 aa, synthetic construct
  source 1..11: note = 05H21A LCDR3 (AbM)
AAWDDSLNGPV

SEQ ID NO: 55 - protein, 10 aa, synthetic construct
  source 1..10: note = 15N14A HCDR1 (AbM)
GGSISSYYWT

SEQ ID NO: 56 - protein, 9 aa, synthetic construct
  source 1..9: note = 15N14A HCDR2 (AbM)
RIDSSGSSK

SEQ ID NO: 57 - protein, 11 aa, synthetic construct
  source 1..11: note = 15N14A HCDR3 (AbM)
GDSGYDWAFDY

SEQ ID NO: 58 - protein, 14 aa, synthetic construct
  source 1..14: note = 15N14A LCDR1 (AbM)
TLRSGINVGTYRIY

SEQ ID NO: 59 - protein, 11 aa, synthetic construct
  source 1..11: note = 15N14A LCDR2 (AbM)
YKSDSDKQQGS

SEQ ID NO: 60 - protein, 9 aa, synthetic construct
  source 1..9: note = 15N14A LCDR3 (AbM)
MIWHSSAWV

SEQ ID NO: 61 - protein, 10 aa, synthetic construct
  source 1..10: note = 08F17A HCDR1 (AbM)
GFTFSGYSMN

SEQ ID NO: 62 - protein, 10 aa, synthetic construct
  source 1..10: note = 08F17A HCDR2 (AbM)
SISSSSGYIY

SEQ ID NO: 63 - protein, 9 aa, synthetic construct
  source 1..9: note = 08F17A HCDR3 (AbM)
EYWGSGFDY

SEQ ID NO: 64 - protein, 11 aa, synthetic construct
  source 1..11: note = 08F17A LCDR1 (AbM)
RASQGISYYLA

SEQ ID NO: 65 - protein, 7 aa, synthetic construct
  source 1..7: note = 08F17A LCDR2 (AbM)
AEFTLQS

SEQ ID NO: 66 - protein, 9 aa, synthetic construct
  source 1..9: note = 08F17A LCDR3 (AbM)
QKYNTAPRT

SEQ ID NO: 67 - protein, 8 aa, synthetic construct
  source 1..8: note = 05H21A HCDR1 (Contact)
STSGMWVS

SEQ ID NO: 68 - protein, 12 aa, synthetic construct
  source 1..12: note = 05H21A HCDR2 (Contact)
WLALIDWGDDKY

SEQ ID NO: 69 - protein, 11 aa, synthetic construct
  source 1..11: note = 05H21A HCDR3 (Contact)
ARMREGSRAFD

SEQ ID NO: 70 - protein, 9 aa, synthetic construct
  source 1..9: note = 05H21A LCDR1 (Contact)
IGDNAVNWY

SEQ ID NO: 71 - protein, 10 aa, synthetic construct
  source 1..10: note = 05H21A LCDR2 (Contact)
LLIYNDDLLS

SEQ ID NO: 72 - protein, 10 aa, synthetic construct
  source 1..10: note = 05H21A LCDR3 (Contact)
AAWDDSLNGP

SEQ ID NO: 73 - protein, 6 aa, synthetic construct
  source 1..6: note = 15N14A HCDR1 (Contact)
SSYYWT

SEQ ID NO: 74 - protein, 12 aa, synthetic construct
  source 1..12: note = 15N14A HCDR2 (Contact)
WIGRIDSSGSSK

SEQ ID NO: 75 - protein, 12 aa, synthetic construct
  source 1..12: note = 15N14A HCDR3 (Contact)
ARGDSGYDWAFD

SEQ ID NO: 76 - protein, 10 aa, synthetic construct
  source 1..10: note = 15N14A LCDR1 (Contact)
NVGTYRIYWY

SEQ ID NO: 77 - protein, 14 aa, synthetic construct
  source 1..14: note = 15N14A LCDR2 (Contact)
YLLSYKSDSDKQQG

SEQ ID NO: 78 - protein, 8 aa, synthetic construct
  source 1..8: note = 15N14A LCDR3 (Contact)
MIWHSSAW

SEQ ID NO: 79 - protein, 6 aa, synthetic construct
  source 1..6: note = 08F17A HCDR1 (Contact)
SGYSMN

SEQ ID NO: 80 - protein, 13 aa, synthetic construct
  source 1..13: note = 08F17A HCDR2 (Contact)
WVSSISSSSGYIY

SEQ ID NO: 81 - protein, 10 aa, synthetic construct
  source 1..10: note = 08F17A HCDR3 (Contact)
AREYWGSGFD

SEQ ID NO: 82 - protein, 7 aa, synthetic construct
  source 1..7: note = 08F17A LCDR1 (Contact)
SYYLAWY

SEQ ID NO: 83 - protein, 10 aa, synthetic construct
  source 1..10: note = 08F17A LCDR2 (Contact)
LLISAEFTLQ

SEQ ID NO: 84 - protein, 8 aa, synthetic construct
  source 1..8: note = 08F17A LCDR3 (Contact)
QKYNTAPR

SEQ ID NO: 85 - protein, 10 aa, synthetic construct
  source 1..10: note = 05H21A HCDR1 (IMGT)
GFSLSTSGMW

SEQ ID NO: 86 - protein, 7 aa, synthetic construct
  source 1..7: note = 05H21A HCDR2 (IMGT)
IDWGDDK

SEQ ID NO: 87 - protein, 12 aa, synthetic construct
  source 1..12: note = 05H21A HCDR3 (IMGT)
ARMREGSRAFDI

SEQ ID NO: 88 - protein, 8 aa, synthetic construct
  source 1..8: note = 05H21A LCDR1 (IMGT)
SSNIGDNA

SEQ ID NO: 89 - skipped

SEQ ID NO: 90 - protein, 11 aa, synthetic construct
  source 1..11: note = 05H21A LCDR3 (IMGT)
AAWDDSLNGPV

SEQ ID NO: 91 - protein, 8 aa, synthetic construct
  source 1..8: note = 15N14A HCDR1 (IMGT)
GGSISSYY

SEQ ID NO: 92 - protein, 7 aa, synthetic construct
  source 1..7: note = 15N14A HCDR2 (IMGT)
IDSSGSS

SEQ ID NO: 93 - protein, 13 aa, synthetic construct
  source 1..13: note = 15N14A HCDR3 (IMGT)
ARGDSGYDWAFDY

SEQ ID NO: 94 - protein, 9 aa, synthetic construct
  source 1..9: note = 15N14A LCDR1 (IMGT)
SGINVGTYR

SEQ ID NO: 95 - protein, 7 aa, synthetic construct
  source 1..7: note = 15N14A LCDR2 (IMGT)
YKSDSDK

SEQ ID NO: 96 - protein, 9 aa, synthetic construct
  source 1..9: note = 15N14A LCDR3 (IMGT)
MIWHSSAWV

SEQ ID NO: 97 - protein, 8 aa, synthetic construct
  source 1..8: note = 08F17A HCDR1 (IMGT)
GFTFSGYS

SEQ ID NO: 98 - protein, 8 aa, synthetic construct
  source 1..8: note = 08F17A HCDR2 (IMGT)
ISSSSGYI

SEQ ID NO: 99 - protein, 11 aa, synthetic construct
  source 1..11: note = 08F17A HCDR3 (IMGT)
AREYWGSGFDY

SEQ ID NO: 100 - protein, 6 aa, synthetic construct
  source 1..6: note = 08F17A LCDR1 (IMGT)
QGISYY

SEQ ID NO: 101 - skipped

SEQ ID NO: 102 - protein, 9 aa, synthetic construct
  source 1..9: note = 08F17A LCDR3 (IMGT)
QKYNTAPRT

SEQ ID NO: 103 - DNA, 648 bp, synthetic construct
  source 1..648: mol_type = other DNA; note = 05H21A LC
cagtctgtgctgacccagcctccatctgtgtctgaggcccctagacagagagtgaccatctcctgctccggctcctcctctaacatcggcgataacgccgtgaactggtatcagcagctgcctggcaaggcccctaaactgctgatctacaacgacgacctgctgtcctctggcgtgtccgacagattctccggctctaagtctggcacctccgccagcctggctatctctggattgcagtctgaggacgaggccgactactactgtgccgcctgggacgattctctgaacggccctgtttttggcggaggcaccaagctgacagtcctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 104 - DNA, 1350 bp, synthetic construct
  source 1..1350: mol_type = other DNA; note = 05H21A HC
caagtgaccctgagagagtctggacccgctctggtcaagcccacacagaccctgacactgacctgcaccttctccggcttctccctgtccacctctggaatgtgggtgtcctggatcagacagcctcctggcaaggcactggaatggctggctctgatcgattggggcgacgacaagtactacaccaccagcctgaaaacccggctgaccatctccaaggacacctccaagaaccaggtggtgctgaccatgaccaacatggaccctgtggacaccgccacctactactgcgccagaatgagagagggatccagagccttcgatatctggggccagggaaccgtggtcaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 105 - DNA, 663 bp, synthetic construct
  source 1..663: mol_type = other DNA; note = 15N14A LC
caggctgttctgacccagcctagctctctgtctgcttctcctggcgcttccgcctctctgacctgcacactgagatccggcatcaacgtgggcacctaccggatctactggtatcagcagaagcctggcagccctcctcagtacctgctgtcctacaagtccgactccgacaagcagcaaggctctggcgtgccctctagattctccggctctaaggacgcctccgccaatgtgggcatcctgctgatctctggcctgcagtctgaggacgaggccgactactactgcatgatctggcactcctccgcctgggttttcggaggcggaacaaagctgacagtcctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 106 - DNA, 1347 bp, synthetic construct
  source 1..1347: mol_type = other DNA; note = 15N14A HC
caggttcagctgcaagagtctggacccggcctggtcaagccttccgagacactgtctctgacctgcaccgtgtctggcggctccatctcctcttactattggacctggatcagacagcctgccggcaaaggcctggaatggatcggcagaatcgactcctccggctcctccaagtacaaccccacactgaagtccagagtgaccatgtccgtggacacctccaagaaccagttctccctgaagctgtcctccgtgaccgctgctgataccgccgtgtactactgtgccagaggcgactctggatacgactgggcctttgactattggggccagggcacactggtcaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 107 - DNA, 642 bp, synthetic construct
  source 1..642: mol_type = other DNA; note = 08F17A LC
gacatccagatgacccagtctccatcctctctgtccgcttctgtgggcgacagagtgaccatcacctgtagagcctctcagggcatctcctactacctggcctggtatcagcagaaacccggcaaggtgcccaagctgctgatctctgctgagttcaccctgcagtctggcgtgccctctagattctccggctctggctctggcaccgactttaccctgacaatctccagcctgcagcctgaggatgtggccacctactactgccagaagtacaacaccgctcctcggacctttggccagggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 108 - DNA, 1344 bp, synthetic construct
  source 1..1344: mol_type = other DNA; note = 08F17A HC
gaggtgcagctggttgaatctggcggaggactggttacccctggcggatctctgagactgtcttgtgccgcctctggcttcaccttctccggctactctatgaactgggtccgacaggctcctggcaaaggcctggaatgggtgtcctccatctcctccagcagcggctacatctactacgccgactccgtgaagggcagattcaccatctccagagacaacgccaagaactccctgtacctgcagatgaacagcctgagagccgaggacaccgccgtgtactactgtgccagagagtattggggctccggcttcgattattggggccaaggaacactggtcaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 109 - protein, 269 aa, Homo sapiens
  source 1..269: note = IL-1beta (Uniprot ID P01584)
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 110 - protein, 153 aa, synthetic construct
  source 1..153: note = the fragment of SEQ ID NO: 109
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS